SEQUENCE LISTING (61 sequences)

SEQ ID NO: 1 - protein, 22 aa, Homo sapiens
RINPNSGGTNYAQKFQGRVTST

SEQ ID NO: 2 - protein, 22 aa, Homo sapiens
WINAGNGNTKYSQKFQGRVTIT

SEQ ID NO: 3 - protein, 22 aa, Homo sapiens
WMNPNSGNTGYAQKFQGRVTMT

SEQ ID NO: 4 - protein, 22 aa, Homo sapiens
WISAYNGNTNYAQKLQGRVTMT

SEQ ID NO: 5 - protein, 22 aa, Homo sapiens
GFDPEDGETIYAQKFQGRVTMT

SEQ ID NO: 6 - protein, 22 aa, Homo sapiens
WITPFNGNTNYAQKFQDRVTIT

SEQ ID NO: 7 - protein, 22 aa, Homo sapiens
IINPSGGSTSYAQKFQGRVTMT

SEQ ID NO: 8 - protein, 22 aa, Homo sapiens
WIVVGSGNTNYAQKFQERVTIT

SEQ ID NO: 9 - protein, 22 aa, Homo sapiens
GIIPIFGTANYAQKFQGRVTIT

SEQ ID NO: 10 - protein, 22 aa, Homo sapiens
LVDPEDGETIYAEKFQGRVTIT

SEQ ID NO: 11 - protein, 21 aa, Homo sapiens
LIYWNDDKRYSPSLKSRLTIT

SEQ ID NO: 12 - protein, 21 aa, Homo sapiens
HIFSNDEKSYSTSLKSRLTIS

SEQ ID NO: 13 - protein, 21 aa, Homo sapiens
LIDWDDDKYYSTSLKTRLTIS

SEQ ID NO: 14 - protein, 22 aa, Homo sapiens
NIKQDGSEKYYVDSVKGRFTIS

SEQ ID NO: 15 - protein, 22 aa, Homo sapiens
GISWNSGSIGYADSVKGRFTIS

SEQ ID NO: 16 - protein, 22 aa, Homo sapiens
YISSSGSTIYYADSVKGRFTIS

SEQ ID NO: 17 - protein, 21 aa, Homo sapiens
AIGTAGDTYYPGSVKGRFTIS

SEQ ID NO: 18 - protein, 24 aa, Homo sapiens
RIKSKTDGGTTDYAAPVKGRFTIS

SEQ ID NO: 19 - protein, 22 aa, Homo sapiens
GVSWNGSRTHYVDSVKRRFIIS

SEQ ID NO: 20 - protein, 22 aa, Homo sapiens
GVSWNGSRTHYADSVKGRFIIS

SEQ ID NO: 21 - protein, 22 aa, Homo sapiens
GINWNGGSTGYADSVKGRFTIS

SEQ ID NO: 22 - protein, 22 aa, Homo sapiens
SISSSSSYIYYADSVKGRFTIS

SEQ ID NO: 23 - protein, 22 aa, Homo sapiens
AISGSGGSTYYADSVKGRFTIS

SEQ ID NO: 24 - protein, 22 aa, Homo sapiens
VISYDGSNKYYADSVKGRFTIS

SEQ ID NO: 25 - protein, 22 aa, Homo sapiens
VISYDGSNKYYADSVKGRFTIS

SEQ ID NO: 26 - protein, 22 aa, Homo sapiens
VIWYDGSNKYYADSVKGRFTIS

SEQ ID NO: 27 - protein, 22 aa, Homo sapiens
GVSWNGSRTHYADSVKGRFIIS

SEQ ID NO: 28 - protein, 20 aa, Homo sapiens
SISGGSTYYADSRKGRFTIS

SEQ ID NO: 29 - protein, 22 aa, Homo sapiens
LISWDGGSTYYADSVKGRFTIS

SEQ ID NO: 30 - protein, 22 aa, Homo sapiens
YISSSSSTIYYADSVKGRFTIS

SEQ ID NO: 31 - protein, 24 aa, Homo sapiens
FIRSKAYGGTTEYTASVKGRFTIS

SEQ ID NO: 32 - protein, 21 aa, Homo sapiens
VIYSGGSTYYADSVKGRFTIS

SEQ ID NO: 33 - protein, 22 aa, Homo sapiens
AISSNGGSTYYANSVKGRFTIS

SEQ ID NO: 34 - protein, 21 aa, Homo sapiens
VIYSGGSTYYADSVKGRFTIS

SEQ ID NO: 35 - protein, 24 aa, Homo sapiens
RTRNKANSYTTEYAASVKGRFTIS

SEQ ID NO: 36 - protein, 24 aa, Homo sapiens
RIRSKANSYATAYAASVKGRFTIS

SEQ ID NO: 37 - protein, 22 aa, Homo sapiens
RINSDGSSTSYADSVKGRFTIS

SEQ ID NO: 38 - protein, 20 aa, Homo sapiens
SISGGSTYYADSRKGRFTIS

SEQ ID NO: 39 - protein, 21 aa, Homo sapiens
EIYHSGSTNYNPSLKSRVTIS

SEQ ID NO: 40 - protein, 21 aa, Homo sapiens
YIYYSGSTYYNPSLKSRVTMS

SEQ ID NO: 41 - protein, 21 aa, Homo sapiens
YIYHSGSTYYNPSLKSRVTIS

SEQ ID NO: 42 - protein, 21 aa, Homo sapiens
YIYYSGSTYYNPSLKSRVTIS

SEQ ID NO: 43 - protein, 21 aa, Homo sapiens
YIYYSGSTYYNPSLKSLVTIS

SEQ ID NO: 44 - protein, 21 aa, Homo sapiens
EINHSGSTNYNPSLKSRVTIS

SEQ ID NO: 45 - protein, 21 aa, Homo sapiens
SIYYSGSTYYNPSLKSRVTIS

SEQ ID NO: 46 - protein, 21 aa, Homo sapiens
YIYYSGSTNYNPSLKSRVTIS

SEQ ID NO: 47 - protein, 21 aa, Homo sapiens
YIYYSGSTNYNPSLKSRVTIS

SEQ ID NO: 48 - protein, 21 aa, Homo sapiens
SIYHSGSTYYNPSLKSRVTIS

SEQ ID NO: 49 - protein, 22 aa, Homo sapiens
IIYPGDSDTRYSPSFQGQVTIS

SEQ ID NO: 50 - protein, 22 aa, Homo sapiens
RIDPSDSYTNYSPSFQGHVTIS

SEQ ID NO: 51 - protein, 23 aa, Homo sapiens
RTYYRSKWYNDYAVSVKSRITIN

SEQ ID NO: 52 - protein, 22 aa, Homo sapiens
WINTNTGNPTYAQGFTGRFVFS

SEQ ID NO: 53 - protein, 22 aa, Homo sapiens
WFNTYTGNPTYAQGFTGRFVFS

SEQ ID NO: 54 - protein, 106 aa, Rattus norvegicus
DIQMTQSPPSLSASLGDKVTITCQASQDINKYIAWYQQKPGKAPRQLIRYTSILVLGTPSRFSGSGSGRDFSFSISNVASEDIASYYCLQYGNLYTFGAGTKLEIK

SEQ ID NO: 55 - protein, 117 aa, Rattus norvegicus
EVKLLESGPGLVQPSQTLSLTCTVSGFPLTTNGVSWVRQPPGKGLEWIAAISSGGSPYYNSALKSRLSINRDTSKSQVFLKMNSLQTEDTAIYFCTREDGWNYFDYWGPGTMVTVSS

SEQ ID NO: 56 - protein, 107 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGNSYPYTFGGGTKLEIK

SEQ ID NO: 57 - protein, 120 aa, Mus musculus
DVQLVESGGGLVQPGGSRKLSCAASGFTFSSFGMHWVRQAPEKGLEWVAYISSGSSTLHYADTVKGRFTISRDNPKNTLFLQMTSLRSEDTGMYYCARWGNYPYYAMDYWGQGTSVTVSS

SEQ ID NO: 58 - protein, 107 aa, Mus musculus
DIVMTQSHKFMSTSVGDRVSITCKASQDVNTAVAWYQQKPGHSPKLLIYSASFRYTGVPDRFTGNRSGTDFTFTISSVQAEDLAVYYCQQHYTTPPTFGQGTKVEIK

SEQ ID NO: 59 - protein, 120 aa, Mus musculus
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 60 - protein, 111 aa, Homo sapiens
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 61 - protein, 117 aa, Homo sapiens
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

(19) United States
(12) Patent Application Publication
(10) Pub. No.: US 2006/0003412 A1
(43) Pub. Date: Jan. 5, 2006

(54) Protein engineering with analogous contact environments

(75) Inventors: Aaron Keith Chamberlain, Pasadena, CA (US); John R. Desjarlais, Pasadena, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Xencor, Inc., Monrovia, CA (US)

(21) Appl. No.: 11/149,943

(22) Filed: Jun. 9, 2005

Related U.S. Application Data
(63) Continuation-in-part of application No. 11/008,647, filed on Dec. 8, 2004.
(60) Provisional application No. 60/528,230, filed on Dec. 8, 2003.
(60) Provisional application No. 60/602,566, filed on Aug. 17, 2004.

(51) Int. Cl. G06F 19/00; G01N 33/48; C07H 21/04; C12P 21/06; C12N 5/06

(52) U.S. Cl. 435/69.1; 702/19; 435/320.1; 435/328; 530/387.3; 536/23.53

(57) ABSTRACT

The invention relates to novel methods for engineering protein sequences using structural and homology information.

This application claims of benefit under 35 U.S.C. §119(e) to U.S. Ser. Nos. 60/528,230, filed Dec. 8, 2003 and 60/602,566, filed Aug. 17, 2004 and is a continuation-in-part of U.S. Ser. No. 11/008,647, filed Dec. 8, 2004, all incorporated by reference.

### FIELD OF THE INVENTION

The invention relates to novel methods for engineering protein sequences using structural and homology information and has utility in the humanization of antibody sequences.

### BACKGROUND OF THE INVENTION

Throughout evolution, the processes of genetic drift and natural selection have lead to the exploration of countless protein sequences, many with related structures and functions. Using well-known methods of bioinformatics, most naturally occurring protein sequences may be aligned relative to homologues that have related sequences and structures. Ultimately, one creates a multiple sequence alignment (MSA) of numerous members of a protein family, using any of a variety of sequence or structure alignment programs known in the art. A great deal of useful information exists in these sets of related proteins and their sequences. Because they have similar structures and functions, an amino acid found at a particular position in one member of a protein family may be a useful substitution at an equivalent position in an alternative member of the family. Modification of the amino acid sequence of a protein is frequently used to create variant proteins with improved properties, including proteins with higher stability, altered specificity, and altered activity. However, such a strategy often fails due to the complex nature of protein structure and evolutionary sequence changes. An amino acid that is favorable in one protein can thus be unfavorable in a related protein. This issue most typically arises because of strong coupling patterns between two or more amino acids that closely interact in the three-dimensional structure of the protein. Hence, there is a need in the art to more optimally utilize information from multiple sequence alignments.

Accordingly, it is an object of the invention to provide methods for analysis and comparison of related proteins to predict the compatibility or feasibility of novel amino acid sequences with a specified protein structural form. It is an object of the invention to provide methods for combining sequence alignment information with structural information in order to evaluate the compatibility of amino acid combinations within a given protein structural form. It is an object of the present invention to further provide sequence and structure-based scoring functions that may be used to evaluate the fitness of substitutions in a template protein. In a preferred embodiment, said scoring functions evaluate one or more substitutions for their structural compatibility with a protein structure template. It is a further object of the invention to predict structural compatibility by combining sequence alignment information with structural information. The invention finds use in various contexts in which prediction of favorable protein sequences is desired, for example protein engineering including antibody engineering, humanization of antibodies, CDR grafting, chimeric protein creation, the transfer of active site or binding sites, protein stability or specificity prediction, protein identification from databases, or various other protein design and bioinformatics projects.

### SUMMARY OF THE INVENTION

Thus, the present invention provides methods for modifying a template protein to generate a second protein, comprising comparing a structural environment of at least one reference position of the template protein and at least one structural environment of the corresponding at least one reference position of at least one related protein. In some aspects, a number of related proteins are used or tested, with from about 5 to about 10 to about 50 to about 100 different related proteins all being preferred. A scoring function is then used to generate a score for the similarity of said structural environment of said at least one related protein to said structural environment of said template protein. At least one modification for said at least one reference position of said template protein to generate said second protein is selected. The scoring function comprises use of a proximity measure. In some aspects, the structural environments may include single positions (e.g. amino acids) or a plurality of positions.

The scoring function may include a number of components, including but not limited to, the use of proximity values of directly contacting amino acids and indirectly contacting amino acids, evaluation of amino acid similarity values, a simultaneous comparison of proximity values and amino acid similarity values, a non-discrete proximity function, a non-binary comparison of environment similarity, a non-binary comparison of amino acid similarities, structural precedence scores, and relative environmental similarity scores.

In an additional aspect, the method utilizes a frequency function wherein the frequency function uses multiple scores from a scoring function.

In a further aspect, the amino acid chosen to be modified may be chosen based on at least two measures selected from the following: structure-weighted frequency, relative environmental similarity, and precedence.

In an additional aspect, modifications may be chosen based on the highest similarity score, or on a score in the highest 10%, 20%, 30%, 40%, 50%, 60% or 70% of the scores.

In a further aspect, the invention provides methods for modifying a template protein to generate a second protein by comparing a structural environment of at least two reference positions of a template protein and at least one structural environment of the at least two corresponding two reference positions of at least one related protein, using a scoring function to generate a score for the similarity of a structural environment of at least one related protein to a structural environment of a template protein, and selecting at least two modifications for at least two reference positions of a template protein to generate a second protein; wherein a scoring function comprises use of a proximity measure.

In a further aspect, the invention provides methods for modifying a template protein to generate a second protein by comparing a structural environment of at least two reference positions of a template protein and at least one structural environment of corresponding reference positions of at least two related proteins, using a scoring function to generate a score for the similarity of the structural environment of at least one related protein to the structural environment of the template protein, selecting one related protein with a similar structural environment to the template protein, and, selecting at least two modifications for at least two reference positions of the template protein to generate a second protein; wherein said scoring function comprises use of a proximity measure.

In an additional aspect, the invention provides methods for modifying a template protein to generate a second protein, by comparing a structural environment of at least two reference positions of a template protein and at least one structural environment of the corresponding reference positions of at least one related protein, using a scoring function to generate a score for the similarity of the structural environment of the template protein to the structural environment of the related protein; selecting a template protein comprising a similar structural environment to the template protein from the related proteins, and, selecting at least two modifications for at least two reference positions of the template protein to generate the second protein, wherein the scoring function comprises use of a proximity measure.

In a further aspect, the invention provides methods for modifying a template protein to generate a second protein, by comparing a structural environment of at least one reference position of the template protein and at least one structural environment of the corresponding at least one reference position of at least one related protein and selecting at least one modification for at least one reference position of the template protein to generate the second protein.

In an additional aspect, the invention provides methods for modifying a template protein to generate a second protein by comparing a structural environment of at least one reference position of the template protein and at least one structural environment of the corresponding at least one reference position of at least one related protein, using a scoring function to generate a score for the similarity of the structural environment of the related protein to the structural environment of the template protein, and selecting at least one modification for the at least one reference position of the template protein to generate the second protein.

In a further aspect, the invention provides of generating a variant protein sequence by inputting a structure comprising at least a first structural environment of a first set of reference amino acid positions of a template protein into a computer, identifying the corresponding second structural environment of a second set of reference amino acid positions of the second protein, using a computational scoring function comprising a proximity measure to generate a score for the similarity of the first and second structural environments, using the score to identify variant amino acid residues to replace at least one amino acid at one of the positions in the first set, and generating at least one variant protein sequence comprising at least one of the variant amino acid residues to generate a variant protein.

In an additional aspect, the invention provides methods as above further comprising providing a sequence of a third related protein and using a scoring function to generate a score for the similarity of a third structural environment of a third set of reference amino acid positions of the third protein to the first structural environment. That is, structural environments of two related proteins are compared to the template protein. The method may further comprise identifying the structural environment that is similar to the first structural environment, wherein a variant protein sequence comprises at least two variant amino acid residues.

In a further aspect, the invention allows the selection of more than one environment per protein, by using multiple runs. The method may use a scoring function to generate a score for the similarity of a third structural environment of a third set of reference amino acid positions of a template protein to a fourth structural environment of a corresponding fourth set of reference amino acid positions of a second protein, and a score is used to identify preferred variant amino acid residues to replace at least one amino acid at one of the positions in a first set and to replace at least one amino acid at one of the positions in a third set.

In an additional aspect, the invention provides methods of designing a humanized antibody variable domain for a target antigen. The method includes providing structural data comprising a reference set of the measure of the distances between at least one amino acid residue and other amino acid residues in a reference antibody variable domain. In some aspects, the structural data comprises the three-dimensional coordinates of said reference variable domain, and/or the reference set comprises a measure of the distances of every residue with every other residue of the reference domain. Similarly, the structural data can comprise a distance-matrix of the variable domain. In some aspects, the reference domain and the donor domain are the same, and the steps are done simultaneously by inputting the three-dimensional coordinates of the donor domain. In additional aspects, the reference domain and one of said acceptor domains are the same, and the steps are done simultaneously by inputting the three-dimensional coordinates of one of the acceptor domains. The method includes providing the amino acid sequence of a donor, non-human antibody variable domain comprising donor CDRs and donor FRs and providing a plurality of amino acid sequences of acceptor human antibody variable domains comprising acceptor CDRs and acceptor FRs. A suitability score is then calculated for each of the plurality of acceptor domains using distance-weighted similarity scores and identifying a best acceptor domain using the suitability scores. The acceptor human antibody CDRs of the best acceptor domain are replaced with the donor CDRs to form a humanized antibody variable domain amino acid sequence. The sequence is then optionally synthesized.

As for all the aspects outlined herein, the sets may independently contain one amino acid position or a plurality, which may comprise amino acids in either linear sequence form or steric relatedness. In addition, one or more of the protein sequences (e.g. the template protein sequence or one or more of the related sequences) is a consensus sequence, a wild-type sequence, or a variant sequence. In a further aspect, the methods of the present invention may be applied to the humanization of antibodies. In one embodiment of antibody humanization, the complementary determining regions (CDRs) of a non-human antibody are combined with the framework regions (FRs) of a human antibody to create a new antibody. This embodiment is often referred to as CDR grafting, in which a non-human antibody, the “donor”, donates its CDRs to a second antibody, the “acceptor”. See, for example, U.S. Pat. Nos. 5,585,089, U.S. Pat. No. 6,180,370, U.S. Pat. No. 5,693,761, and U.S. Pat. No. 5,693,762, all incorporated by reference. In one embodiment, the methods of the present invention may be applied to a “donor” antibody of non-human origin, e.g. a murine antibody, and a set of human antibody sequences, (e.g., potential “acceptors”), in order to select the preferred human antibody to be the “acceptor: antibody.

### BRIEF DESCRIPTION OF THE DRAWINGS

FIG. 1. A portion of a plurality of human heavy-chain germline antibody sequences arranged in a multiple sequence alignment. Numbering is according to the Kabat system (Kabat, et al., 1991, Sequences and Proteins of Immunological Interest, United States Public Health Service, National Institutes of Health, Bethesda, incorporated by reference). Residues 50 to 70 are shown for 57 different sequences.

FIG. 2. A schematic of an embodiment of the present invention. When assessing the potential for various amino acids to fit at a reference position (X), a template sequence and structure are compared to homologous proteins in the same family (A and B). A comparison is performed such that amino acids most structurally proximal to the reference position are generally considered most important. Thus, although homologue B has a more similar sequence overall (4 out of 6 identities with template), homologue A has a more similar sequence near the reference position, suggesting that F is a superior substitution to V at position X.

FIG. 3. Structure-weighted frequencies, or probabilities, for amino acid substitutions in m4D5 for reference positions 50 through 70. The upper matrix was calculated using the method of the present invention. The lower matrix was calculated using an unweighted frequency count of amino acids observed at each position in the alignment. An underscore in the top row indicates that the reference sequence, m4D5, contains a gap in that position of the multiple sequence alignment. For most positions, the probabilities generated using either method are substantially different. For example, at position 63, the method of the present invention predicts that F and L are favorable amino acids, and that V is less favorable. In contrast, the simple, unweighted, counting method predicts that V is the most favorable.

FIG. 4. Illustration of an embodiment of the invention. A patch of residues may be defined. Proteins in the MSA may be screened to identify one that presents a similar patch environment to the patch environment of the original protein. In this example, the environment of protein 5 best matches the environment of the reference protein. Two possible implementations of the results are also shown. First, the patch of the reference protein may be transferred into the environment of protein 5 to create a new protein. Second, the patch of protein 5 may be transferred into the environment of the reference protein.

FIG. 5. Antibody structure and function. Shown is a model of a full length human IgG1 antibody, modeled using a humanized Fab structure from pdb accession code 1CE1 (James et al., 1999, J Mol Biol 289:293-301) and a human IgG1 Fc structure from pdb accession code 1DN2 (DeLano et al., 2000, Science 287:1279-1283). The flexible hinge that links the Fab and Fc regions is not shown. IgG1 is a homodimer of heterodimers, made up of two light chains and two heavy chains. The Ig domains that comprise the antibody are labeled, and include VL and CL for the light chain, and VH, Cgamma1 (Cγ1, Cg1), Cgamma2 (Cγ2, Cg2), and Cgamma3 (Cγ3, Cg3) for the heavy chain. The Fc region is labeled. Binding sites for relevant proteins are labeled, including the antigen binding site in the variable region, and the binding sites for FcγRs, FcRn, C1q, and proteins A and G in the Fc region.

FIG. 6. Premise of one embodiment of the present invention. For example, a donor antibody has environment residues (here, framework residues) shown as filled circles. Two potential acceptor antibodies have some framework residues that are identical to the donor framework residues (filled circles) and other framework residues that differ from the donor residues (open circles). The methods of the present invention use similarity scores that are calculated in a distance-dependent manner. The similarity score is used to select which acceptor is the better of the two. In calculating the similarity score, the methods of the current invention give more influence to residues close to the CDRs than to those farther from the CDRs. That is, the weights of the residues are larger near the CDRs and smaller at less proximal residues. In contrast, typical methods known in the art (% ID) use a uniform weight to all residues. This difference between the two methods results in different acceptor antibodies being selected. The methods of the present invention would select Acceptor #2 with a lower percent identity (or homology) to the donor frameworks, but with a great percent identity (or homology) in the CDR proximal residues.

FIG. 7. Percent identity and percent homology scores for the alignments of human germline heavy chain 1-2 to the other 52 human germline heavy chain sequences. BLAST (National Center for Biotechnology Information, N.I.H.) was used to measure the percent identity and percent homology (percent positives).

FIG. 8. Proximity values for two reference positions (I29 and F68) in the structure of Herceptin® (trastuzumab) (Genentech/Biogenldec) (pdb accession code 1FVC). I29 and F68 are shown as non-spherical surfaces. Calculated proximity values for all positions in the protein are mapped onto the structure by placing spheres on the Ca coordinate of each position. The volume of each sphere is proportional to the calculated proximity value (calculated with σ=5). These proximities are used in calculating a distance-weighted similarity score.

FIG. 9. Sequence weights for each human antibody heavy chain germline sequence at reference positions 50 through 70 (Kabat numbering), according to the template sequence m4D5 (heavy chain). The σ value of 5 was used in the proximity calculation (Eq. 1), the similarity matrix was BLOSUM62 (Eq. 2), and the temperature factor T was 1 (Eq. 3). Note that the sequence with the highest weight is strongly dependent on the reference position. For instance, although the germline sequence vh_{—}1-45 has the most similar environment to m4D5 at position 50, vh_{—}1-f has the most similar environment to m4D5 at position 51. Shading is used to highlight the larger sequence weights.

FIG. 10. Resim scores found using an embodiment of the present invention that determines the suitability of an environment for a patch of residues. In this example, the aligned sequences are a plurality of antibody Fc domains and the representative structure was the Fc domain of human IgG1 (PDB code 1DN2). The patch residues were in positions 266, 267, 268, 269 and 300. The five sequences from the MSA with the best environments for this patch are shown. The best environment comes from sequence, AAL35303, which differs from the template, 1DN2, sequence at positions 298, 296, and 275.

FIG. 11. A comparison of CDR grafting using the methods of the present invention and a traditional method. The table shows patch resim scores, a distance-weighted similarity score, of the present invention for graphing murine heavy chain CDRs onto various human heavy chain germline sequences. Resim scores of the present invention are shown in the first column for the top 28 scoring human sequences. The third and fourth columns show the percent identity of the human germline sequences to the murine sequence, a commonly used measure used to determine the best human sequence to accept the CDR graft. The percent identity is shown calculated in two manners. In column 3, the percent identity was calculated using all the residues in the variable heavy domain, whereas in column 4 the calculation used only the non-CDR residues in the variable heavy domain. The percent identity values suggest that h_vh_{—}1-2 and h_vh_{—}1-3 are the best acceptor sequences for this CDR graft.

FIG. 12. A graph showing that the methods of the present invention provide distinct information from previous methods of determining the appropriate acceptor sequence for a CDR graph. Plotted are the resim scores and the percent identities calculated for 52 variable heavy chain germline sequences. The resim scores of the present invention do not correlate well with percent identities, demonstrating that the methods of the present invention provide novel information useful in determining the best human acceptor sequence to receive the CDR graft.

FIG. 13. A table showing heavy chain germline amino acids found in the positions most proximal to the CDR regions. The donor or template sequence, the murine sequence, for the CDR graft is shown in the top row. Amino acids in the human germline sequences are shown below. The proximities of each position to the CDR graft region, or patch, are shown in decreasing order toward the right. Resim scores for each possible acceptor sequence are shown in the first column, so that the most favorable acceptor sequence is h_vh_{—}3-73.

FIG. 14. Sequences and numbering of the heavy and light chains from the PDB structure 1C5D in each of their multiple sequence alignments used herein. Region used as the patch for the CDR grafting example in EXAMPLE 5 are shown with grey highlights around the position numbers (Xencor CDRs).

FIG. 15. Mutations in the donor antibody, PDB structure 1C5D, required to create the humanized antibodies selected from the methods of the present invention and a typically used method. Residues shown in green must be mutated to create the humanized antibodies chosen using both the methods of the present invention and the method of highest sequence identity. Additionally, residues in purple must be mutated to create the antibody chosen by the methods of the present invention and residues in white must be mutated to create the antibody chosen by the method of highest sequence identity.

FIG. 16. Sequences and numbering of the heavy and light chains from the PDB structure 1IGC in each of their multiple sequence alignments used. Region used as the patch for the CDR graft are shown with grey highlights around the position numbers (Xencor CDRs).

FIG. 17. Mutations in the donor antibody, PDB structure 1IGC, required to create the antibodies suggested from the methods of the present invention and a typically used method. Residues shown in green must be mutated to create the humanized antibodies chosen using both the methods of the present invention and the method of highest sequence identity. Additionally, residues in purple must be mutated to create the antibody chosen by the methods of the present invention and residues in white must be mutated to create the antibody chosen by the method of highest sequence identity.

FIG. 18. Sequences and numbering of the heavy and light chains from m4D5 in each of their multiple sequence alignments used herein. Regions used as the patch in the CDR graft are shown with grey highlights around the position numbers (Xencor CDRs).

FIG. 19. Mutations in the donor antibody, m4D5, required to create the antibodies suggested from the methods of the present invention and a typically used method. The mutations are shown on the structure (PDB 1N8Z) of a humanized version of m4D5 referred to as trastuzamab (Herceptin®, Genentech). Residues shown in green must be mutated to create the humanized antibodies chosen using both the methods of the present invention and the method of highest sequence identity. Additionally, residues in purple must be mutated to create the humanized antibody chosen by the methods of the present invention and residues in white must be mutated to create the antibody chosen by the method of highest sequence identity.

FIG. 20. Sequences and numbering of the heavy and light chains from AC10 in each of their multiple sequence alignments used herein. Regions used as the patch in the CDR graft are shown with grey highlights around the position numbers (Xencor CDRs).

FIG. 21. Mutations in the donor antibody, AC10, required to create the antibodies suggested from the methods of the present invention and a typically used method. The mutations are shown on the PDB structure, 1AD9. Residues shown in green must be mutated to create the humanized antibodies chosen using both the methods of the present invention and the method of highest sequence identity. Additionally, residues in purple must be mutated to create the humanized antibody chosen by the methods of the present invention and residues in white must be mutated to create the antibody chosen by the method of highest sequence identity.

FIG. 22. Selecting CDR graft acceptors with weighted patch positions. Preferred CDR graft acceptors for AC10 heavy chain CDRs were determined with and without a bias for CDR residue 61 to have increased importance. (A) The top ten CDR acceptors chosen with the distance-dependant methods of the present invention with and without the residue 61 bias. (B) The 12 most important, e.g. most proximal, environment positions using the residue 61 bias. (C) The 12 most proximal environment positions using equally weighted, e.g. unweighted, CDR patch positions.

FIG. 23. Graph showing resim scores calculated by using proximity as a discrete (ordinate) or continuous (abscissa) function of distance. The CDRs were donated by the heavy chain from the PDB structure 1A2Y.

FIG. 24. CDR grafting with methods of the present invention. A preferred embodiment of the present invention defines a patch to be the CDR regions, such that the framework regions, the environment, is compared. For example, say human germline (1) has CDR's and framework regions that have scores of 90% and 100% to the donor antibody, whereas human germline (2) has CDR's and framework regions that have scores of 100% and 90% to the donor antibody, respectively. Humanization methods that compare framework regions would select acceptor sequence (1) because its framework region is the most similar to the original donor framework region. These methods result in a final humanized antibody that has a 100% score to the original donor antibody. Humanization methods that compare CDR's would select acceptor sequence (2) and result in a humanized antibody containing a lower score to the original framework region.

FIG. 25. resim scores and percent identities of human germline heavy chains as potential acceptors of a CDR graft from the heavy chain in crystal structure 1A2Y. The human germline heavy chain 2-26, h_vh_{—}2-26, is the best graft acceptor according to predictions. The germline heavy chain 4-59, h_vh_{—}4-59, is the best graft acceptor according to the percent identity. This example used Kabat CDRs.

FIG. 26. Same as FIG. 25 except using Xencor CDRs as opposed to Kabat CDRs.

FIG. 27. A method of the present invention and percent identity choices of the best human germline acceptor sequences for various heavy chain sequences taken from PDB structures. The top ranked sequences from the two methods and their scores are shown. A “yes” in the final column designates that the methods of the present invention predicted as the best acceptor a different germline than a typically used measure, the percent identity. The rank of the top-ranked sequence (as judged by methods of the present invention) in the list ranked by % ID methods is shown in column 4. The rank of the top-ranked sequence (as judged by % ID methods) in the list ranked by methods of the present invention is shown in column 8. % ID scores were calculated without and with the CDRs included in the calculation. These examples used Kabat CDRs.

FIG. 28. Same as FIG. 27 except using an alterative system CDRs (known as “Xencor CDRs) as opposed to Kabat CDRs.

FIG. 29. The methods of the present invention and percent identity selections of the best human germline acceptor sequences from a plurality of acceptor sequences for various heavy chain donor sequences of mouse germline heavy chain sequences. The top ranked sequences from the two methods and their scores are shown. A “Yes” in the final column designates that the methods of the present invention predicted as the best acceptor a different germline than a typically used measure, the percent identity. These examples used Kabat CDRs.

FIG. 30. Same as FIG. 29 except using Xencor CDRs as opposed to Kabat CDRs.

FIG. 31. Resim scores and percent identities of human germline kappa light chains as potential acceptors of a CDR graft from the light chain in crystal structure 1B4J. The human germline kappa light chain 2-28, h_vlk_{—}2-28, is the best graft acceptor according to the methods of the present invention predictions. The germline kappa light chain 4-1, h_vlk_{—}4-1, is the best graft acceptor according to the percent identity. This example used Kabat CDRs.

FIG. 32. Resim scores and percent identities of human germline kappa light chains as potential acceptors of a CDR graft from the light chain in crystal structure 1IGM. The human germline kappa light chain 1-12, h_vlk_{—}1-12, is the best graft acceptor according to methods of the present predictions. The germline kappa light chain 1-33, h_vlk_{—}₁-33, is the best graft acceptor according to the percent identity. This example used Xencor defined CDRs.

FIG. 33. The current method and percent identity selections of the best human germline acceptor sequences for various donor, kappa light chain sequences from PDB structures. The top ranked sequences from methods and their scores are shown in columns 2 and 3. The rank of this chosen sequence in the ranked list based on percent identity is shown in column 4. The top-ranked acceptor according to percent identity is shown in column 5 with the percent identities calculated either without or with the CDR's. The rank, as judged by the methods of the present invention, of the top-ranked acceptor, as judged by percent identity, is shown in column 8. A “Yes” in the final column designates that the methods of the present invention predicted as the best acceptor a different germline than predicted by a typically used measure, the percent identity. These examples used Kabat CDRs.

FIG. 34. Same as FIG. 33 except using Xencor CDRs.

FIG. 35. The method of the present invention and percent identity choices of the best human germline acceptor sequences for CDR donors from various mouse germline kappa light chain sequences. The top ranked sequences from the methods of the present invention and their scores are shown in columns 2 and 3. The rank of this chosen sequence (select with the current methods) in the ranked list based on percent identity is shown in column 4. The top-ranked acceptor according to percent identity is shown in column 5 with the percent identities calculated either without or with the CDR's. The rank, as judged by the present methods, of the top-ranked acceptor, as judged by percent identity, is shown in column 8. A “Yes” in the final column designates that the present methods of the present invention predicted as the best acceptor a different germline than predicted by a typically used measure, the percent identity. These examples used Kabat CDRs.

FIG. 36. Same as FIG. 35 except using Xencor CDRs.

FIG. 37 is a BLOSUM similarity matrix. The BLOSUM62 amino acid similarity matrix was derived from amino acid substitution frequencies found in known protein families. (Henikoff, J. G. Amino acid substitution matrices from protein blocks. Proc. Natl. Acad. Sci. USA 89, 10915-10919 (1992)).

FIG. 38 is a distance matrix of a antibody variable domain heavy chain. The distances between residues 1-21 and 1-21 (Kabat numbering) are shown. The distances in this exemplary variable domain heavy chain were derived from the Protein DataBank crystal structure, 1SBS, using amino acid side chain centroids to represent the position of each side chain. Residue pairs that are closer together are shaded darkly and the distances are measured in angstroms.

### DESCRIPTION OF THE INVENTION

The present invention finds utility in identifying exchangeable or importable portions (including single amino acids) of related proteins based on the use of a scoring function, generally a multiparameteric scoring function such as a distance-weighted similarity score. That is, in many instances, it is desirable to combine parts of two different proteins; for example, in antibody therapeutics, it is frequently desirable to combine the antigen specificity of one antibody (e.g. a murine antibody) with the backbone of a human antibody (“humanization”) to result in an antibody with the desired antigen specificity with low immunogenicity in humans. However, the straight combination (i.e. “cut and paste”) of these regions often results in a loss of specificity or affinity, due to the interactions of the two regions in unpredictable ways. Accordingly, the present invention involves the use of computational screening to score the similarity of the structural environments of the regions to allow the importation of the desired functionality into the best acceptor framework. That is, by evaluating the interaction of the desired donor sequence with a plurality of acceptor sequences, the best acceptor sequence, e.g. the acceptor sequence that minimally disrupts the structure of the donor sequence, can be found.

While a variety of methods are disclosed herein, a general method can be described as follows. Structural data about a reference protein is preferably put into a computer. The reference protein can be either a consensus sequence, a donor sequence, an acceptor sequence, a sequence within a family, etc. The structural data comprises a measure of distances between elements (usually the amino acid residues) in a reference set; the reference set is generally the set of distances between every amino acid residue and every other residue, although as outlined herein, subsets of the whole protein can be used (e.g. the reference set comprises the distances between some of the amino acids of the protein). As outlined below, the distances can be generated as a function of all the atoms of the side chain, the alpha carbons only, etc. A distance-weighted similarity score is then generated for each pair of residues, which takes into account the distance between the residues, with closer residues getting a better weight, and similarity between the residues, e.g. how similar are the two residues. All of the distance-weighted similarity scores are summed for the particular comparison to generate a “suitability” score that is used to rank the particular donor/acceptor pair. The “best” acceptor is then chosen as the one having the best suitability score, although high ranking but not “best” acceptors may also be selected. The donor region is then grafted into the acceptor framework.

As is described herein, the present invention can be used in the design of variant proteins, which contain at least one modification as compared to a pre-existing protein. “Modification” in this sense means the insertion, deletion or substitution of any atoms or collections of atoms, most particularly amino acids. That is, in preferred embodiments, the modification is the insertion, deletion or substitution of amino acids.

In the present invention, a particular position or region of the protein is designated as the “reference position”. In the case of multiple amino acids, for example, this is sometimes referred to a “reference region” or “patch region”. The reference or patch region may contain one or more positions in the protein. In the case of antibodies, the reference region can be the CDRs, as described herein. The remaining positions within the protein are sometimes referred to as the “environmental” or “framework” regions, e.g. the non-CDR regions. One aspect of the present invention is the assessment of the compatibility of a reference region of a template protein and one or more structural environment regions of a second, related protein. That is, by using the scoring function(s) as defined herein, the similarity of the structural environment of a first region (either reference or environmental) of a template protein is compared to the structural environment of the corresponding region in a second related protein. Depending on the desired objective, the reference region may be considered the “variable” region or the “fixed” region in a protein design. Likewise, the environmental positions may be considered either “variable” or “fixed” depending on the application of the present invention.

For example, one application of the present invention is in CDR grafting. In this design procedure, the CDR (complement-determining region) sequences from one antibody, for example a murine antibody, are substituted into another antibody, for example, a human antibody. With this procedure, a novel antibody molecule may be formed that retains the antigen specificity of the murine antibody yet has reduced immunogenicity as compared to the murine antibody. In this example, the murine CDR regions may be considered fixed and can be designated as the patch of residues in the present invention. The algorithms in the present invention are used to determine the human antibody with the best environment in which to place the patch residues. In this case, the human environment residues would be considered variable. An alternative view of the same procedure is that the human antibody will have its CDR sequences replaced with those of the murine antibody. In this case, the CDR sequences may be considered variable and the remaining positions are considered fixed. Viewed in this manner, the patch residues, the CDR residues, are considered variable. In short, the technology of the present invention may be used to judge the compatibility of the patch residues and the remaining environment residues. For a given protein design goal, the patch residues may be considered fixed and the environment residues variable or the patch residues may be considered variable and the environment residues fixed.

Thus, by comparing structural environments of reference position(s) within a template protein with the corresponding reference position(s) of one or more related proteins (sometimes referred to as “acceptor” proteins), usually a plurality of acceptor proteins, suitably similar structural environments are identified by using a scoring function to generate a suitability score. Once a suitable similar environment is identified by a suitable score, putative variable amino acid positions and/or variant residues at those positions are identified to replace corresponding residues in the template protein. One or more variant protein sequences (either as sequences or as physical proteins) can then be generated. These variants thus contain a modified structural environment at the reference position(s), as components of the environment have been modified to conform with the corresponding structural environment of the second (related) protein.

In addition, this process may be done using a template protein and a set of related proteins, or a single related protein. In the case of sets of related proteins, it may not be necessary to utilize additional structural information; for example, utilizing the structural information for the template protein, and using sequence alignment techniques to graft additional sequences onto the structure can be done. Similarly, this process may be done either simultaneously or sequentially on two reference positions or “patches” within the template protein and the related protein(s).

In addition, while the discussion below generally relates to the use of amino acids in the analysis, it should be recognized that other structural environments of a reference point, including but not limited to additional components of a structural environment of a protein such as the PEGylation structures, fatty acid structures, or glycosylation structures, can be used as to define the structural environments of interest.

Accordingly, the present invention provides methods of generating variant protein sequences. By “protein” herein is meant at least two amino acids linked together by a peptide bond.

By “amino acid” and “amino acid residue” as used herein is meant one of the 20 naturally occurring amino acids or any non-natural analogues that may be present at a specific, defined position. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e. “analogs”, such as peptoids (see Simon et al., 1992, Proc Natl Acad Sci USA 89(20):9367, incorporated by reference) particularly when LC peptides are to be administered to a patient. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chain may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

As used herein, protein includes proteins, oligopeptides and peptides, and includes wild-type proteins, variant proteins, and fragments of either. The peptidyl group may comprise naturally occurring amino acids and peptide bonds, or synthetic peptidomimetic structures, i.e. “analogs”, such as peptoids (see Simon et al., PNAS USA 89(20):9367 (1992), incorporated by reference). The amino acids may either be naturally occurring or non-naturally occurring; as will be appreciated by those in the art, any structure for which a set of rotamers is known or can be generated can be used as an amino acid. The side chains may be in either the (R) or the (S) configuration. In a preferred embodiment, the amino acids are in the (S) or L-configuration. The protein may be any protein for which a three dimensional structure is known or can be generated; that is, for which there are three-dimensional coordinates for each atom of the protein. The structure of the protein is not necessary for using the protein in the present invention. Generally structures can be determined using X-ray crystallographic techniques, NMR techniques, de novo modeling, homology modeling, etc. In general, if X-ray structures are used, structures at 2 Angstrom resolution or better are preferred, but not required. The proteins may be from any organism, including prokaryotes and eukaryotes, with enzymes from bacteria, fungi, extremeophiles such as the archebacteria, insects, fish, animals (particularly mammals and particularly human) and birds all possible.

Suitable proteins (including “starting”, “first”, “template”, “reference”, “donor”, or “acceptor” proteins and related protein(s)) include, but are not limited to, industrial, agricultural and pharmaceutical proteins, including ligands, cell surface receptors, antigens, antibodies, cytokines, hormones, and enzymes. Preferred proteins include antibodies and fragments thereof. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database. Suitable protein backbones include, but are not limited to, all of those found in the protein database compiled and serviced by the Protein Databank (PDB). Specifically included within “protein” are fragments and domains of known proteins, including functional domains such as variable heavy and light chains, enzymatic domains, binding domains, etc., and smaller fragments, such as turns, loops, etc. That is, portions of proteins may be used as well.

In some embodiments, the reference, donor and acceptor proteins and/or related proteins are naturally occurring, e.g. wild-type proteins. Alternatively, the protein may be a consensus sequence of a protein family, and the related proteins (e.g. acceptor proteins) are either members of the family or variants thereof. Alternatively, the protein may be a variant protein. In some embodiments, for example in the case of antibodies, the donor sequences may be monoclonal antibodies, particularly murine or human antibodies, and the acceptor sequences are human germline sequences.

The discussion below centers around the use of the invention for creating human and humanized antibodies, but the invention relates to the use of the technology to any number of proteins, as is described in U.S. Ser. No. 11/008,647, filed Dec. 8, 2004, incorporated by reference in its entirety, particularly the claims as filed.

Accordingly, the present invention provides methods of generating humanized antibody variable domain(s) to a target antigen. By “target antigen” as used herein is meant the molecule that is bound specifically by the variable region of a given antibody. A target antigen may be a protein, carbohydrate, lipid, or other chemical compound.

By “antibody” herein is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes antibody fragments, as are known in the art, such as Fab, Fab′, F(ab′)₂, Fv, scFv, or other antigen-binding subsequences of antibodies, either produced by the modification of whole antibodies or those synthesized de novo using recombinant DNA technologies. Particularly preferred are full length antibodies that comprise Fc variants as described herein. The term “antibody” comprises monoclonal and polyclonal antibodies. Antibodies can be antagonists, agonists, neutralizing, inhibitory, or stimulatory. The antibodies of the present invention may be nonhuman, chimeric, humanized, or fully human.

By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1 (C_{H}1), Cγ2 (C_{H}2), and Cγ3 (C_{H}3). In some mammals, for example in camels and llamas, IgG antibodies may consist of only two heavy chains, each heavy chain comprising a variable domain attached to the Fc region. By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. Thus, the engineered variable regions of antibodies of the invention can be fused to the additional regions of an antibody, including full length antibodies.

Antibodies of the present invention may be nonhuman, chimeric, humanized, or fully human. As will be appreciated by one skilled in the art, these different types of antibodies reflect the degree of “humanness” or potential level of immunogenicity in a human. For a description of these concepts, see Clark et al., 2000 and references cited therein (Clark, 2000, Immunol Today 21:397-402, incorporated by reference). Chimeric antibodies comprise the variable region of a nonhuman antibody, for example V_{H} and V_{L} domains of mouse or rat origin, operably linked to the constant region of a human antibody (see for example U.S. Pat. No. 4,816,567, incorporated by reference). Said nonhuman variable region may be derived from any organism as described above, preferably mammals and most preferably rodents or primates. In one embodiment, the antibody of the present invention comprises monkey variable domains, for example as described in Newman et al., 1992, Biotechnology 10:1455-1460, U.S. Pat. No. 5,658,570, and U.S. Pat. No. 5,750,105, incorporated by reference. In a preferred embodiment, the variable region is derived from a nonhuman source, but its immunogenicity has been reduced using protein engineering. In a preferred embodiment, the antibodies of the present invention are humanized (Tsurushita & Vasquez, 2004, Humanization of Monoclonal Antibodies, Molecular Biology of B Cells, 533-545, Elsevier Science (USA), incorporated by reference). By “humanized” antibody as used herein is meant an antibody comprising a human framework region (FR) and one or more complementarity determining regions (CDRs) from a non-human (usually mouse or rat) antibody. The non-human antibody providing the CDRs is called the “donor” and the human immunoglobulin providing the framework is called the “acceptor”. Humanization relies principally on the grafting of donor CDRs onto acceptor (human) V_{L} and V_{H} frameworks (Winter U.S. Pat. No. 5,225,539, incorporated by reference). This strategy is referred to as “CDR grafting”. It should be noted, however, that in some cases, both the donor sequences and the acceptor sequences are human; that is, increased functionality may be achieved by grafting human donor sequences into human acceptor sequences.

In one embodiment, the antibody is a variable region. By “variable region” as used herein is meant the region of an immunoglobulin that comprises the N-terminal region of an Ig heavy or light chain and that are responsible for the specificity of the antibody. Particular heavy and light chain variable regions are defined in Igs using the Kabat system. “Kabat et al.” is used herein as a reference to the manuscript, Kabat, et al., 1991, Sequences and Proteins of Immunological Interest, United States Public Health Service, National Institutes of Health, Bethesda. Kabat et al. define a numbering convention for antibody sequences often used herein. Kabat et al. also define the complementary determining regions (CDRs) of antibodies as positions 24-34, 50-56, and 89-97 in the light chain and 31-35B, 50-65, and 95-102 in the heavy chain, using the numbering of Kabat et al. These positions may be referred to as “Kabat CDRs” or “Kabat-defined CDRs” herein.

“Xencor CDRs” or “Xencor-defined CDRs” as used herein refers to positions 27-32, 50-56, and 91-97 in the light chain and 27-35, 52-56, and 95-102 in the heavy chain using the numbering of Kabat et al.

The method of the invention comprise first providing structural data of a reference protein such as an antibody variable domain that contains CDRs and framework regions (FRs). By “structural information” or “structural data” herein is meant three-dimensional information derived from at least one protein structure. In a preferred embodiment, structural information can be atomic coordinates as derived using x-ray crystallographic methods, NMR methods, or the like. In additional embodiments, structural information can be in the form of interatomic distances; inter-side chain distances; Cα-Cα distances; or Cβ-Cβ distances; amino acid centroid distances; proximity values; a contact matrix; a distance matrix; or consensus information from at least two related protein structures or domains can be used. Structural information can also be derived from experimental analyses that measure the influence of one part of the protein on another part. Examples include mutagenesis, analyses of multiple sequence alignments, phage display, chemical reaction with functional groups as well as others. In one embodiment, the structural data is a reference set of the measures of the distances between at least one amino acid residue and other amino acid residues in a reference region. As outlined herein, the reference set can comprise the measures of the distances between every element and every other element, or subsets of the entire set.

In one embodiment, the structural data is a distance matrix. By “distance matrix” herein is meant a two-dimensional matrix of values wherein the values represent the distance information or structural information of one element, represented in the row, with another element, represented in the column. In uses of a “distance matrix” used herein to refer to proteins, the distances may be measured by any means, including those that are used to derive structural information. A preferred embodiment uses distances derived from the three-dimensional structure of the protein or a similar protein. The elements in the rows and columns of the matrix may be atoms, residues, secondary structures, tertiary structures, domains, or any other region in a protein. The protein may be any type of protein, polypeptide or peptide, protein fragment, region of a protein, including natural and unnatural amino acids.

The protein backbone structure that is used may either include the coordinates for both the backbone and the amino acid side chains, or just the backbone, i.e. with the coordinates for the amino acid side chains removed. If the former is done, the side chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

As will be appreciated by those in the art, the methods of the present invention allow computational testing of “site-directed mutagenesis” targets without actually making the mutants, or prior to making the mutants. That is, quick analysis of sequences in which a number of residues are changed can be done to evaluate whether a proposed change is desirable. In addition, this may be done on a known protein, or on a protein optimized as described herein.

As will be appreciated by those in the art, a domain of a larger protein may essentially be treated as a small independent protein; that is, a structural or functional domain of a large protein may have minimal interactions with the remainder of the protein and may essentially be treated as if it were autonomous. In this embodiment, all or part of the residues of the domain may be variable.

It should be noted that even if a position is chosen as a variable position, it is possible that the methods of the invention will optimize the sequence in such a way as to select the wild type residue at the variable position. This generally occurs more frequently for core residues, and less regularly for surface residues. In addition, it is possible to fix residues as non-wild type amino acids as well.

While three-dimensional structures are preferred to be used, structural information may be generated using modeling by various techniques, including but not limited to structure prediction (Oldziej et al. 2005 Proceedings of the National Academy of Science USA, 102(21):7547-52; Kuhlman et al. 2003 Science, 302(5649):1364-8, both incorporated by reference), and homology modeling (see for example, U.S. Ser. No. 10/218,102, incorporated by reference, and references cited therein).

By “structural environment” herein is meant a region of atoms surrounding one or more specified reference positions of a protein. As outlined herein, the structural environment is preferably defined with higher emphasis for atoms that are closer in space to the reference position and lower emphasis for atoms that are farther in space from the reference position (e.g. distance weighting). In a more preferred embodiment, the atoms are components of amino acids. In a preferred embodiment, the structural environment constitutes atoms within about 0 to about 30 Angstroms from the reference position(s), with atoms with 0 to 15 generally being preferred. In some cases, the entire protein may be considered the structural environment of a particular residue or patch.

In some embodiments, a separate reference domain is used; for example from a template protein. A “template” as used herein is simply a structure or sequence that is used as a reference to be compared to another structure or sequence, and can be a reference protein, a donor protein, or any one of a number of acceptor proteins. Acceptor proteins are generally related proteins. A “related” protein as used herein is a protein that is similar to another protein such that both proteins may be present in the same multiple sequence alignment. A “related” residue in a protein is the residue in the protein that occurs at the same position in a multiple sequence alignment as another residue that is used as a reference.

Alternatively, either the donor domain or one of the acceptor domains serves as the source of the structural data.

Once a reference set of the measures of distances between the elements is established, and the donor sequence is mapped onto the reference set, suitability scores for the grafting of patches from the donor to potential acceptor sequences is done.

IN one embodiment, this is done by calculating the distance weighted similarity scores for each pair of elements to be compared, and then generating a suitability score which is the sum of all the individual distance weighted similarity scores.

A “similarity score” as used herein is a score designed to measure the similarity of two amino acids in corresponding positions. These can be measured using different properties of the amino acids. Useful properties for comparison include amino acid identity, size, charge, hydrophobicity, mutation frequencies in protein families, steric compatibility, and others. Preferred similarity matrices include an identity matrix, PAM matrices and BLOSUM matrices (Altschul, S. F. 1991. Journal of Molecular Biology, 219: 555-665; Atlas of Protein Sequence and Structure, Suppl 3, 1978, M. O. Dayhoff, ed. National Biomedical Research Foundation, 1979; Henikoff S and Henikoff J G. (1992) Proc Natl Acad Sci USA. 89(22):10915-9, all incorporated by reference). The comparisons at individual positions in the protein may be combined to generate a final score representing the similarity of the overall sequences, referred to herein as a “global similarity score”.

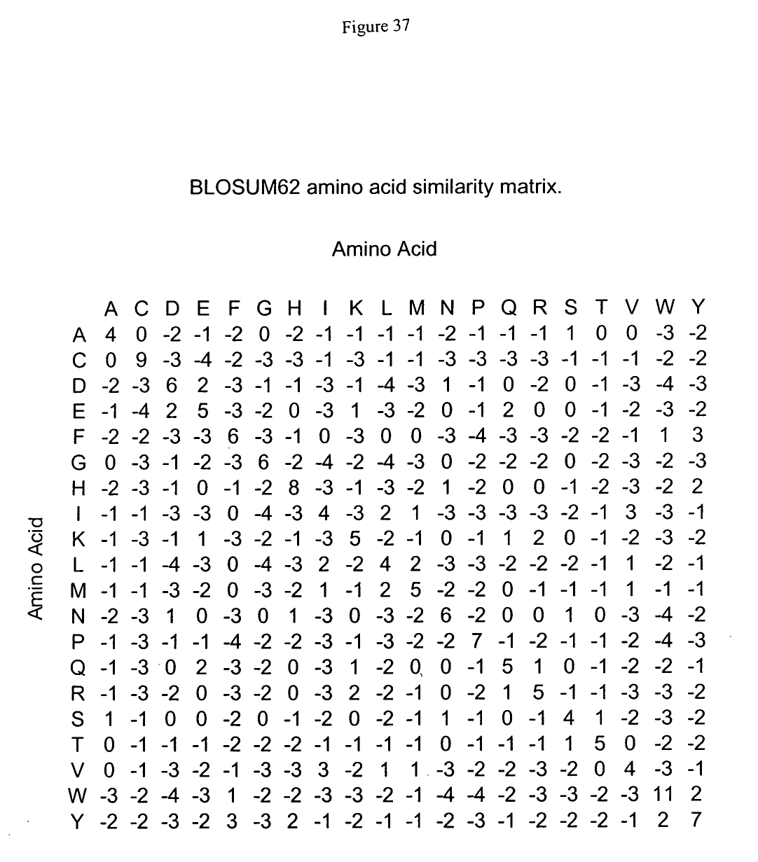

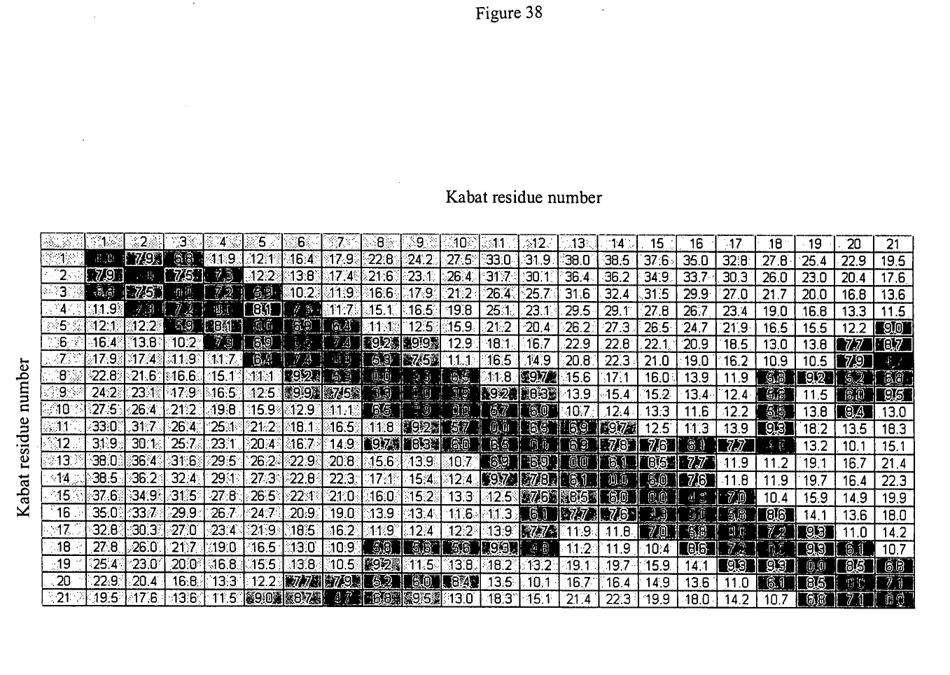

In one embodiment, the similarity score is determined using a similarity matrix. A “similarity matrix” is a matrix of values establishing the degree of similarity of various elements. The elements may be, for example, the 20 commonly found amino acids, all the natural and unnatural amino acids, other molecules such as sugars and fatty acids, or other entities. In the case wherein the similarity matrix is used to compare amino acids, a value in a certain row and column describes the similarity between the amino acid representing that row and the amino acid representing that column. The values in a similarity matrix can be derived from essentially any property of the elements found in the rows and columns. Properties of amino acids used include substitution frequencies in protein families, hydrophobicity, size and charge. Similarity matrices based on amino acid substitution frequencies are particularly preferred in the present invention and include BLOSUM and PAM matrices (Henikoff S and Henikoff H. G. Proc Natl Acad Sci USA. 1992 Nov. 15; 89(22):10915-9; Dayhoff M. R. et al. (1978) Atlas of Protein Sequences and Structure 5:345-352, both incorporated by reference). A BLOSUM matrix is shown in FIG. 37. An identity matrix may also be used; in this case, only if the two residues being compared are identical will there be a similarity score (e.g. if the residues are different, the score is zero, if they are identical, the score is 1). Variations of similarity matrices that are specific for a particular protein family or class, or organisms, e.g. amino acid substitution frequencies in membrane proteins or frequencies in mammals or bacteria, may also be used in the present invention.

The methods of the invention provide for the use of distance-weighted similarity scores. A “distance-weighted similarity score” or “distance-dependent similarity score” is a similarity score that is calculated by allowing positions nearer to a reference position to have more or less influence, more or less weight, on the final score than other positions. In a preferred embodiment, the nearness of a position to a reference position is determined by the distance from the position to the reference position in three-dimensional space, using for example a structure derived by X-ray crystallography, NMR or molecular modeling techniques. In another embodiment, the nearness of two residues may be determined by the proximity measures described herein. Alternatively, the nearness may be determined by eye using a three-dimensional structure. Other methods of determining the nearness of two positions include experimental methods, such as mutagenesis, double mutant cycles, fluorescence, accessibility to chemical modifying agents, accessibility to a molecule in solution, analysis of multiple sequence alignments, analysis of protein families, multiple species comparisons, and effects of perturbations of the protein on the protein stability, folding, resistance to aggregation, or solubility. Another method to judge the nearness of two positions is the energetic coupling of the positions as measured by theoretical, computational or experimental means. In a preferred embodiment the reference position is a point in space, an atom, amino acid or set of amino acids and the relative influence of, or weights at, each position is a function of distance. In preferred embodiments, the weights decrease with increasing distance.

The weights of each position may be determined from the three-dimensional distances, or other measures of nearness, using various methods as shown herein. The weights in the distance-weighted or distance-dependant similarity score may be calculated for example as a continuous function or discrete (non-continuous) function of the distances. Examples of continuous functions include linear functions, non-linear functions, exponential functions, logarithmic functions, Gaussian functions, trigonometric functions, power functions, and various combinations of functions. Examples of discrete functions include binary, trinary functions, step functions, delta functions, inverse trigonometric functions, and combinations of functions comprising a discrete function. One embodiment of the present invention uses constant weights, i.e., the weights are invariant with, or are a constant function of, distance.

As is known in the art, multiple sequence alignments contain a wealth of information about a set of proteins. A “multiple sequence alignment (MSA)” is a collection of linear sequences in which a correspondence is established between the positions in the sequences. Each sequence in the MSA has a linear array of any type of element, with amino acids and nucleic acids being commonly used elements. The correspondence between elements in different sequences is commonly established by their relationship in the MSA. Alternatively, in the case of protein sequences, the correspondence can be established based on the 3-dimensional position of the amino acids in the protein structures, a “structure-based alignment”. MSA can come from a variety of sources, including databases and their generation from computer algorithms. Examples include, BLAST, PSI-BLAST (National Center for Biotechnology Information, National Institute of Health. U.S.A., Altschul, S. F. et al. (1990) J. Mol. Biol. 215:403-410) and CE (Shindyalov and Bourne (1998) Protein Engineering 11(9) 739-747). SCOP (Murzin A. G.et al. (1995). J. Mol. Biol. 247, 536-540), CATH (Orengo, C. A. (1997) Structure. 5(8):1093-1108), PFAM (Bateman, A et al. Nucleic Acids Research (2004) Issue 32:D138-D141), CLUSTALW (Chenna et al., Nucleic Acids Res. 31(13):3497-3500 (2003)), and BLOCKS (Henikoff et al. Nucleic Acids Res. 28:228-230 (2000), all incorporated by reference).

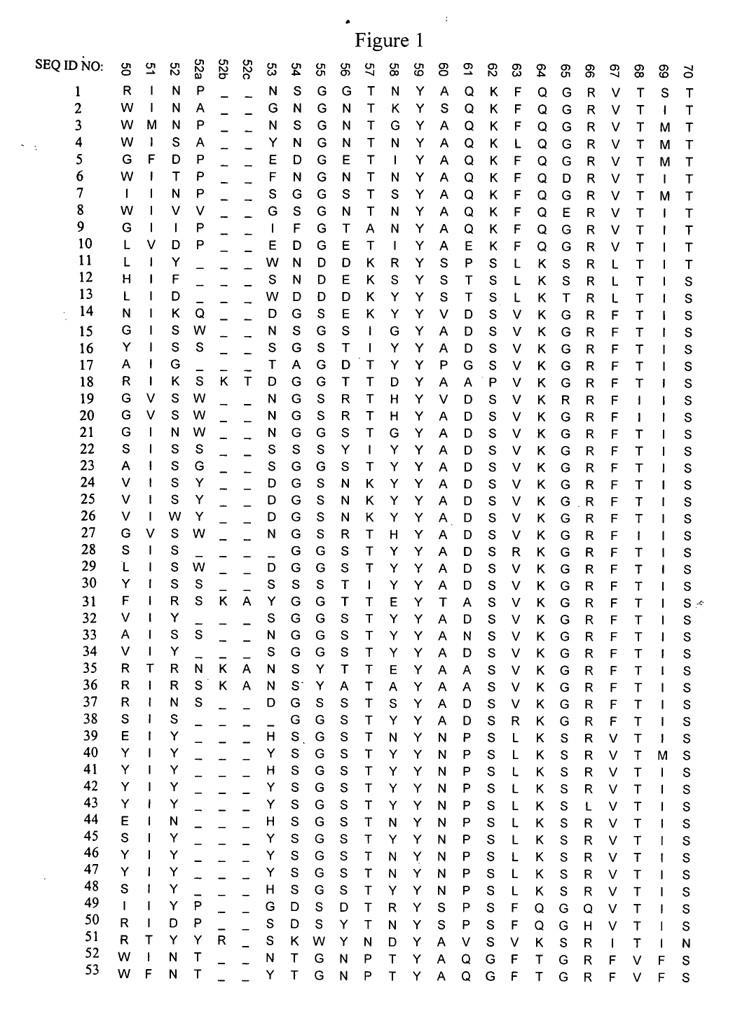

In MSAs, proteins with similar sequences can be aligned to establish which residue in one protein corresponds to another residue in a related protein. Proteins that are similar in sequence often share a common structure or common function and therefore, multiple sequence alignments allow structurally or functionally important residues in a protein to be identified based on knowledge of a related protein. In protein design, the amino acid that could be substituted for another at a particular position in a protein may be decided by using an amino acid found in the corresponding position in a similar protein. If an amino acid has a high frequency at a position in a multiple sequence alignment, that amino acid is said to be “conserved” and the residue is likely to be important for the structure or function of the protein. FIG. 1 shows a multiple sequence alignment of human heavy chain antibody germline sequences. An advantage of the present invention is the combining of information from multiple sequence alignments and protein structures to assess the fitness of an amino acid, or a set of amino acids, for a particular location and environment in a protein.

Another aspect of the present invention is a description of an environment surrounding the amino acid(s) in question (the structural environment), and the use of environment comparisons within related proteins to provide quantitative predictions regarding the compatibility of specific amino acid combinations with the structure in question. The environment comprises many amino acids, each of which contributes to the environment according to its individual properties. In creating the environment, the properties considered by the present invention comprise the similarity of substituting amino acids, the proximity of the environmental residues to the reference position(s) in question, and the overall similarity of the sequences (e.g. a global similarity score).

A typical output of a preferred embodiment is a set of amino acid compatibility or precedence scores for at least one reference position of at least one protein. Extension of this to all reference positions of a protein leads to the definition of a matrix of probabilities and precedence scores denoting the structural compatibility of each amino acid type within each position of a template protein sequence. In an additional embodiment of the present invention, the compatibility of a set of amino acids, a “patch”, and the template protein is assessed. Structural compatibility probabilities for a given position are obtained by taking a weighted frequency count of amino acids observed at equivalent positions in a multiple sequence alignment of related proteins. Structural precedence values are obtained by assessing whether a similar arrangement of amino acids has been observed in an existing protein sequence. The weighting functions are derived by integrating information from the template sequence, each sequence in the MSA (e.g. the set of acceptor proteins), and the three-dimensional structure(s) of one or more members of the protein family.

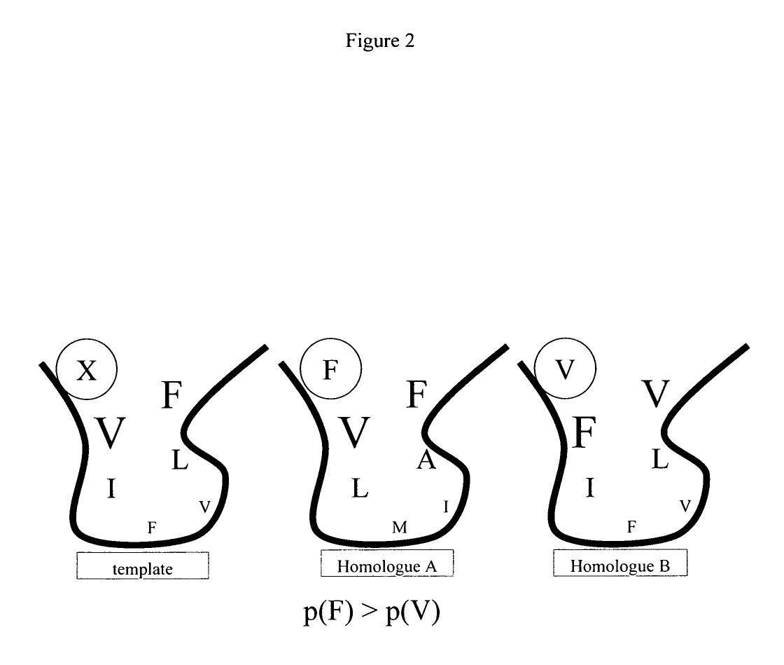

A more typical approach to utilizing MSA information is to take an unweighted frequency count of amino acids observed at equivalent positions in a MSA of related proteins. As is known in the art, this approach may be modified slightly by weighting the contribution of each MSA sequence to the statistics according to its overall dissimilarity to other sequences in the alignment (e.g., as in Henikoff and Henikoff, J Mol. Biol. 1994 Nov. 4;243(4):574-8, incorporated by reference). Unfortunately, this type of analysis is incomplete, leading in many cases to inaccurate predictions. The present invention adds two important features to this type of analysis. First, the similarity of the template sequence to each sequence in the MSA (e.g. the set of acceptor proteins) is considered and contributes to the weighted frequency count. Second, and most importantly, three-dimensional structure information contributes to the weighting procedure: similarities between the template sequence and each MSA sequence are assessed with increased influence for positions that are structurally proximal to the reference position. Thus, if protein A, related to the template protein, has a similar structural environment in the vicinity of reference position X, then the best choice of substitution at position X is the amino found at the corresponding reference position in protein A (FIG. 2).

In one embodiment, the present invention uses the steps of: (a) generating or obtaining a sequence alignment between a template protein and at least one related protein; (b) comparing a template protein and at least one related protein in the structural environment of at least one reference position; (c) evaluating similarity of structural environments between the template protein and at least one related protein (d) using environment similarity scores of each aligned related protein to quantify favorability or compatibility of amino acids at each reference position. It should be emphasized that equivalence or correspondence of reference positions is defined substantially simultaneously for the template protein and each related protein according to the sequence alignment. The structural environment is established using positional proximity measures to the reference position(s). This is generally applied such that the structural environment predominantly constitutes positions close in space to the reference position, while de-emphasizing or excluding positions farther in space from the reference position. Favorability or compatibility information for various amino acids at the reference positions may ultimately be used to select judicious substitutions, predict the stability of various sequences, or to predict interaction affinities (e.g. if the analysis is extended to include multi-subunit proteins or protein-protein and protein-peptide complexes).

In a preferred embodiment, analysis may include the use of a multiple sequence alignment (MSA) comprising the template protein and several related proteins, generating reference position weights for each sequence in the MSA by scoring similarities between the reference position environment of the template protein and corresponding reference position environments of each MSA sequence, and generating probability or structural precedence values for each amino acid at each reference position. In general, more MSA sequences are desirable for the most accurate predictions. However, in some circumstances, small numbers of related proteins may be used to achieve results.

A variety of methods may be applied to evaluate the similarity of two structural environments (one from a template protein and one from a related protein) surrounding equivalent reference positions. The evaluation will generally involve an analysis of the amino acid content of the structural environment and the spatial distribution of amino acids around the reference position (in some embodiments, other chemical entities may be included), and in some cases will further involve analysis of the atomic content and spatial distribution of atoms around the reference position. For example, for cases in which three-dimensional atomistic structures are known or may be constructed or modeled for the sequences in a given MSA, atomistic coordinates may be used to calculate environment similarity. In an alternative embodiment, environment similarity may be calculated by comparing the three-dimensional coordinates of the atoms of the amino acids. The comparison may include the root-mean-squared distance (RMSD) between the coordinates of the amino acid side-chains, the difference in amino acid side-chain dihedral angle values, the amount of overlapping occupied volume shared between the amino acid side-chains, the extent of coordinate overlap of atoms with similar physico-chemical properties (e.g. charge, polarizability, size, and hydrogen-bonding capacity) or the like.

In a preferred embodiment, similarity of structural environment may be evaluated and scored using proximity values—between each environment amino acid position and the reference position—and combined with amino acid similarity comparisons for amino acids in the structural environment.

In a preferred embodiment, proximities may be derived from position-position distances calculated from three-dimensional structures of one or more members of the protein family. Methods for calculating a matrix of side-chain side-chain or position-position distances from a protein structure are well known in the art. These include, but are not limited to, Cα-Cα and Cβ-Cβ distance matrices. In preferred embodiments, centroid-centroid distances are calculated. In an alternative preferred embodiment, the average of all side chain-side chain interatomic distances are calculated to yield a contact distance between each pair of side chains in a protein structure. In another alternative embodiment, distances may be calculated as the point of closest approach of the two side chain atoms. In additional embodiments, one or more distance matrices can be averaged (after appropriate alignment of the matrices to account for gaps/insertions in the different protein structures). In a preferred embodiment, distance values are converted to structural proximity measures or values.

In alternative embodiments, proximities may be derived from visual inspection of a three-dimensional structure, or from mutagenesis or other experimental information, including effects of single, double or higher-order mutations on the protein's stability, structure, folding, function, activity, half-life, affinity for another protein, or other characteristics. Proximities may also be determined from correlated mutations in multiple sequence alignments such as those determined, for example, by Ranganathan et al. (Lockless and Ranganathan, 1999 Science. 286(5438):295-9), incorporated by reference.

In an alternative embodiment, proximities may be calculated by the energetic coupling of two residues in a protein. The energetic coupling may be calculated from experimental mutagenesis, for example, or may be calculated using a three-dimensional structure and energy functions. Appropriate energy functions include those in the Protein Design Automation® programs or PDA® programs (See, e.g., U.S. Pat. Nos. 6,188,965; 6,269,312; 6,403,312; 6,708,120; 6,801,861; 6,804,611 and 6,792,356, and U.S. Ser. Nos. 09/782,004; 09/927,790; 10/101,499; 10/218,102; 10/666,311 and 10/665,307, all incorporated by reference). In a preferred embodiment, the proximity of two residues may be calculated by the effects on the protein energy given perturbations at each residue individually (single perturbations) and a perturbation at both residues simultaneously (double perturbations). Examples of appropriate perturbations include a change in amino acid identity or position at a residue. Energies of the single and double perturbations may be converted into probabilities using the Boltzman equation, as is known in the art. The proximity may be calculated as the mutual information between the two residues, given the singles and doubles perturbation energies.

Proximities may be Used to Calculate a Distance-Dependent Similarity Score.

In a preferred embodiment, structural proximity is calculated with a function that decreases as a function of increasing distance. Examples include, but are not limited to, Gaussian functions (as in Eq. 1), linear functions, decreasing sigmoidal functions, exponential decay functions, and step functions.

$$Equation 1 \text{:}$$

$${proximity}_{ij} = exp ( - 1.0 * {( \frac{{d}_{ij}}{σ} )}^{2} )$$

Where dᵢⱼ is the distance between two residues, i and j. In a preferred embodiment, a Gaussian σ value between 4 and 10 is preferred, with 5 being especially preferred, although other values may be optimal in some situations. This embodiment places highest emphasis on positions directly contacting the reference position, lower emphasis on positions that indirectly contact the reference position, and minimal or no emphasis on positions far in space from the reference position. In the simplest embodiment, proximity is binary—that is, positions have proximities of 1 or 0, as in Equation 1b.

$$Equation 1 b \text{:}$$

$${proximity}_{ij} = \{ \begin{matrix}1 , if {d}_{ij} \leq cutoff distance \\ 0 , otherwise\end{matrix}$$

In some embodiments of Equation 1b, the cutoff distance may be defined such that only amino acids in direct contact with the reference position have nonzero proximities. To achieve this, the distance cutoff should be in the range of about 3-6 angstroms. However, in some embodiments, direct contact is best confirmed or established using visual inspection of the structure.

In a preferred embodiment, amino acid similarity is calculated using an amino acid similarity matrix. Such scoring methods, well known in the art of bio-informatics, may be used to quantify the extent of similarity between two amino acids. Similarity matrices, including but not limited to BLOSUM62, provide a quantitative measure of the compatibility between a sequence and a target structure, which can be used to predict non-disruptive substitution mutations (Topham et al., 1997, Prot. Eng. 10: 7-21). Similarity matrices include, but are not limited to, the BLOSUM matrices (Henikoff & Henikoff, 1992, Proc. Nat. Acad. Sci. USA 89: 10917, incorporated by reference), the PAM matrices, the Dayhoff matrix, and the like. For a review of similarity matrices, see for example Henikoff, 1996, Curr. Opin. Struct. Biol. 6: 353-360, incorporated by reference. Similarity matrices may also be based on specific properties of amino acids such as hydrophobicity, volume, charge, polarity, polarizability, or isostericity. In some embodiments, amino acid similarity can be replaced with the binary comparison of amino acid identity: nonidentical amino acids have scores of 0 and identical amino acids have scores of 1.

In a preferred embodiment, structural proximity values and amino acid similarity measures are combined to determine the structural environment similarity score (esim) between the template protein and each related protein (in the MSA) at a specified reference position. In a preferred embodiment, this environment score is calculated as in Equation 2, where s is the MSA sequence, i is the reference position, aaⱼ^{S} is the amino acid at position j of sequence s, and aaⱼ^{template} is the amino acid at position j of the template sequence (note that position numbers i and j are defined according to the MSA, not according to the numbering of each individual protein), and the score is a sum over all positions in the sequences.

$$Equation 2 \text{:}$$

$$esim ( s , i ) = {\sum }_{j \neq i}^{positions} {proximity}_{ij} \cdot S ( {aa}_{j}^{s} , {aa}_{j}^{template} )$$

where S is a similarity score, e.g. BLOSUM62, for comparing the similarity of two amino acids. Esim scores are examples of a distance-weighted similarity score. Similarities are incorporated by the S matrix and the distance-dependence is incorporated by the proximity(i,j). An optional position-specific weighing function can be added to the above summation to change the influence of different positions in the alignment. For example, the weighing function may be added to change the influence of positions buried in the interior of the protein relative to the surface-exposed residues. The position-specific weighing function may alternatively be used to isolate positions of interest by assigning a zero value to other positions. Positions of interest may include buried positions, exposed positions, positions for which a charged residue is present in the MSA, or positions near a binding site. In a preferred embodiment, the structural proximity of adjoining backbone residues is reduced or set to zero (i.e., j≠i is replaced by |j−i|>w in Equation 2, with w ranging from 1 to 10) in order to emphasize three-dimensional structure information over local backbone structural information.

In an alternative embodiment, the environment similarity score (esim) may be calculated as in Equation 2b, where a binary identity comparison is used, in contrast to the use of a similarity matrix in Equation 2.

$$Equation 2 b \text{:}$$

$$esim ( s , i ) = {\sum }_{j \neq i}^{positions} {proximity}_{ij} \cdot δ ( {aa}_{j}^{s} , {aa}_{j}^{template} )$$

wherein, δ is the Kronicker delta function, defined such that δ(x,y)=1 if x=y and δ(x,y)=0 if x≠y. As will be appreciated, the combination of the simplest form of proximity (Equation 1 b) with Equation 2b will result in simple integer environment similarity scores. That is, an environment score equals the number of residues that are identical to the reference sequence and within a certain proximity of the reference position.

In additional embodiments, the environment similarity of a MSA sequence can be used as a look-up score for identifying the most similar sequence to the given template sequence near the reference position or patch. In this manner, the present invention is useful in a manner similar to BLAST (National Center for Biotechnology Information, National Institute of Health, USA, Altschul, S. F. et al. (1990) J. Mol. Biol. 215:403-410, incorporated by reference) or other algorithms that identify similar proteins to a given protein from a large database. Using the methods of the present invention to select similar proteins to a reference or template protein has utility, for example, in the field of antibodies and antibody humanization. The methods of the present invention may be used during humanization, for example, to select a human antibody from many human antibodies to accept a graft of the complementary determining regions (CDR's) from an original, non-human antibody.

In some embodiments, the environment similarity score can be used directly to generate the final weights of each sequence in the alignment. In preferred embodiment, the final weights are generated by an additional amplification function (such as an exponential), then normalized such that all weights sum to a total probability of 1. In equation 3, an exponential amplification is used with a temperature factor (T) to modulate the extent of amplification, with an optional sequence-dependence weight, h(s), for each sequence in the MSA that is used to further bias the influence of some sequences (e.g. as in Henikoff and Henikoff, J Mol. Biol. 1994 Nov. 4;243(4):574-8), incorporated by reference.

$$Equation 3 \text{:}$$

$$weight ( s , i ) = h ( s ) * \frac{exp ( esim ( s , i ) / T}{{\sum }_{{s}^{′} = 1}^{N} exp ( esim ( {s}^{′} , i ) / T )}$$

The sequence-dependent weighing function, h(s), may be used, for example, to change the influence of sequences with a preferred property. These preferred properties include, for example, favorable binding characteristics to another protein, co-factor, substrate, macromolecule, or other entity, favorable in vivo pharmacodynamic properties, favorable activity characteristics, or favorable expression, solubility, stability, resistance to aggregation or proteases, or structural similarity. Preferred properties need not be limited to physical properties, and may include, for example, properties related to the perception or marketing value of particular sequences.

Once weights for each sequence at each reference position are calculated, amino acid probabilities may be generated for each reference position with a weighted count over amino acids found at this position in the MSA. These probabilities are referred to as the structure-weighted probabilities or structure-weighted frequencies.

$$Equation 4 \text{:}$$

$$f ( aa , i ) = {\sum }_{s = 1}^{N} weight ( s , i ) \cdot δ ( aa , {aa}_{i}^{s} )$$

wherein, δ is the Kronicker delta function, defined such that δ(x,y)=1 if x=y and δ(x,y)=0 if x≠y.

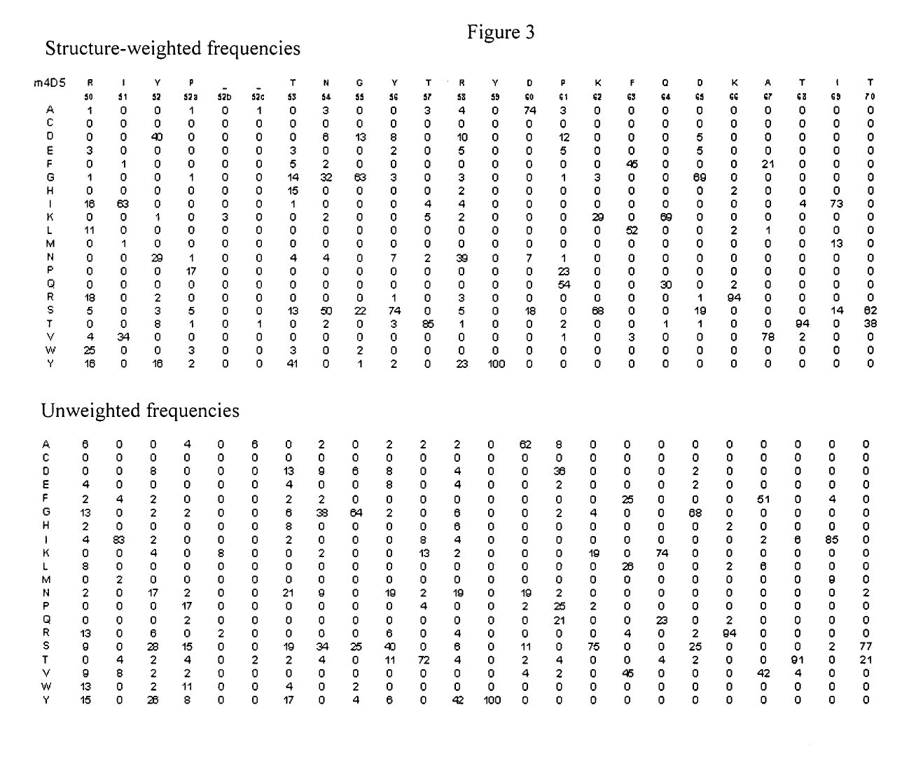

These structure-weighted frequencies differ from the simple amino acid frequencies commonly used in multiple sequence alignments. As in known in the art, the amino acid frequency in each position of the MSA can be used to identify a common amino acid for that position. Because the present invention uses structure-weighted frequencies, the output reflects the compatibility of an amino acid in a particular three-dimensional environment. Structure-weighted frequencies at many positions in the antibody heavy chain are compared to typical frequencies in FIG. 3. In that example, the structure-weighted frequencies are shown in the top panel whereas the unweighted frequencies are in the lower panel. The frequencies are shown as percentages for heavy chain positions 50-70.

In an alternative embodiment, the amino acid probabilities f(aa,i) are converted to log-odds ratio scores using Equation 5.

$$Equation 5 \text{:}$$

$$Lscore ( aa , i ) = log ( \frac{f ( aa , i )}{q ( aa )} )$$

where Lscore(aa,i) is log-odds ratio score of amino acid aa at position i based on environment-based sequence weights. The denominator, q(aa), is the overall frequency of amino acid aa found in general. This frequency may be taken from amino acid frequencies calculated from a variety of sources, include the amino acid frequencies found in all proteins in the MSA, all proteins in a particular organism, or all known proteins in every organism, or all proteins in a protein sequence database (e.g. swissprot).

In an alternative embodiment, the relative environment similarity of a sequence at reference position i is calculated relative to a “perfect” environmental match as determined with the template sequence itself, as follows.
resim(s,i)=exp((esim(s,i)−esim(template,i))/T) Equation 6
where esim(s,i) is determined as in Equation 2.

Thus, a resim(s,i) value of 1.0 implies that, at reference position i, MSA sequence s has a structural environment identical to that of the template sequence. Resim values have the convenience of ranging from 0 to 1, although methods that scale these values to other ranges may be used, as may other methods that also scale values to range from 0 to 1. This alternative scoring system is useful for determining structural precedence for each possible amino acid at each position within the template sequence. In a preferred embodiment, the structural precedence for each amino acid at position i is quantified using the highest resim(s,i) value for all aligned sequences (MSA) that possess that amino acid at position 1, as in equation 7.

$$Equation 7 \text{:}$$

$$precedence ( aa , i ) = {Max}_{s ∈ MSA} ( resim ( s , i ) \cdot δ ( aa , {aa}_{i}^{s} ) )$$

Alternatively, precedence for each amino acid at position i is scored using amino acid similarity instead of identity, as in Equation 7b.

$$Equation 7 b \text{:}$$

$$precedence ( aa , i ) = {Max}_{s ∈ MSA} ( resim ( s , i ) \cdot S ( aa , {aa}_{i}^{s} ) )$$

Alternative precedence could also be calculated using the weight(s,i) as calculated in equation 3:

$$Equation 7 c \text{:}$$

$$precedence ( aa , i ) = {Max}_{s ∈ MSA} ( weight ( s , i ) \cdot δ ( aa , {aa}_{i}^{s} ) )$$

As will be appreciated by those in the art, a variety of possibilities exist for scoring precedence, defined to quantify the extent to which a particular amino acid has already been observed in a protein in the context of a particular structural environment. The functional forms of Equations 2 and 6 also have the advantage that positions with a small number of proximal neighbors (i.e. positions at or near the surface of the protein) will generally have higher precedence values, consistent with expectations of the art.

In additional embodiments, Bayesian statistics methods can be used to further enhance the analysis, particularly when the MSA contains a small number of sequences (e.g., as in Sjolander et al., 1996, Comput. Appl. Biosci. 12(4): 327-345), incorporated by reference. The use of Bayesian statistics allows the introduction of prior information to augment the analysis of amino acid frequencies (as calculated in equation 4). In preferred embodiments, prior information is incorporated using Dirichlet mixtures. In alternate embodiments, prior information may be incorporated using pseudo-counts, similarity matrix mixtures, or a common ancestor analysis.

In additional embodiments, structure-weighted frequencies f(aa,i) and precedence(aa,i) or Lscore(aa,i),may be averaged or summed over the whole sequence to generate a composite score that incorporates information from all positions. Methods of averaging, include, but are not limited to, geometric mean, algebraic mean, sum of squares, and other like methods.

In a preferred embodiment, information from structure-weighted frequencies, precedence scores, environment similarity scores, and averages thereof are utilized to predict or select novel protein sequences with favorable properties. That is, the information may be used to select appropriate modifications to the template protein or any of the related proteins with which it is compared. In a preferred embodiment, amino acids with scores above a user-specified threshold value are selected for substitution into the reference position. This can be done for one or more reference positions. In alternative embodiment, amino acids with the highest scores are selected for substitution into the reference position(s). In additional embodiments, modifications with scores that rank within a given percentile of scores may be used to guide the selection of modifications. In a preferred embodiment, modifications with scores that rank within the top 10% of scores are selected for substitution. In alternative embodiments, modifications with scores that rank within the top 50% of scores are selected for substitution. It will also be appreciated by those skilled artisans that in some cases, where other constraints apply, the user may use output from a method of the present invention to make a more subjective determination of the most appropriate amino acid substitutions. In less preferred but possible embodiments, modifications with particularly low scores may be selected (e.g., if testing hypotheses, or if dramatic perturbation of structural properties is desired).

Consensus Sequence Generation

One embodiment of the present invention is the calculation of a consensus sequence to represent the family of proteins in a MSA. Consensus design is based on the use of a single consensus sequence to represent a MSA. A generic approach to constructing a consensus sequence is to take the most frequently observed amino acid at equivalent positions in the MSA. This is equivalent to constructing the sequence with the maximum probability of being generated using the observed amino acid distributions at each position. That is, of all possible sequences, the consensus sequence is the one that maximizes the quantity, Z, shown in equation 8.

$$Equation 8 \text{:}$$

$$Z = {\sum }_{i}^{positions} log {f}_{MSA} ( aa , i )$$

where aa is a consensus amino acid and ƒ_{MSA}(aa,i) is the frequency of observing aa at position i in the MSA. However, consensus sequences constructed in this manner may contain amino acids in foreign contacting environments since the process of collecting the most frequently observed amino acid at a position does not consider other positions. The present invention adds two important features to this type of analysis. First, the similarity of the consensus sequence to each sequence in the MSA is considered and contributes to the weighted frequency count. Second, and most importantly, three-dimensional structure information contributes to the weighting procedure: similarities between the consensus sequence and each MSA sequence are included with increased weight for positions that are structurally proximal to each reference position. A consensus sequence using the methods of the present invention may be constructed by finding the amino acid sequence with the maximum environment-weighted probability, Z, as described in equation 9.

$$Equation 9 \text{:}$$

$$Z = {\sum }_{i}^{positions} log f ( aa , i \vert consensus )$$

where ƒ(aa,i|consensus) is determined using Equation 4 as discussed earlier. These environment-weighted probabilities depend on the context of the surrounding residues, which necessitates an iterative procedure for determining the consensus sequence of the present methodologies. In a preferred embodiment, a simulated annealing procedure is used to solve for the consensus amino acid sequence that maximizes the quantity found in equation 9. In alternative embodiments, genetic algorithms or Tabu searches may be used to solve for the consensus amino acid sequence. See, for example, U.S. Ser. Nos. 10/218,102, 09/877,695, and 10/071,659, all incorporated by reference. In alternative embodiments, steepest-descent or conjugate-gradient minimization techniques can be used from a consensus starting point to generate a modified or corrected consensus.

Patch Mode

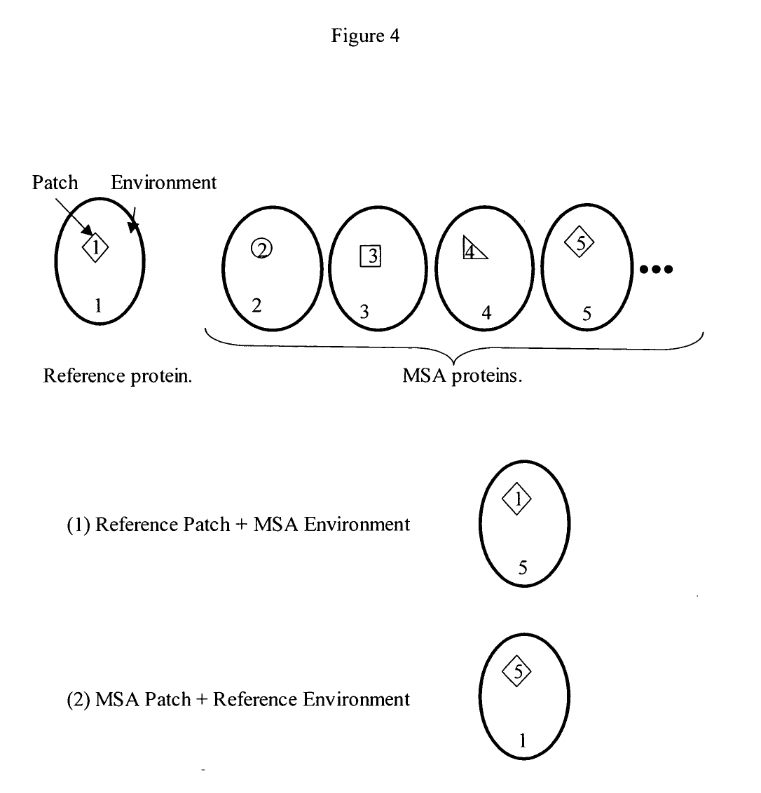

Another embodiment of the present invention is the assessment of the compatibility of a group of amino acids, or patch of amino acids, with given structural environment (FIG. 4). This manner of implementing the present invention may be referred to as “Patch Mode”. In a preferred embodiment, the resim score is used to judge the fitness of the patch from one protein and the environment of related proteins, although the structure-weighted frequency and precedence scores may also be used. In this embodiment, the user specifies the patch, a set of positions in the parent protein, and the algorithm calculates a resim score for each sequence in the MSA. The patch may be any number of positions from 1 to the total number of positions in the multiple sequence alignment minus 1 position, with about 2 to about 30 residues being commonly used. The resim score reflects the extent of similarity of the environment around the patch in the reference or template protein structure to the environment found in each related protein in the alignment. It should be emphasized that it is not necessary for the patch residues to be continuous in sequence or nearby in the three-dimensional structure, although the latter is a commonly used.

Information from the patch analysis may be used to selected suitable replacement patches from related proteins that have similar structural environments to the parent protein, and the template protein may be modified accordingly to generate a second protein. Alternatively, once a related protein with a similar patch structural environment is discovered, it may be selected as a host in which to graft the template protein patch. The choice of direction depends on the intended effect of the modification. For example, the patch of residues may be selected to be the complementary determining regions, CDR's, of an antibody. In this case, the environment comprises the framework regions (FR's). The methods of the present invention may be used to select the antibody that is the best scoring of all antibodies in a plurality. A new antibody may be created in at least two ways, depending on the desired effect. The new antibody may have the CDR's of the template antibody and the framework regions of the selected antibody, or alternatively, the new antibody may have the CDR's of the selected antibody and the framework regions of the template antibody.

The use of many residues in the patch requires only slight adjustments to the equations described above wherein the “patch” was only one position, the reference position i. With multiple residues in the patch, the environmental similarity score is calculated similarly (equation 10).

$$Equation 10 \text{:}$$

$$esim ( s , P ) = {\sum }_{j ∉ P}^{positions} {proximity}_{Pj} \cdot S ( {aa}_{j}^{s} , {aa}_{j}^{template} )$$

“s” still refers to the MSA sequence in question, and “S” still refers to the similarity of each non-patch amino acid at position “j” in the template and sequence “s”. In Patch mode, however, the “P” now refers to a set of patch residues. The summation is done over all positions, j, that are not included in the patch set of residues.

Previously, the proximity(i,j) value was the proximity of residue j and the reference amino acid, i. In patch mode, the proximity(P,j) is commonly taken as the largest proximity value found between residue j and any residue in the patch. That is:

$$Equation 11 \text{:}$$

$${proximity}_{Pj} = {{max}_{k ∈ P}}^{( {proximity}_{k , j} )}$$

Other methods of determining the proximity of the patch and an environment residue are also suitable. For example, the average proximity, or minimum proximity, of the patch residues to the environment residues may be used as the sum of the proximities from each patch residue to the environment residue. A position-specific weighing function can be incorporated into Eq. 11 to allow different patch residues to have more or less influence as in Equation 11b.

$$Equation 11 b \text{:}$$

$${proximity}_{Pj} = {{max}_{k ∈ P}}^{( w ( k ) * {proximity}_{k , j} )}$$

where w(k) is the weights of each patch residue, k, in patch P. These weights would all equal 1.0 in the unbiased case. Patch residues with weights larger than 1.0 have increased importance whereas patch residues with weights less than 1.0 have decreased importance. Proximity weights for the patch residues can be derived from any source of prior information. For example, if the patch represents a binding site of a protein, the weights of the patch residues may be taken from the proximity of the patch residue to the ligand or binding partner. As another example, the patch weights may be derived from mutagenesis results that describe how important each patch residue is to the binding of a substrate or the enzymatic activity of the protein.

In one embodiment, esim(s,P) values, which are specific for a designated patch, P, may be converted to resim(s,P) values, in a manner analogous to equation 6. For a patch of residues:
resim(s,P)=exp((esim(s,P)−esim(template,P))/T) Equation 11c

An additional embodiment of the present invention is the use of patch mode to calculate structure-weighted frequencies for a patch in a manner analogous to the calculation of structure-weighted frequencies for individual residues. In patch mode, the structure-weighted frequencies are the frequencies of finding a certain set of amino acids in the given patch positions. For example, if one is interested in placing both an Ala and an Arg at two positions (the patch) of a protein, higher structure-weighted frequencies found for the Ala and Arg pair than for another pair of amino acids would indicate that the Ala and Arg pair is a more favorable substitution. Equations 3 and 4 are used as before, being slightly modified to reflect the use of the patch of amino acids, as shown in equations 12 and 13.

$$Equation 12 \text{:}$$

$$weight ( s , P ) = h ( s ) * \frac{exp ( esim ) ( s , P ) / T )}{{\sum }_{{s}^{′} = 1}^{N} exp ( esim ( {s}^{′} , P ) / T )}$$

$$Equation 13 \text{:}$$

$$f ( paa , P ) = {\sum }_{s = 1}^{N} weight ( s , P ) \cdot δ ( paa , {paa}_{P}^{s} )$$

where weight(s, P) is the weight of MSA sequence s given reference patch P, paa is the particular patch of amino acids for which a frequency is being determined, paa^{S}p is the patch of amino acids found in MSA sequence s at the positions found in patch P, and f(paa,P) is the structure-weighted patch frequency. As written above, the Kroniker delta function would equal to 1 if all the amino acids in the patch residues of sequence s match all the amino acid for which the frequency is determined. That is, for example, if the user is interest in placing Ala and Arg into two patch positions simultaneously, the sequence weights are added over all sequences in the MSA that have the Ala and Arg at the two patch positions.

The requirement that all amino acids in the sequence s patch are equivalent to all the amino acids for which the frequency is being determined is often overly restrictive. This requirement can be made less restrictive by substituting other functions for the Kroniker delta function. Useful substitute equations include ones that determine the percent identity or percent homology of the two patches, in which similarity matrices like BLOSUM or PAM matrices may again be used.

Precedence scores may also be used with a patch of residues designated. In this case, the precedence score demonstrates that at least one MSA sequence has an environment surrounding the patch that is similar to the environment found in the template sequence. The precedence score is determined in a similar manner to the previous instances in which only one reference residue exists, ie the patch had only one residue. With a patch of many residues:

$$Equation 14 \text{:}$$

$$precedence ( aa , P ) = {Max}_{s ∈ MSA} ( resim ( s , P ) \cdot δ ( aa , {aa}_{i}^{s} ) )$$

Examples of distance-dependent similarity scores or distance-weighted similarity scores in the present invention include the esim values, weights (Eq 3), structure-weighted frequencies, Lscores, resim values, and precedence scores as calculated above. These values may be calculated with one amino acid or a patch of amino acids (a set of amino acids), or other positions used as a reference position in the distance or proximity calculations.

An additional embodiment of the present invention is the calculation of the similarity of the amino acids in the parent patch residues to the amino acids in the patch residues of each related sequence. In prior embodiments, the similarity of the environment in the template sequence to the environment in each MSA sequence was used to judge the fitness of the patch and the environment created by each MSA sequence. Particularly with patches of larger numbers of positions, the similarity of the amino acids in the template patch and each MSA patch may be used to further judge the suitability of a patch and an environment. The similarity of patch residues in the template sequence and each MSA sequence may be calculated by various methods. One method is to sum the similarity scores of the template and MSA sequence amino acids over every position found in the patch, namely

$$Equation 15 \text{:}$$

$$patchsim ( s ) = {\sum }_{p ∈ patch} S ( {aa}_{p}^{s} , {aa}_{p}^{ template} )$$

wherein, s refers to a sequence in the MSA, S refers to the similarity of two amino acids, p is a position in the patch. Alternatively, the analogue of Equation 6 may be used to provide a relative patch similarity measure. These measures of patch similarity can be augmented with terms, for example, that take into consideration the proximity of the patch residue to the environment residues, the overall similarity of the MSA sequences and position- and sequence-specific weighing functions as was done in the comparison of environmental residues described herein. More additions can be incorporated, making the patchsim score more and more similar to the previous environment similarity score.

In short, the designation of which positions are “patch” residues and which are “environment” residues is left to the user. By convention, the algorithm is generally described as calculating the similarity of the environment residues in the template and a MSA sequence. The present invention may be used twice to gain more information useful in the template protein to be designed. A patch may be given as a set of resides and the current invention used to compare the similarity of the environmental positions around the patch. Then, the present invention may be used a second time with the patch residues being defined as those residues in the environment of the first use. Alternatively, the algorithm may be used multiple times with differing definitions of the patch to ascertain the best patch definition or to better judge the relative strengths of the environments presented by the different sequences.

Optimization of Technology of the Present Invention.

In a preferred embodiment, optimal equations and/or parameters for distance-to-proximity conversion, temperature factor, environment similarity, etc. may be selected by systematic evaluation of the effect of equation/parameter choice on the predictive performance of the method. In a preferred embodiment, the present invention may be optimized so that results are in accordance with existing experimental mutational data sets. The parameters of the present invention may include, but are not limited to, the form of the proximity function (equation 1), the proximity scale factor σ (equation 1), the temperature scale T for esim (equations 3 and 6), resim, and the selection of the similarity matrix S (equation 2). In a preferred method, parameters are chosen to maximize the correspondence of amino acid probabilities, log-odds ratio scores, or precedence scores with experimentally determined sequence descriptors that may include stabilities, binding affinities, expression levels, other descriptors, or combinations of sequence descriptors. The correspondence may be measured in various manners including a correlation coefficient, the area under a receiver-operator curve, a P-value, or a Matthew's correlation coefficient.

Virtual MSAs

An additional embodiment of the current invention is the analysis of a virtual MSA generated by automated computational protein design algorithms, such as Protein Design Automation® (PDA®), discussed supra, and Sequence Prediction Algorithm technologies (SPA™ technologies, Raha et al., 2000, Protein Sci 9:1106-1119, U.S. Ser. No. 09/877,695, and U.S. Ser. No. 10/071,859), all incorporated by reference. The application of virtual MSAs is especially important for proteins that have very few natural homologues with which to create a MSA. Computationally generated MSA's may also be generated with unnatural amino acids. In this case, the methods of the present invention may be used to build up, from the virtual MSA, a diverse set of sequences similar to the protein of interest for the given reference position or patch.

Antibodies.

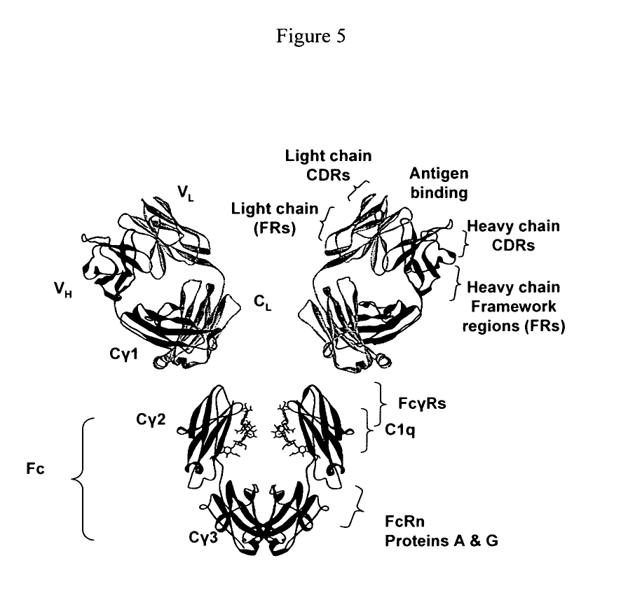

Antibodies bind to specific antigens and consist of two heavy chains and two light chains covalently linked by a disulfide bonds (Janeway, et al. Immunobiology, 2001, 732), incorporated by reference. Both the heavy and light chains contain variable regions, which bind the antigen, and constant regions. The Fc domain, a dimer of a portion of the heavy chain constant regions, is cleaved from the Fab domain upon protease cleavage in vitro. FIG. 5 illustrates a complete IgG antibody and identifies some interaction sites with various proteins.

The variable region of an antibody contains the antigen binding determinants of the molecule, and thus determines the specificity of an antibody for its target antigen. The variable region is so named because it is the most distinct in sequence from other antibodies within the same class, or isotype. The majority of sequence variability occurs in the six complementarity determining regions (CDRs). The variable region outside of the CDRs is referred to as the framework (FR) region. Using the numbering system of Kabat et al. (Kabat, et al., 1991, Sequences and Proteins of Immunological Interest, United States Public Health Service, National Institutes of Health, Bethesda), the CDRs have been defined by various groups. Kabat et al. define the CDRs as light chain residues, 24-34, 50-56, and 89-97 as well as heavy chain residues 31-35B, 50-65, and 95-102, which may be described herein as “Kabat CDRs”. In a preferred embodiment, CDRs may be defined by an analysis of the known three-dimensional structures of antibodies. The atomic positions of light chain residues 27-32, 50-56, and 91-97 as well as heavy chain residues 27-35, 52-56, and 95-102 differ among various antibody structures and may be used as CDRs in the methods of the present invention. Those residues may be referred to as “Xencor CDRs” herein. The methods of the present invention are applicable to both of these CDR definitions of as well as other CDR definitions.

A number of high-resolution structures are available and contain the variable region fragments from different organisms with and with out a bound antigen. The sequence and structural features of antibody variable regions are well characterized (Morea et al., 1997, Biophys Chem 68:9-16; Morea et al., 2000, Methods 20:267-279), incorporated by reference, and the conserved features of antibodies have enabled the development of a wealth of antibody engineering techniques (Maynard et al., 2000, Annu Rev Biomed Eng 2:339-376), incorporated by reference. Fragments comprising the variable region can exist in the absence of other regions of the antibody, including for example, the antigen binding fragment (Fab) comprising V_{H}-Cγ1 and V_{L}-C_{L}, the variable fragment (Fv) comprising V_{H} and V_{L}, the single chain variable fragment (scFv) comprising V_{H} and V_{L} linked together in the same chain, as well as a variety of other variable region fragments (Little et al., 2000, Immunol Today 21:364-370), incorporated by reference.

Commonly used abbreviations for specific regions in an antibody include VL or V_{L}, the variable region of the light chain; VH or V_{H}, the variable region of the heavy chain; CL, the constant region of the light chain; CH, the constant region of the heavy chain; CDR, the complementary determining region; FR, framework region, Fv, the fragment of the variable region; Fc, the fragment of the constant, or “crystalizable” region.

The constant regions of antibodies consist of two or three domains of the heavy chain. In humans, there are five isotypes, or classes, of heavy chains, delta (δ), gamma (γ), mu (μ), alpha (α) and epsilon (ε), giving rise to the IgD, IgG, IgM, IgA and IgE classes of antibodies. The IgA and IgG classes contain the subclasses, IgA1, IgA2, IgG1, IgG2, IgG3, and IgG4. The Fc regions of IgG, IgD and IgA dimerize through their Cγ3, Cδ3, and Cα3 domains, whereas the Fc regions of IgM and IgE dimerize through their Cμ4 and Cε4 domains. The constant regions bind to the Fcγ receptors and are involved in many of the effector functions of antibodies. The methods of the present invention have utility in predicting appropriate protein modifications in all classes of antibodies as well as other proteins.

CDR Grafting.

An embodiment of the present invention is the use of the methods of the present invention in the humanization of antibodies. Humanized antibodies are generally defined as antibodies that have had their variable framework regions and constant regions replaced with human sequences to reduce their immunogenicity in humans. See, e.g. Tsurushita & Vasquez, 2004, Humanization of Monoclonal Antibodies, Molecular Biology of B Cells, 533-545, Elsevier Science (USA): U.S. Pat. Nos. 5,225,539; 5,530,101; 5,585,089; 5,693,761; 5,693,762; 6,180,370; 5,859,205; 5,821,337; 6,054.297; and 6,407,213, all incorporated by reference. One can also convert these regions to sequences contained in the antibodies of other species besides humans, although humans are the most preferred species. For example, converting the sequences in these regions to dog, horse, cat or other sequences may have utility in veterinary medicine. The methods of the present invention provide a means to convert antibody constant and variable framework regions to those of any species.

As is known in the art, a common method of humanizing an antibody is through the process of CDR grafting. In CDR grafting, the CDRs of a donor antibody are combined with the framework regions of an acceptor antibody to create a new antibody. The donor is commonly an antibody whose CDRs bind a particular antigen of interest and is often from an animal such as a mouse, rat or chicken. In a preferred embodiment, the acceptor antibody is a human antibody and commonly a human germline antibody. In this embodiment the CDR graft is used to humanize the donor antibody. The FRs from an antibody from another species, say horse, may be used with the CDRs from the original antibody in a CDR graft to create an novel antibody less immunogenic to horse. This novel antibody may be referred to as the product of equinization, although the terminology is seldom used. The methods of the present invention have utility in selecting the best acceptor antibody from many possible candidates. In a preferred embodiment, the resim scores or other distance-dependent similarity scores are used to select the best acceptor sequence. CDR graft acceptor sequences must be determined for both the heavy and light chains.

A commonly used approach to determining the best human germline CDR acceptor sequence is to select the acceptor sequence with the highest sequence identity or homology to the original donor sequence (Mateo et al. 1997 Immunotechnology 3(1):71-81; Fiorentini et al. 1997 Immunotechnology. 3(1):45-59; Tsurushita et al 2004 Journal of Immunological Methods 295:9-19; Mazor 2005 Molecular Immunology 42(1):55-59, incorporated by reference). Methods such as BLAST or others may be used to determine the sequence identity or homology between two sequences (Altschul, S. F., et al. (1990) J. Mol. Biol. 215:403-410; National Center for Biotechnology, N.I.H. U.S.A., incorporated by reference). Another approach to humanization by CDR grafting uses a consensus sequence derived from the largest light and heavy chain classes, namely VL kappa subgroup I and VH subgroup III (Baca et al. 1997 Journal of Biological Chemistry. 272:16 10678-10684, incorporated by reference). Foote and co-workers have also selected the appropriate acceptor by choosing the acceptor with CDR structures that most closely match the structure of the donor CDRs (Tan et al. 2002 Journal of Immunology 169:1119-1125, incorporated by reference). In most cases, the initial humanized antibody loses some affinity for the antigen and mutation of some framework amino acids back to the original donor, e.g. mouse, amino acid is required to regain antigen affinity.

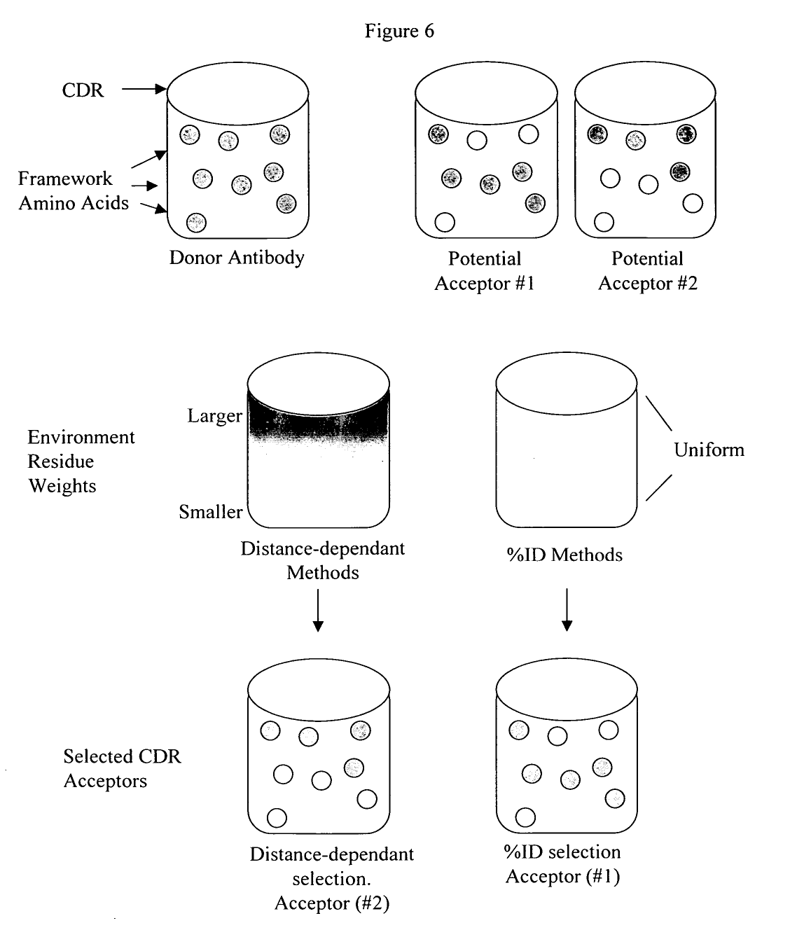

One advantage of the methods of the present invention over methods commonly used in the art is the use of a distance-weighted similarity score to select the best CDR acceptor from a plurality of potential acceptors. The use of a distance-weighted similarity score allows residues closer to the CDRs to have more influence in scoring potential acceptors. This distance-weighing commonly results in different acceptors being selected to receive the CDR graft, whereas methods commonly used in the art allow equal weighing of all residues. Using the example shown in FIG. 6, the methods of the present invention select acceptor #2 even though it has lower sequence identity in the framework regions to the donor antibody framework regions (shown as 4 of 8 filled circles in acceptor #2). In contrast, methods commonly used in the art, which are based on uniform-weight calculations, would select acceptor #1 because of its higher unweighted sequence identity (shown as 5 of 8 filled circles in acceptor #1).

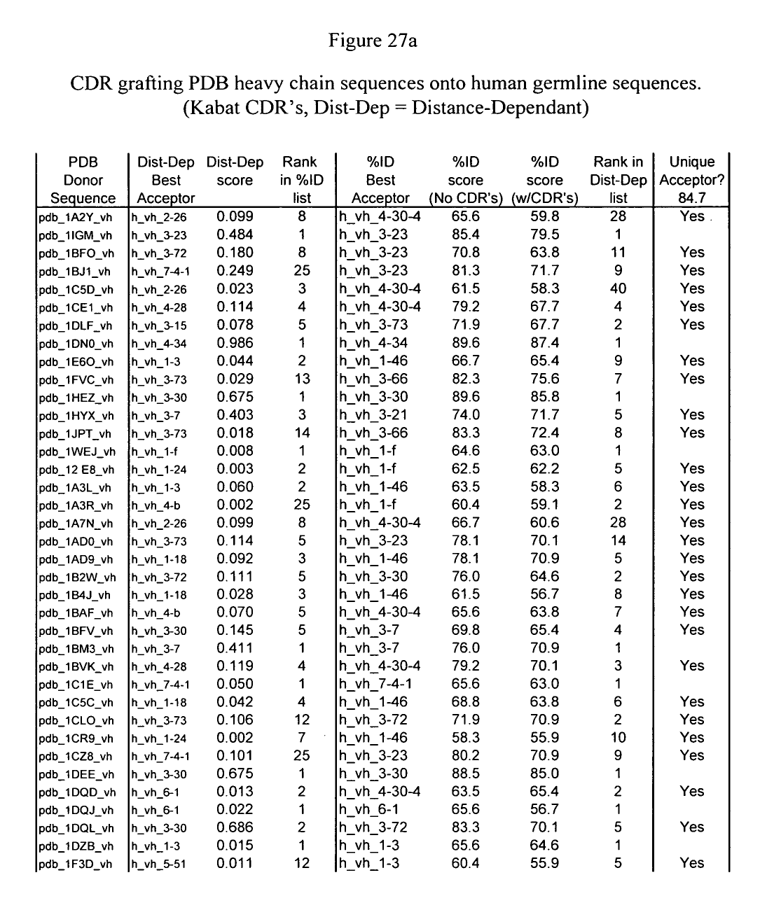

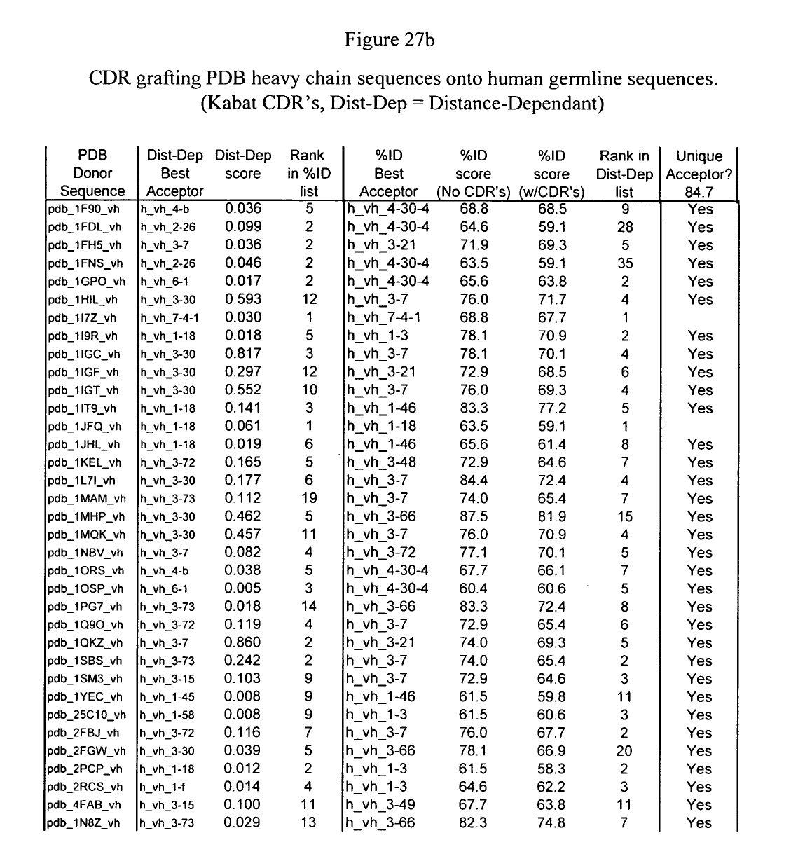

CDR graft acceptors may be determined for both the heavy and light chains, although the two procedures are analogous. For example, to use the methods of the present invention in grafting the heavy chain CDRs from a rat antibody to the framework regions of a human antibody, an alignment of a plurality of human germline heavy chain sequences may be used as well as a representative antibody structure. The CDRs may be Kabat CDRs, Xencor CDRs or another definition of CDRs with Xencor CDRs being the most preferred. The alignment includes all potential acceptor sequences, preferably the human heavy chain germline sequences, and may be created by clustalW, BLAST (NCBI, NIH) or other sequence alignment algorithms commonly known in the art. The representative structure may be any of those listed in FIG. 27 or any other heavy chain structure such as those in the Protein Data Bank (PDB).

In a preferred embodiment of using the methods of the present invention for CDR grafting, the heavy chain CDRs, e.g., are defined by the user as the patch of residues and the patch mode analysis is done. Patch mode calculates the suitability of each environment, ie the framework residues, as potential acceptors. The suitability of each sequence, or environment more specifically, may be judged by a distance-weighted similarity score of the present invention. For example, higher resim scores of acceptor sequences demonstrate the improved fitness of those acceptors for the graft. Whereas in this, or any, use of patch mode, any set of residues can be defined as the patch, defining the CDRs as the patch is preferred over defining the framework regions as the patch. Light chain sequences are also processed in a similar manner.

Comparison of CDR Grafting with Distance-Dependant Methods of the Present Invention and Percent Identity Methods.

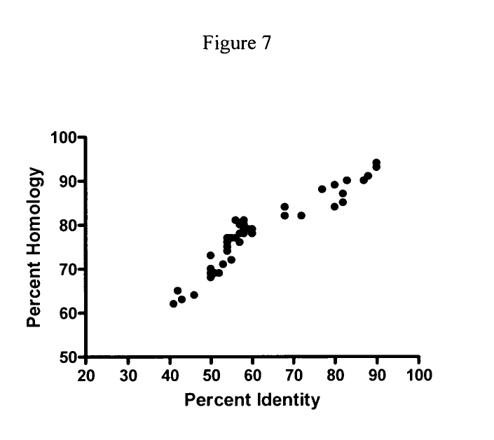

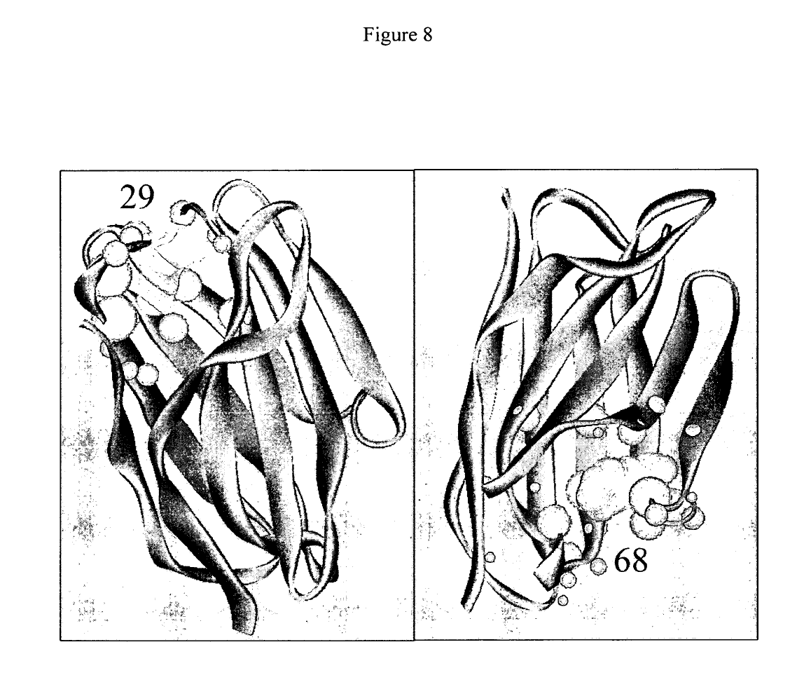

As is know in the art, a common group of methods for choosing the best human sequences to accept the CDRs is to choose the human sequence with the highest percentage of identical amino acids to the original variable region. These methods are referred to herein as the percent identity (% ID) methods. See, for example, PDL. The percent similarity is also commonly used in the art to judge the fitness of potential acceptors. An often-used measurement of percent similarity is the “percent positives” score as calculated by BLAST (National center for Biotechnology Information, National Institute of Health, USA). The percent identity and percent similarity measures give very similar results as shown in alignments of the human germline heavy chain 1-2 to 52 other human germline heavy chain sequences (FIG. 7).

Generally, the methods of the present invention do not select the same human sequence for a best acceptor as do the % ID methods. (See below for examples using donors from the PDB antibodies and mouse germlines.) This difference is due to the unique aspects of the present invention, including but not limited to, the use of the protein structure to generate a distance-dependent similarity score. The resim scores of the present invention include the use the proximity of each residue to the CDRs in determining the importance of that position to the overall score. The % ID methods, or percent homology methods, give equal importance to all positions, thereby disregarding the larger influence of some residues over other residues.

Methods in the art (e.g. those of Queen, etc.) generally teach the use of back-mutations to repair the affinity lost upon CDR grafting into a chosen human acceptor. However, using the methods of the present invention, fewer back-mutations will generally be required, leading to a more efficient and cost-effective CDR-grafting process. This is a direct consequence of the fact that distance-dependant methods are designed to select acceptors with frameworks that are more similar at positions proximal to CDR positions.

### EXAMPLE 1

Human Heavy Chain Sequences

The antibody heavy chain sequences were be aligned and used with an existing structure as input into the present invention. FIG. 8 shows the structure of Herceptin® (trastuzumab) (Genentech/Biogenldec) (PDB code 1FVC) and proximity values determined by an embodiment of the present invention. The left panel shows proximities values determined when position 29 is designated as the reference position or patch. The amino acid of position 29 in the reference structure is shown as a non-spherical surface. The remaining positions in the protein, the environment positions, are shown as a spheres positioned on their CA positions in the structure. The volumes of the spheres are proportional to their proximities to position 29. Larger sphere indicate more proximal environment positions, which are weighted more strongly in the determination of the structure-weighted frequency, resim and precedence scores. The right panel shows the proximity values determined when position 68 is the patch, or reference, position.

### EXAMPLE 2

Sequence Weight Determination

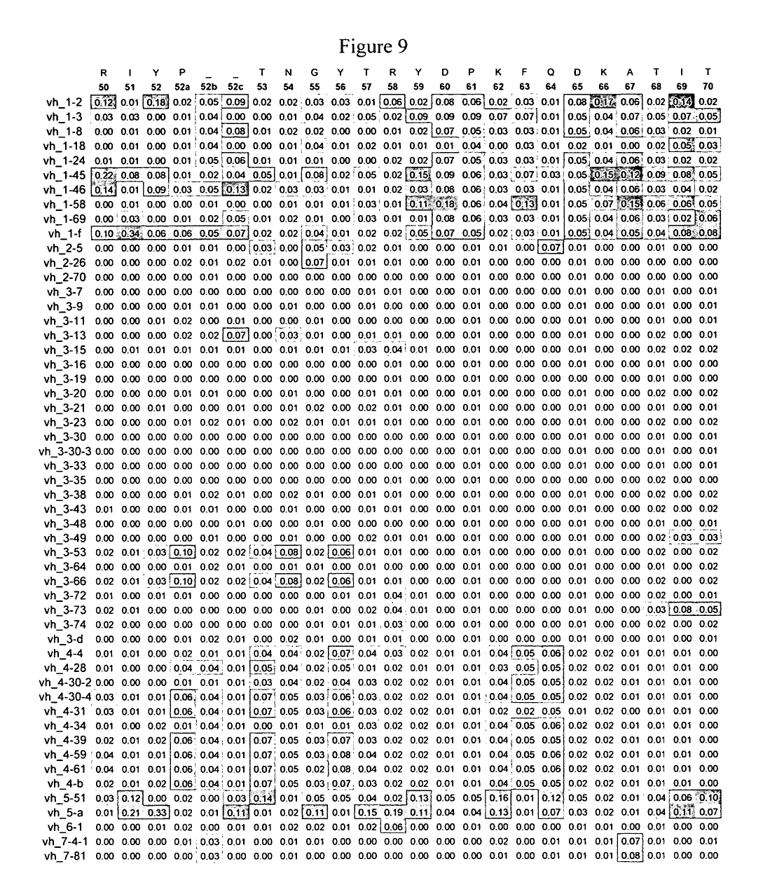

An alignment of human heavy chain germline sequences, the reference sequence, m4D5, and the structure, PDB code 1FVC, was used to determine the sequence with the most suitable environment around each position in the multiple sequence alignment (MSA). FIG. 9 shows the sequence weights calculated with equation 3 using a temperature (T) value of 1, the BLOSUM62 (Henikoff J. G. Proc. Nat Acad. Sci USA 89:10915-10919 (1992), incorporated by reference) similarity matrix (eq. 2) and a δ value of 5 (eq 1). FIG. 9 illustrates how the similarity of each sequence to the reference sequence depends upon the position given as a reference position. For example, the environment around position 50 is the most similar (similarity score=0.22) in sequence vh_{—}1-45 to the reference environment of all the listed sequences.

### EXAMPLE 3

Patch Mode—Multiple Residues Considered.

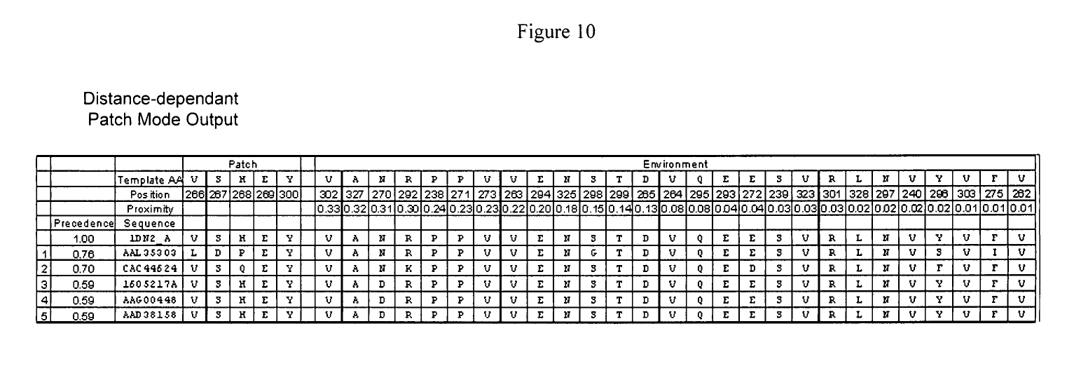

The methods of the present invention are useful in patch mode to determine the best environment in which to place a patch of amino acids, or to determine the best patch of amino acids to place into a particular environment. A template structure and a multiple sequence alignment comprising the sequence of the template structure are input as are a list of residue positions defining the patch. FIG. 10 shows the distance-dependant resim scores determined using a multiple sequence alignment of antibody Fc domains and an Fc structure, PDB code 1DN2. The multiple sequence alignment used was generated with BLAST (Altschul, S. F., et al. (1990) J. Mol. Biol. 215:403-410, incorporated by reference) using the sequence of the human IgG1 Fc domain as input. The multiple sequence alignment contained 249 positions (residues plus gaps) and 137 sequences including the sequence of the template structure. Henikoff weights (Henikoff & Henikoff, 1992, Proc. Nat. Acad. Sci. USA 89: 10917, incorporated by reference) were applied to the sequences to reduce the influence of very similar sequences. User-defined position specific weights were not used, allowing the proximity values to determine the contribution of each environment residue to the environment.

For this example, a patch was chosen using residues 266, 267, 268, 269 and 300. The 27 environment residues closest to the patch residues are shown with their proximity values on the right side of the figure. V302 is the closest environmental residue to the patch having a proximity value of 0.33. The top 5 sequences with the best environment for the patch are shown under the sequence of the template structure. These sequences gave a high precedence score. The top ranking sequence, labeled “AAL35303”, has an environment that differs from the environment from the template sequence in that it contains a Gly at position 298 in place of a Ser. This change, and the other less proximal changes, drops the precedence score below the value of 1.0, which is found only in an exact match.

### EXAMPLE 4

CDR Grafting.

One example of the use of the current invention is found in CDR grafting. In CDR grafting, the complement-determining regions (CDRs) of the variable region of an antibody, say a murine antibody, are substituted into another antibody, say a human antibody. This procedure produces an antibody that possesses the antigen-binding specificity of the murine antibody and has human-derived sequences in the remaining positions to reduce the stimulation of an immune response in human patients. In the case of the antibody heavy chain, the researcher must decide which of the many possible human heavy chain sequences would be the best choice to accept the graft of the murine CDRs. Choosing a compatible human heavy chain acceptor will minimize the losses in antigen binding affinity, which frequently accompany CDR grafting.

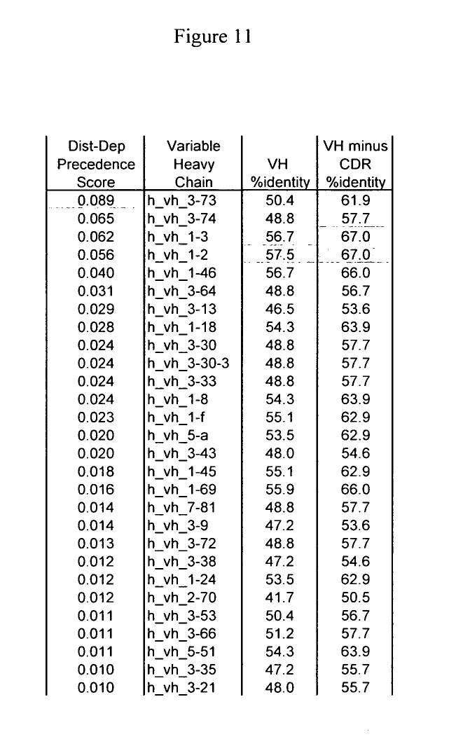

FIG. 11 shows the compatibility of the CDRs of a murine heavy chain antibody used by Carter et al. (1992 Proc Nat. Acad. Sci. USA 89(10):4285-9, incorporated by reference) with many possible acceptor human antibody germline sequences. Surprisingly, the methods of the present invention demonstrate that the most compatible human sequence is that of h_vh_{—}3-73 (using resim scores), whereas other human sequences, namely h_vh_{—}1-2 and h_vh_{—}1-3, would be chosen based on the percent sequence identity. The percent sequence identity measure identifies the human sequence that is most similar to the murine sequence overall. The methods of the present invention, however, include the structure in the analysis and identify the human sequence that is most similar to the murine sequence in the regions proximal to the CDRs.

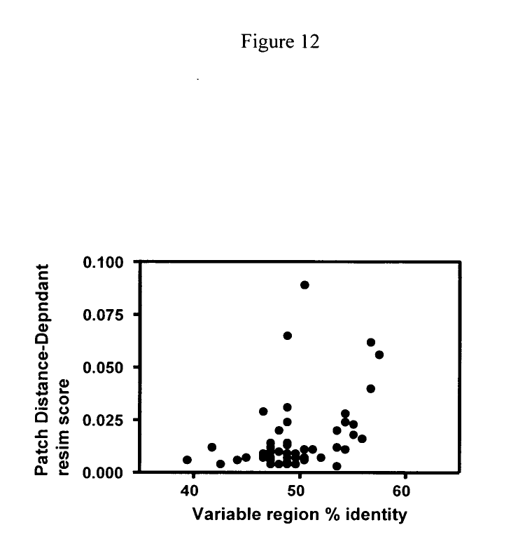

FIG. 12 shows the poor correlation of the resim scores and the overall percent identities of the human sequences to the murine sequence. (In this graph the percent sequence identity was calculate using all residues in the variable heavy domain. Using only those residues not found in the CDRs produces a similar graph, as predicted from the strong correlation of columns 3 and 4 in FIG. 11. The two human sequences with the highest resim scores, h_vh_{—}3-73 and h_vh_{—}3-74, have percent identities that are not significantly above the average percent identity for the 52 human sequences. Therefore the methods of the present invention suggest a human acceptor sequence that is not the optimum sequence as judged by the overall similarity of the murine sequence to the human sequences.

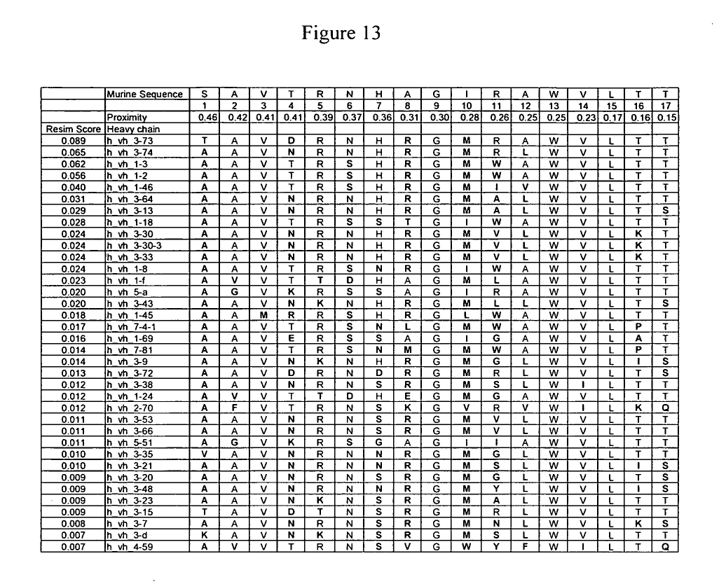

By looking at the environment residues most proximal to the CDRs, the residues in h_vh_{—}3-73 are identified so that gives it a favorable resim score for accepting the murine CDRs. FIG. 13 shows the human heavy chain sequences and their resim scores. Each column of the table shows the amino acid found in one environment position and the position's proximity to the CDR patch. The closest environment residue, which is Ser in the murine sequence, has a proximity of 0.46 from the patch. The most favorable heavy chain sequence, h_vh_{—}3-73, has Thr at this position whereas most other heavy chain sequences have Ala at this position. h_vh_{—}3-73 has a more favorable resim score than say, h_vh_{—}3-74, because Thr is a more conservative substitution for Ser than is Ala. Amino acid differences at other positions also influence the resim scores, but those differences are weighted less because of their lower proximity.

### EXAMPLE 5

Grafting the CDRs of PDB structure 1C5D.

Distance-dependant methods of the present invention were used to select the best acceptor heavy and light chain sequences from the human germline. For the distance-dependant calculations, the PDB structure 1SBS was used as template structure and multiple sequence alignments were created for the heavy and light chains. The multiple sequence alignment for the heavy chain included 53 human germline heavy chain sequences each containing 127 positions (amino acids plus gaps). The light chain alignment contained 45 human germline sequences and 115 sequence positions. The alignments were created with clustalW (Jeanmougin,F. et al (1998) Trends Biochem Sci, 23, 403-5, incorporated by reference) and adjusted manually in some positions to improve the alignment.

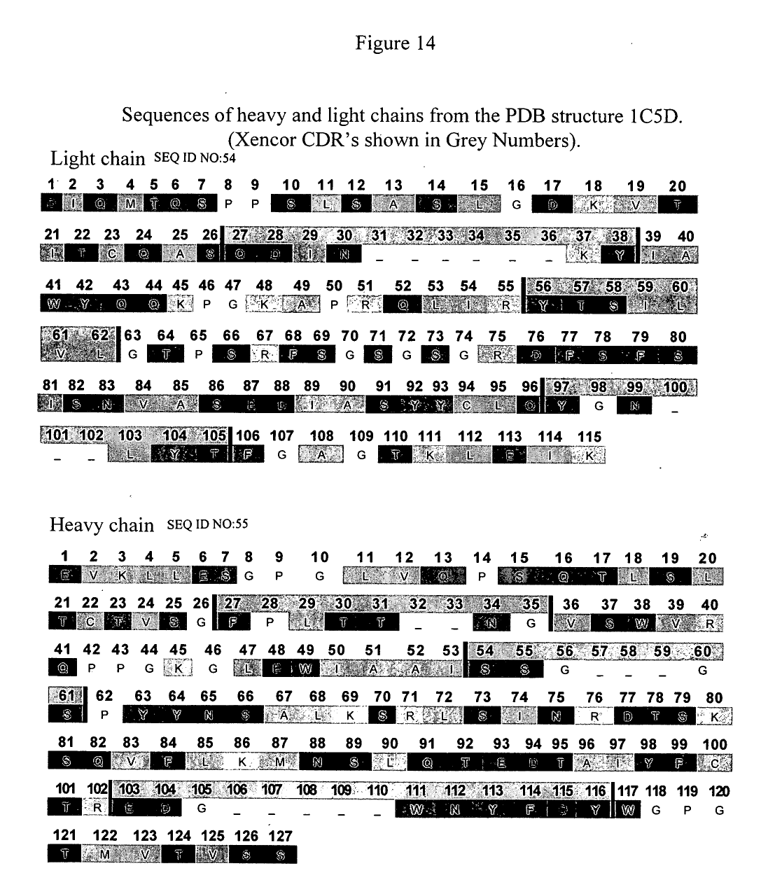

Proximities were calculated using Eq 1, Eq 11 and a σ parameter of 5.0. Patch esim values (Eq 10) were calculated with the BLOSUM62 substitution matrix. Position-specific sequence weights were calculated with Eq 12, using a temperature of 3.0 and Henikoff sequence weights (Henikoff S and Henikoff H. G. Proc Natl Acad Sci USA. 1992 Nov. 15;89(22):10915-9, incorporated by reference). For the heavy chain predictions, the heavy chain from 1C5D was used as a donor or template protein with Xencor CDR positions defined as the patch (positions 27-35, 52-56, and 95-102 as numbered in Kabat et al. or equivalently positions 27-35, 54-61, and 103-116, using the numbering convention of its multiple sequence alignment shown in FIG. 14). For the light chain predictions, the light chain from 1C5D was used as a donor or template protein again with Xencor CDR positions defined at the patch (positions 27-32, 50-56, and 91-97 as numbered in Kabat et al. or equivalently, positions 27-38, 56-62, and 97-105, using the numbering convention of its multiple sequence alignment shown in FIG. 14). The resim value of each sequence in the MSA is used to judge the fitness of the sequence as the graft acceptor, with higher values indicating better acceptors.

The best acceptor for the 1C5D light and heavy chain CDRs according to distance-dependant methods of the present invention are the human germline light chain 2-26, h_vkl_{—}2-26, and the heavy chain 1-17, h_vh_{—}1-17. As is known in the art, one could also chose the best acceptor sequences based on the overall sequence identity of each germline sequence to the donor sequence. Using this method, the % ID method, would result in the selection of kappa light chain 1-33, h_vlk_{—}1-33, and heavy chain 4-304, h_vh_{—}4-30-4, as the best acceptor sequences.

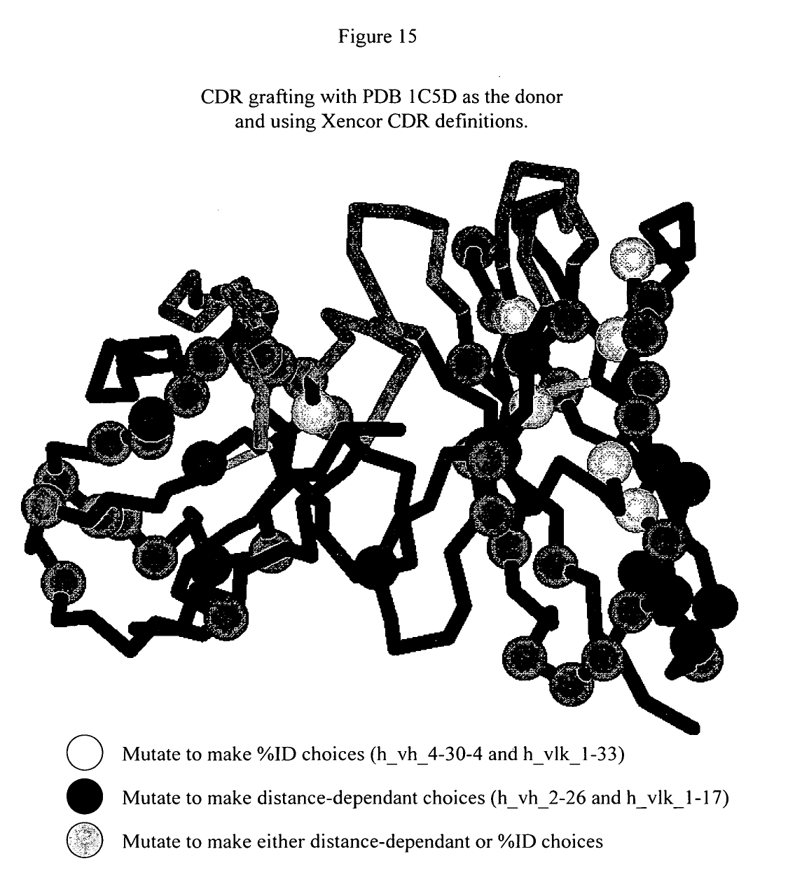

FIG. 15 shows the mutations that are needed in the 1C5D framework regions to produce the humanized antibodies selected from each method. The 1C5D backbone is shown in blue in the framework regions and in orange in the CDR regions. Residues at positions shown in purple (11 residues) must be mutated to create the acceptor frameworks chosen by the distance-dependant methods of the present invention, h_vkl_{—}2-26 and h_vh_{—}1-17, but do not need to be mutated to create the framework regions selected by the % ID method, h_vlk1-33 and h_vh_{—}4-30-4. Residues at positions shown in white (7 residues) must be mutated to create the acceptor framework chosen by the % ID method, but not the framework chosen by the distance-dependant methods of the present invention. Residue in green must be mutated to create the best acceptor chosen by both methods.

As expected, more mutations are required (red plus green positions) to create the acceptor chosen by the methods of the present invention than are required (white plus green positions) to create the acceptor chosen by the % ID method. The % ID method, by definition, chooses the acceptor sequence requiring the least number of mutations. The distance-dependant methods, however, chose acceptor sequences that have fewer and more conservative mutations near in space to the CDRs. The distance-dependant humanization product, therefore, is less likely to disrupt the structure and function of the CDRs, which cause decreases in antigen-binding affinity.

### EXAMPLE 6

Grafting the CDRs of PDB Structure 1IGC.

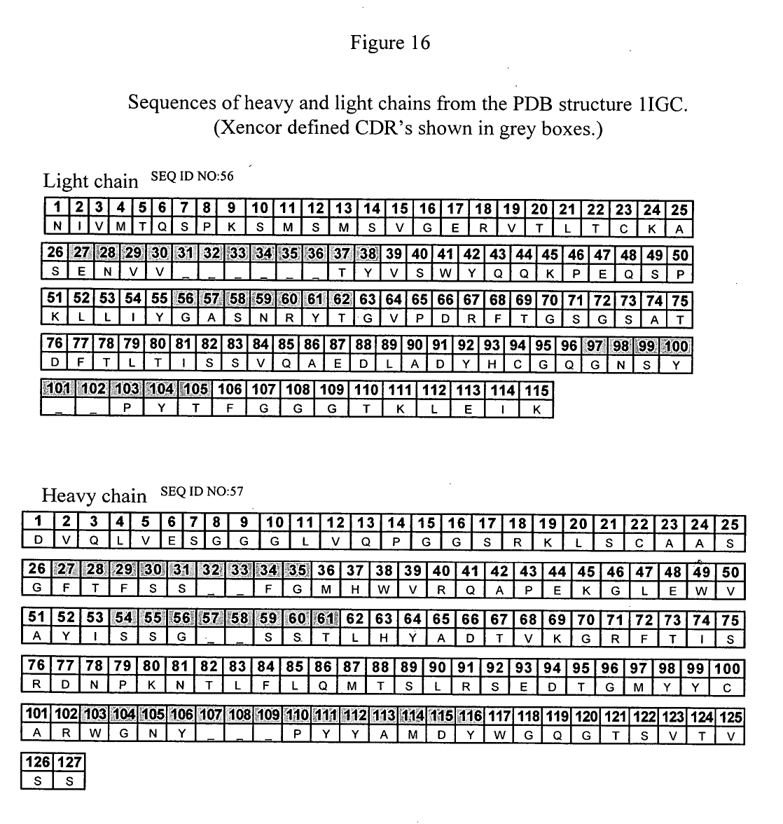

The distance-dependant methods of the present invention were used to graft the CDRs from the PDB structure 1IGC into the best human germline sequence. The most preferred CDR acceptor was also chosen using the overall sequence identity, in a similar fashion to the above example with PDB structure 1C5D. The same parameters and Xencor-defined CDRs were used. FIG. 16 shows the heavy and light chain sequences from PDB 1IGC and their numbering in their multiple sequence alignments used by the methods of the present invention. The distance-dependant methods of the present invention identified h_vh_{—}3-30 and h_vlk_{—}3D-7 as the best heavy and light chain CDR acceptors respectively. Using the overall percent identity, h_vh_{—}3-48 and h_vlk_{—}1D-16 are selected as the best CDR acceptors.

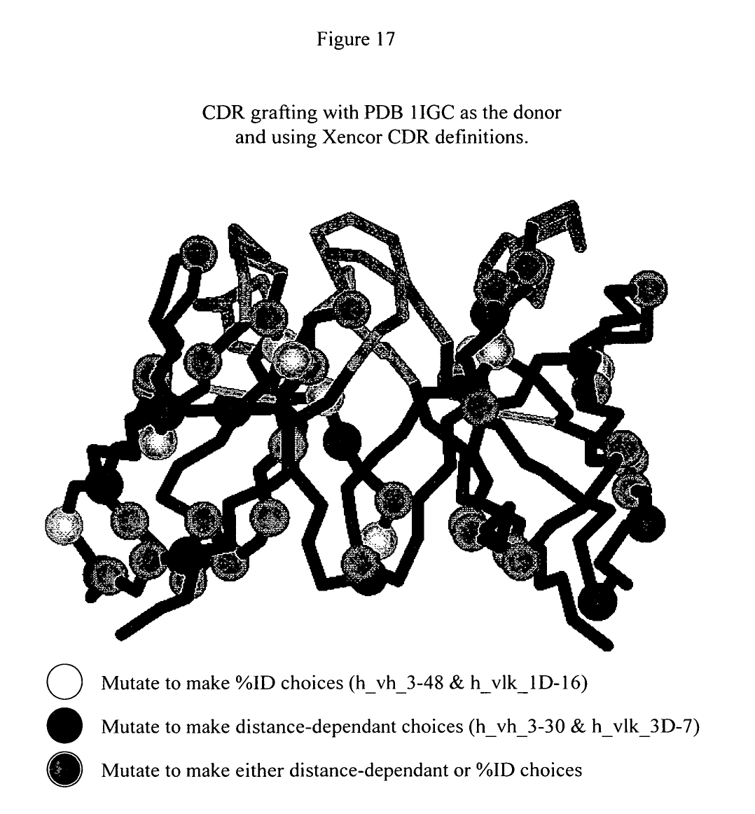

The residues colored in green in FIG. 17 must be mutated in 1IGC to create the humanized antibodies selected by both the methods of the present invention and the percent identity method. Additionally, the residues in purple must be mutated to create the humanized antibody chosen by distance-dependant methods, but are not necessary to mutate to create the humanized antibody chosen by the percent identity method. In contrast, the residues in white must be mutated to create the humanized antibody chosen by the percent identity method, but are not necessary to mutate to create the humanized antibody chosen by the distance-dependant methods. The percent identity method forces more mutations to be made near the CDR regions (Orange backbone), which are likely to affect the antigen-binding affinity of the antibody. The distance-dependant methods of the present invention suggests more mutations overall, but the mutations are more distant from the CDR's and less likely to affect antigen binding.

### EXAMPLE 7

CDR Grafting of m4D5.

m4D5 is a mouse antibody against Her2, a cell surface protein whose over-expression is correlated with some breast cancers. A humanized product of m4D5, called trastuzumab (Herceptin®, Genentech), is currently marketed to breast cancer patients. A humanized m4D5 using the methods of the present invention and the same parameters as in EXAMPLE 5 were designed. For the light chain, the top-scoring human germline acceptor is kappa chain 4-1, h_vlk_{—}4-1, with a resim score of 0.0163. Overall, the germline sequence with the highest percent identity to m4D5 is h_vlk_{—}1-33, having 80 identical residues from, or 80/115=69.6% identity to, m4D5. This germline, h_vlk_{—}1-33, is the 11^{th} best acceptor according to the methods of the present invention. For the heavy chain sequence, human germline acceptor 3-73, h_vh_{—}3-73, was selected by the methods of the present invention as the best acceptor with a resim score of 0.0893. Over the entire variable region, the germline heavy chain with the highest identity to m4D5 was 1-2, h_vh_{—}1-2, containing 102 identical residues to m4D5 in 127 sequences positions, or 102/127=80.3% sequence identity. This acceptor, h_vh_{—}1-2, ranked 4^{th} of the 53 potential human germline acceptor sequences as determined by the methods of the present invention.

The heavy chain acceptor chosen by the distance-dependant methods of the present invention, h_vh_{—}1-2, requires 31 mutations to be made to m4D5 in the 96 framework positions. For comparison, tratuzumab required 32 heavy chain mutations from m4D5 in these 96 framework positions. Some of these changes were necessary because the original grafting of the m4D5 CDR's onto a human acceptor sequence resulted in diminished antigen-binding affinity. Phage display was used to find mutations that helped regain antigen binding (Gerstner et al (2002) Journal of Molecular Biology 321, 851-862).

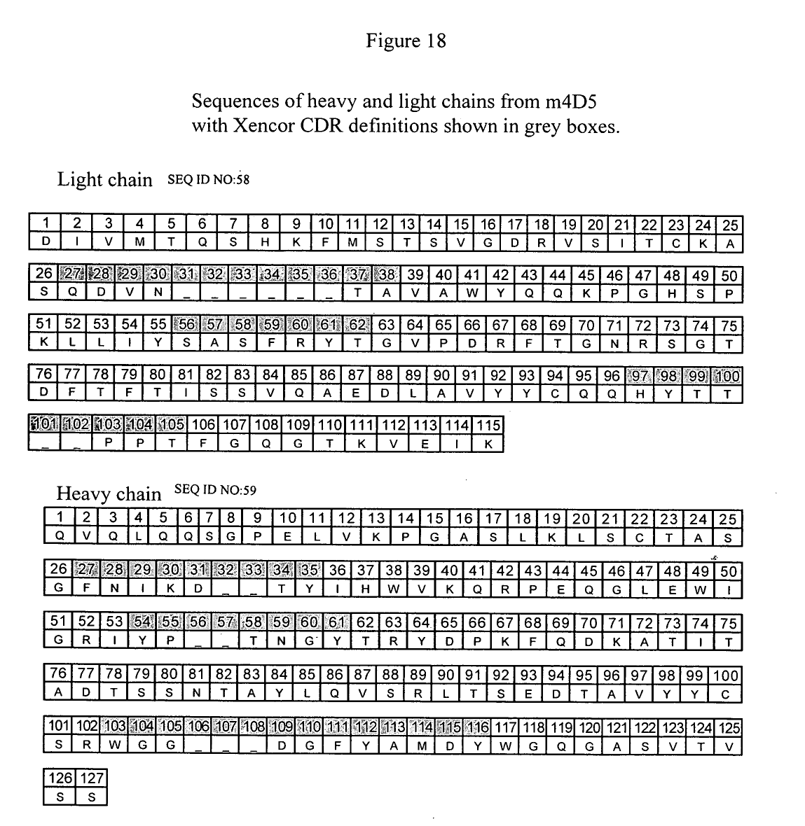

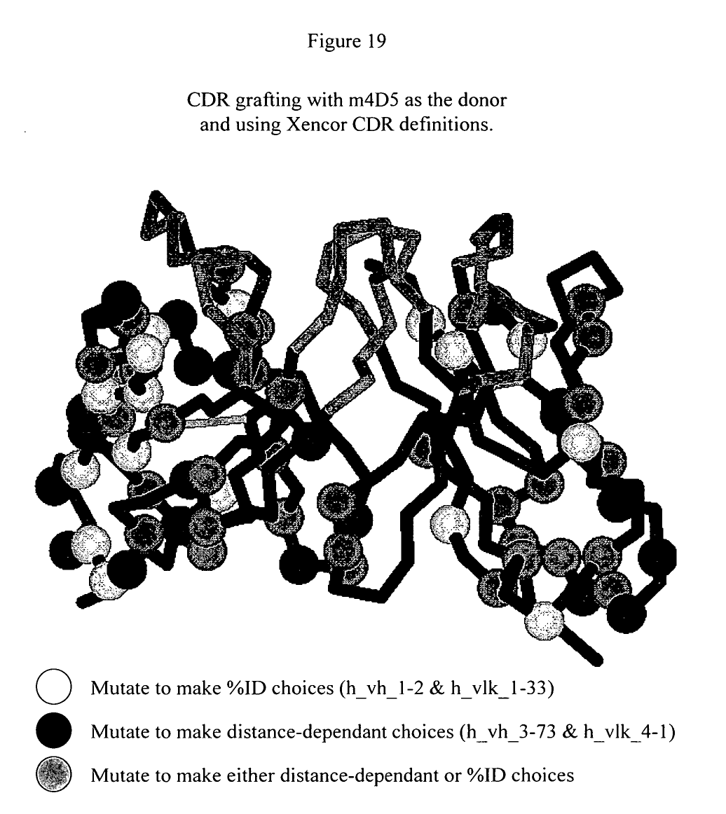

The sequence of m4D5 and its numbering in the Xencor-numbered heavy and light chain alignments are shown in FIG. 18. The positions in m4D5 that are changed during humanization to h_vlk_{—}4-1 and h_vh_{—}3-73, i.e., the positions changed in creating the distance-dependant humanized antibody, are shown as green and purple CA atoms in FIG. 19. Likewise, the positions in m4D5 that are changed during humanization to h_vlk_{—}1-33 and h_vh_{—}1-2, ie the positions changed in creating the % ID humanized antibody, are shown as green and white CA atoms in FIG. 19. Fewer mutations near the CDR regions, particularly in the light chain (generally, the right side of FIG. 19), are required to create the distance-dependant-designed humanized antibody.

Beginning with the m4D5 sequence of FIG. 18, the following mutations are all made substantially simultaneously in the heavy chain framework regions to create the humanized heavy chain chosen by the methods of the present invention: Q1E, Q5V, Q6E, P9G, E10G, K13Q, A16G, T23A, I36M, K40R, R42A, P43S, E44G, Q45K, I50V, R63A, D65A, P66A, K67S, F68V, Q69K, D70G, K71R, A72F, T75S, A76R, T78D, S80K, V87M, S88N, R89S, T91K, S92T, S101T, A121T, and S122L. Likewise, the following mutations were all made substantially simultaneously in the light chain framework regions to create the humanized light chain chosen by the methods of the present invention: H8P, K9D, F10S, M11L, S12A, T13V, V15L, D17E, V19A, S20T, T22N, A25S, V39L, H48Q, S49P, T69S, N71S, R72G, F79L, V84L, and L89V.

### EXAMPLE 8

CDR Grafting AC10.

A humanized AC10 using the methods of the present invention and the same parameters as in EXAMPLE 5 was designed. For the light chain, the top-scoring human germline acceptor is kappa chain 1-39, h_vlk_{—}1-39, with a resim score of 0.05516. Overall, the germline sequence with the highest percent identity to AC10 is h_vlk_{—}4-1, having 79 identical residues from, or 79/115=68.7% identity to, AC10. This germline, h_vlk_{—}4-1, is the 18^{th} best acceptor according to the methods of the present invention. For the heavy chain sequence, human germline acceptor 1-18, h_vh_{—}1-18, was selected by the methods of the present invention as the best acceptor with a resim score of 0.1303. This acceptor, h_vh_{—}1-18, requires 26 mutations to be made to AC10 in the 96 framework positions. Over the entire variable region, the germline heavy chain with the highest identity to AC10 was 1-3, h_vh_{—}1-3, containing 83 identical residues to AC10 of 127 sequences positions, or 83/127=65.4% sequence identity. This acceptor, h_vh_{—}1-3, ranked 4^{th} of the human germline acceptor sequences as determined by the methods of the present invention.

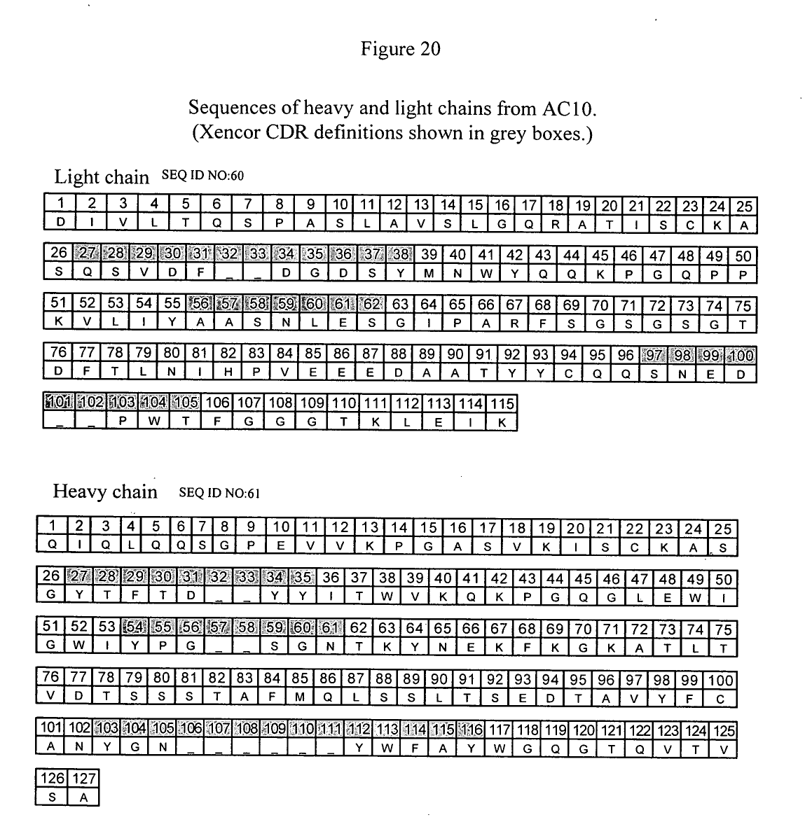

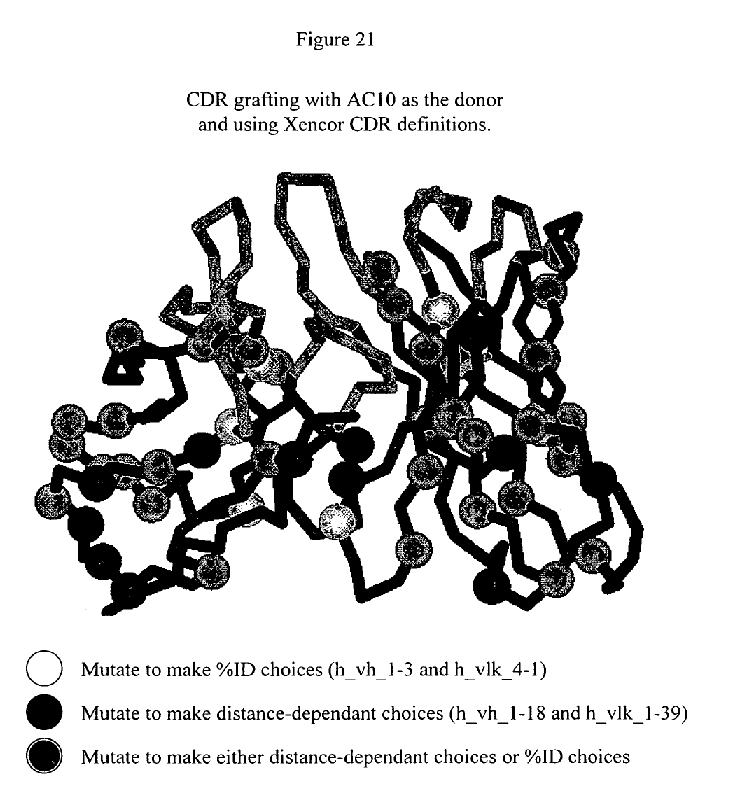

The sequence of AC10 and its numbering in the heavy and light chain alignments are shown in FIG. 20. The positions in AC10 that are changed during humanization to h_vlk_{—}1-39 and h_vh_{—}1-18, i.e., the positions changed in creating the distance-dependant humanized antibody, are shown as green and purple CA atoms in FIG. 21. Likewise, the positions in AC10 that are changed during humanization to h_vlk_{—}1-3 and h_vh_{—}4-1, ie the positions changed in creating the % ID humanized antibody, are shown as green and white CA atoms in FIG. 21.

Starting with the original AC10 heavy chain as shown in FIG. 20, the following mutations are made in the framework regions to convert the AC10 heavy chain to the humanized AC10 heavy chain with the human germline h_vh_{—}1-18 as the acceptor: 12V, Q5V, P9A, V12K, I20V, T37S, K40R, K42A, I50M, K63N, N65A, E66Q, F68L, K69Q, K71R, A72V, L74M, V76T, S80T, F84Y, Q86E, S88R, T91R, E93D, F99Y, N102R, Q122L, and A127S. Likewise, the following mutations are made substantially simultaneously in the light chain framework regions to create the humanized light chain chosen by the methods of the present invention: V3Q, L4M, A9S, A12S, V13A, L15V, Q17D, A19V, S22T, K24R, M39L, Q48K, P49A, V52L, I64V, A66S, N80T, H82S, P83S, V84L, E85Q, E86P, and A89F.

### EXAMPLE 9

Patch Position-Specific Weights.

Position-specific weights may be incorporated into the algorithms to emphasize the influence of certain patch residues over others. These weights are used as w(k) in Eq. 11b. Weights may be determined from any prior information about which patch residues are more or less important. For example, if the patch is the CDR of an antibody during the acceptor selection of CDR grafting, the relative importance of each patch residue to antigen binding could be used as the w(k) weights. The relative importance of each CDR residue may be determined from the mutant effects on antigen binding affinity, from CDR residue distances to the antigen in a structure, from antigen-binding frequencies of CDR residues (e.g. MacCallum (1996) Journal of Molecular Biology 262:732-745; Ramirez-Bentitez (2001) Proteins: Struc. Func. and Genet. 45:199-206, all incorporated by reference) or any other measure of the CDR residue's importance.

Position-specific weights were incorporated into the EXAMPLE 8 determination of the best heavy chain acceptor of a CDR graft from the murine antibody AC10. The CDR regions were used as the patch and the same distance-dependant parameters were used as in EXAMPLE 8, except the use of Eq 11b and its position-specific weights, w(k). Residue N61, the last residue in CDR2, was emphasized by keeping its weight at 1.0 and setting the other CDR residue weights to 0.125. (Alternatively, residue N61 could have a weight of 8.0 with the other CDR residue weights being 1.0. The final patch resim scores change in magnitude, but the order of potential acceptors remains identical. Residue numbering is that of FIG. 20.) The proximities of the environment residues to N61 are now 8-fold higher than in the unbiased case. Therefore environment residue near N61 will have a larger influence in determining the similarity of a potential acceptor's environment to AC10's environment.

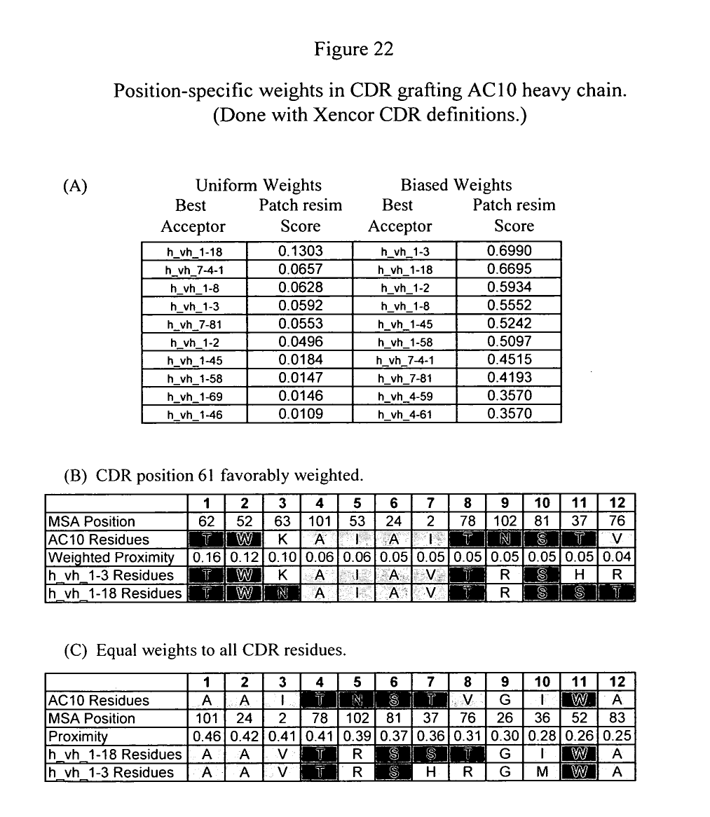

In the unbiased case, the best human germline acceptor for AC10's heavy chain CDR's is 1-18, h_vh_{—}1-18. With this additional weight on residue 61, the best acceptor is now human germline heavy chain 1-3, h_vh_{—}1-3. FIG. 22a shows the top 10 choices of human germline heavy chain acceptor in the unbiased case and for the case where the CDR residues are biased to give N61 8-fold more importance.

The human germline h_vh_{—}1-3 is now favored over h_vh_{—}1-18 as the best acceptor largely from the influence of the environment residue, 63. As shown in FIG. 22(b), position 63 is the 3^{rd} most important environment residue having a weighted proximity to the patch of 0.10. The germline 1-3 has a Lys at MSA position 63, which is a perfect match to the Lys in the AC10 reference sequence. The germline 1-18, however, has an Asn at position 63, which is a less favorable match. Germline 1-18's less preferred Asn at this influential position lowers its patch resim score below that of germline 1-3 demonstrating that 1-18 is a less suitable acceptor sequence. At the two strongest environment positions, namely 62 and 52 with weighted proximities of 0.16 and 0.12 respectively, both germlines have identical amino acids to those of the AC10 donor sequence and therefore these positions do not help distinguish these two germlines from each other.

For comparison, the top 12 proximities in the equal-weighted case are shown in FIG. 22c. With all CDR weights equaling 1.0, environment residue 63 is no longer a particularly influential residue and does not appear in the top 12 most proximal residues. (Residue 63 now has the 19^{th} highest proximity.) Instead, positions 37 and 76 are those that distinguish germlines 1-18 and 1-3. Clearly, germline 1-18 is favored in the unweighted case. Germline 1-18's Ser and Thr at these two positions are much more conservative substitutions for the donor's Thr and Val than are germline 1-3's His and Arg. In fact, with equal weights, germline 1-3 drops to the 4^{th} best acceptor for AC10's heavy chain CDRs.

### EXAMPLE 10

Binary vs Continuous Proximities.

As described herein, the proximities of a residue in the environment of a patch of interest, e.g. a CDR in an antibody, may be calculated by various means. Calculating proximities as being inversely proportional to distance between the patch and the environment residue is a preferred embodiment of the present invention. Proximities calculated in this manner, for example using the guassian function in Equation 1, are a continuous function of the distance. Proximities may also be calculated in a simpler, binary manner. For example, environment residues within a certain distance of the patch (say, 8.0 Angstroms) are given proximities of 1.0 (full weight) whereas environment residues outside of this distance are given proximities of 0.0 (no weight) and are essentially dropped out of the calculation. Assigning binary proximities is equivalent to deciding which environment residues will be used and giving all environment residues equal weight, independently of the residues distance to the patch. Other methods of calculating proximities from distances are non-continuous, such as assigning high, medium, and low proximities (or weights) to the environment residues.

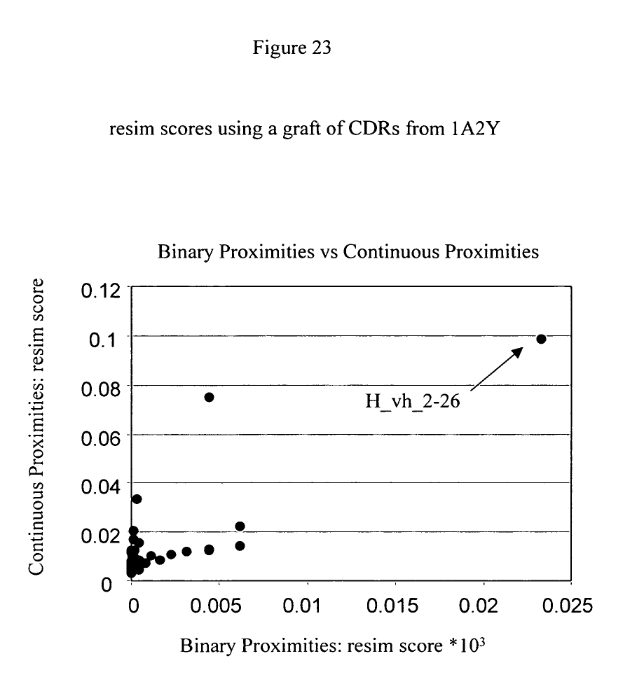

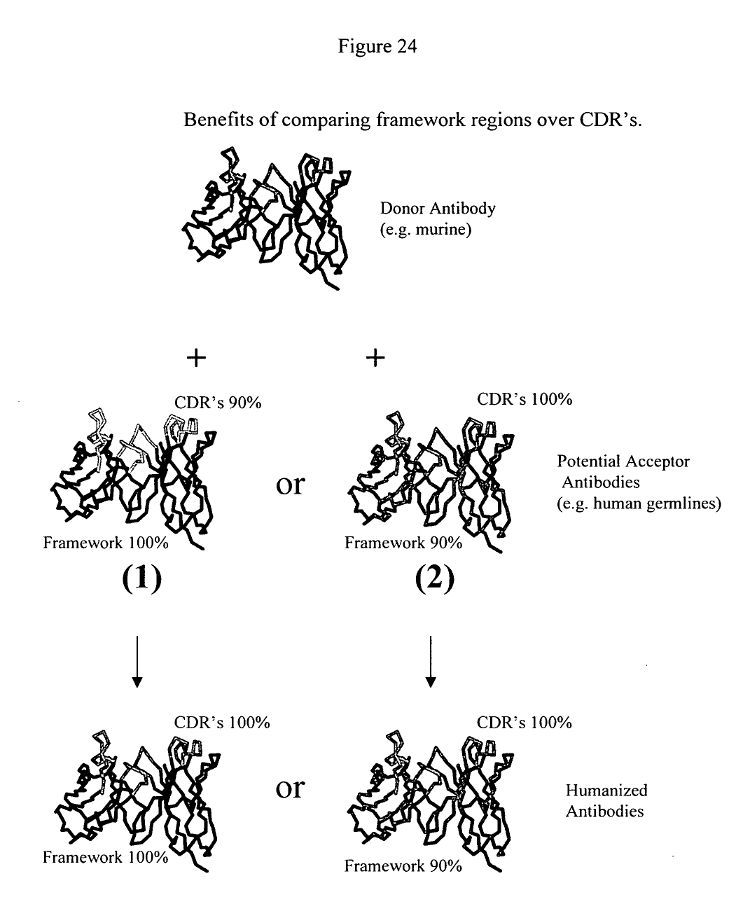

The best CDR acceptor for the donor antibody from the PDB structure 1A2Y using both continuous and non-continuous proximity calculations is h_vh_{—}2-26 (FIG. 23). The heavy chain from 1A2Y was the CDR donor and 53 human germline heavy chain sequences were used as possible acceptors. The CDRs used were those defined by Kabat et al and the distance-dependant processing used typical parameters of sigma=5 (Eq 1. for the continuous calculations) and T=3 for the resim calculations (Eq. 3, both calculations). For the non-continuous calculations, environment residues within 8.0 Angstroms of the CDRs were given proximities of 1.0 whereas environment residues beyond this distance were given proximities of 0.0. FIG. 23 shows that the best human germline acceptors in both of these calculations are the sequence 2-26, h_vh_{—}2-26, because it received the highest resim score in both instances. The second best acceptor selected by the different methods varies depending on which proximity is used. Both calculations use the resim score as a distance-dependent similarity score, but the resim scores may be calculated as a continuous or discrete function of distance. One advantage of the methods of the present invention over methods common in the art is this down-weighing of more distant (less proximal) residues from the patch of interest, which occurs in both the discrete and continuous proximities values.

### EXAMPLE 11

A Preferred Embodiment Compares Framework Regions.

The following hypothetical CDR graft demonstrates why a preferred method of CDR grafting by methods of the present invention, or any other method, compares the similarity of the framework regions instead of the CDR's. This example illustrates why the methods of the present invention are described as comparing the environment residues as opposed to the patch residues in assessing the similarity of two proteins.

For example (FIG. 6), donor antibody's CDRs are to be grafted onto an acceptor, preferably human, germline antibody. Two potential acceptor antibodies are considered. Acceptor (1) has CDRs and framework regions with similarity scores of 90% and 100% to the donor antibody respectively. Acceptor (2) has CDRs and framework regions with similarity scores of 100% and 90% to the donor antibody respectively. These similarity scores may be the scores of the present invention converted into percentages, the percent identities, or any other measure of the similarity of two regions of sequence. Humanization methods that compare framework regions would select acceptor antibody (1) because its framework regions are more similar to the original donor framework regions than are the framework regions from antibody (2). This selection results in a final humanized antibody that is the same as the original donor antibody, having 100% similarity scores to the donor antibody in both the CDRs and framework regions. In contrast, humanization methods that compare CDRs would select acceptor sequence (2). The resulting antibody still has the 100% score to the donor in the CDRs, but has a lower score, 90%, to the donor in the framework region.

The problem with comparing the similarity of CDRs, not framework regions, is that the less preferred protein is used as an example of a stable, antigen-binding, antibody. In comparing CDRs, the method creates an antibody with perfect scores, 100%, to potential acceptor antibody (2). Acceptor (2) is a stable, folded protein, but it is the less preferred reference, or example antibody; it does not contain the strong antigen binding of the original donor antibody. The original donor antibody is recreated by grafting methods that compare framework regions. The original donor antibody is known to be stable and have good antigen-binding properties. Therefore, methods that compare framework regions are less susceptible to losses in antigen binding affinity during humanization. This logic is incorporated into the methods of the present invention and illustrates why, given a patch of residues of interest, the methods of the present invention compare the environment residues surrounding the patch.

### EXAMPLE 12

Heavy Chains

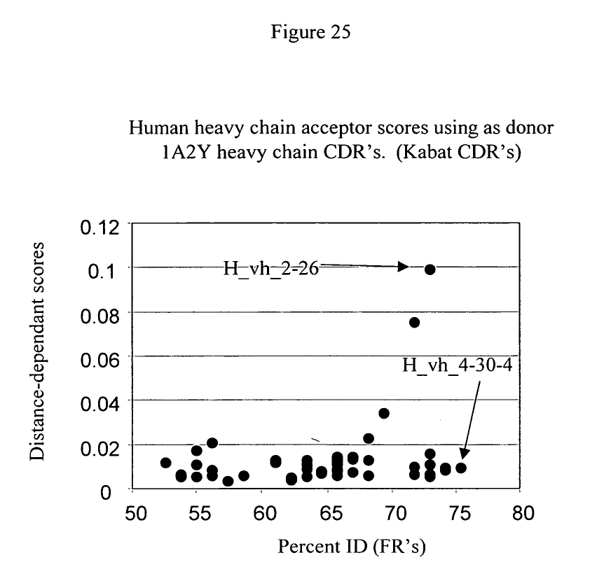

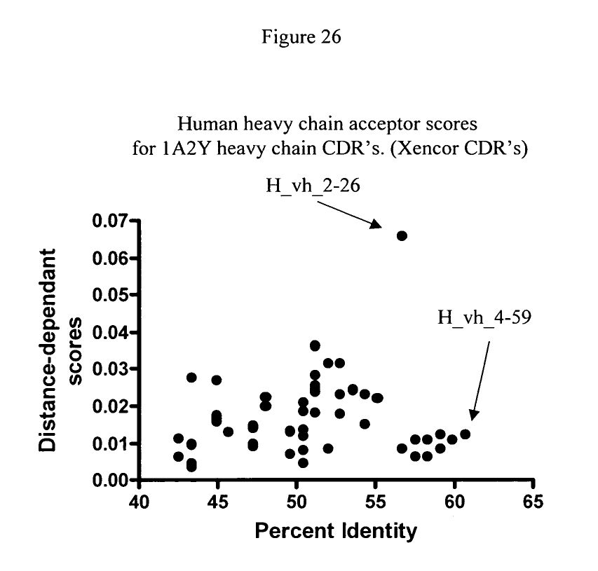

To illustrate the different results obtained with methods of the present invention in comparison to a typical method used in the art, we selected, for example, the best human germline heavy chain sequence to accept the graft of the heavy chain CDRs from an antibody against hen egg white lysozyme (found in crystal structure 1A2Y). 53 human germline heavy chains were considered as potential acceptors of the CDR graft. FIGS. 25 and 26 illustrate their distance-dependant similarity scores from the present invention and compares those scores to their percent identities to the 1A2Y donor. The top-scoring acceptors from the distance-dependant methods and % ID method are labeled. FIG. 25 shows the results of the two methods using Kabat-defined CDRs (Kabat CDRs) and FIG. 26 shows the results of the two methods using Xencor-defined CDRs (Xencor CDRs). Not surprisingly, the distance-dependant scores are not correlated with the percent identities, demonstrating that the two methods use different properties or principles in their analysis. The choice of CDRs also changed the identity of the top-scoring sequences, but the distance-dependant methods of the present invention select a different acceptor than the % ID method using either CDR definition.

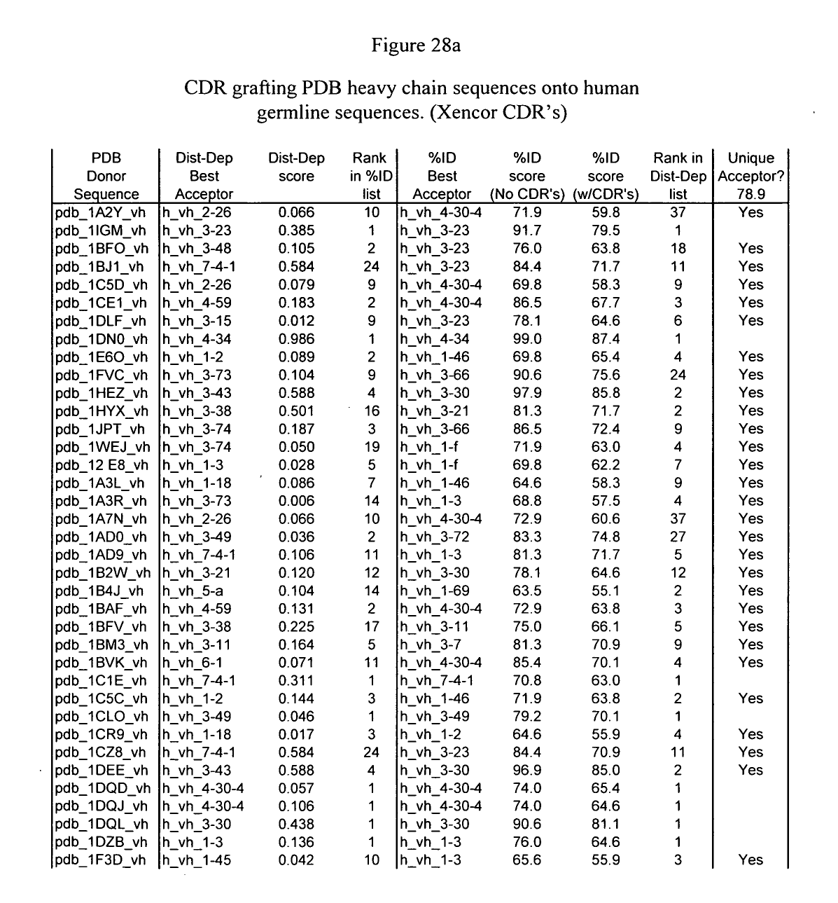

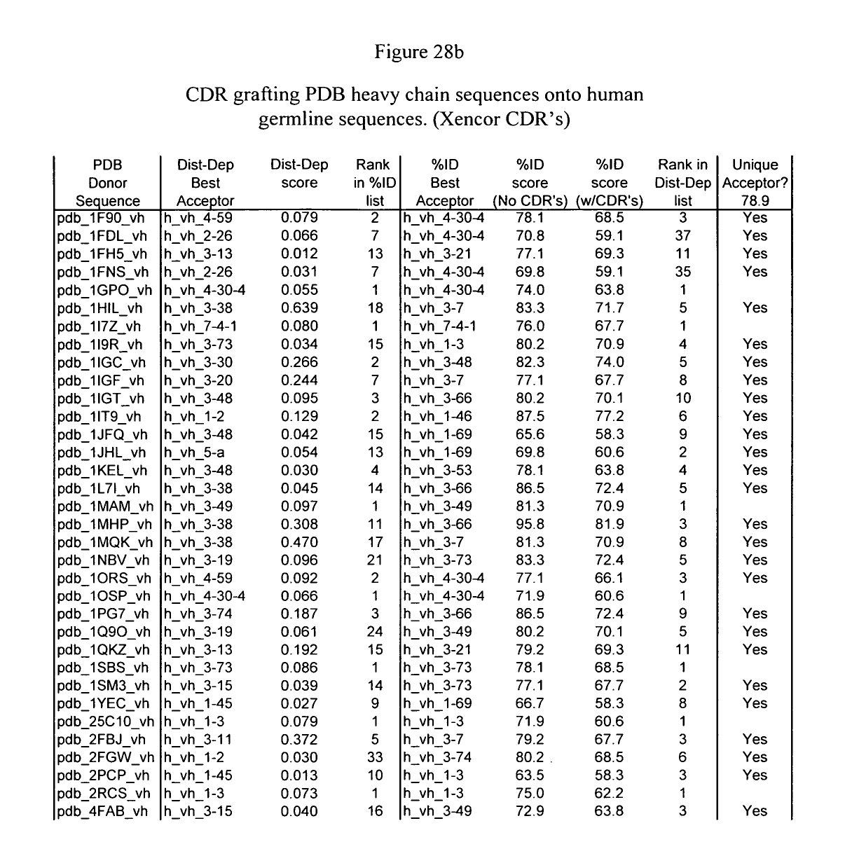

As a further example, the variable heavy chain sequences from 71 antibody structures found in the PDB were used as donor sequences in order to find the best human germline sequence as their acceptor. For each PDB heavy chain donor sequence, the best human acceptor chosen by the methods of the present invention are shown in FIGS. 27 and 28. FIG. 27 shows results obtained using Kabat CDRs and FIG. 28 shows results obtained using Xencor CDRs. For example (FIG. 27), for the first PDB structure listed, 1A2Y, a mouse antibody against hen egg white lysozyme, distance-dependant methods of the present invention predict that the human germline heavy chain sequence, 2-26, is the best acceptor for the CDR graft. Of all of the human germline acceptors, the heavy chain 2-26, “h_vh_{—}2-26”, received the highest resim score, 0.099. If one uses amino acid identity to choose an acceptor, 4-30-4 is chosen as the best acceptor having 65.6% of its amino acids identical to the 1A2Y heavy chain sequence. The two human germline sequences chosen by the two methods differ substantially having 67.7% sequence identity and containing different amino acids at 41 sequence positions. On occasion, the same human germline sequence is chosen with both methods, as is the case with 1IGM as the heavy chain CDR donor. Generally, however, distance-dependant methods (the methods of the present invention) and percent identity select different acceptor sequences. The two methods selected different acceptor sequences for 84.7% of the 71 PDB sequences analyzed with Kabat CDRs and 78.9% of the 71 PDB sequences analyzed with Xencor CDR.

For the best-ranked acceptor as determined by one method, the rank of that sequence determined by the other method is also shown in FIGS. 27 and 28. For example (FIG. 27), h_vh_{—}2-26 (human germline variable heavy chain 2-26) is the best acceptor sequence (ranked #1) for the query sequence 1A2Y as determined by the distance-dependant methods of the present invention. That acceptor, h_vh_{—}2-26, is the 8^{th} best sequence when the % ID method is used to choose the best acceptor. On the other hand, the % ID method chooses h_vh_{—}4-30-4 as the best acceptor, but h_vh_{—}4-59 is the 28^{th} best acceptor as judged by the methods of the present invention. There are 53 human heavy chain germline sequences used in this analysis, so the best sequence as judged by distance-dependant methods ranks in the 8/53=85^{th} percentile of the % ID score. Similarly, the best sequence as judged by the % ID score only ranks in the 28/53=47^{th} percentile of the distance-dependant scores. Amongst the 73 PDB sequences used as CDR donors, the distance-dependant top-scoring acceptor ranks on average only 6.1^{th} in the ranked list from the % ID method, highlighting how the two methods select different sequences.

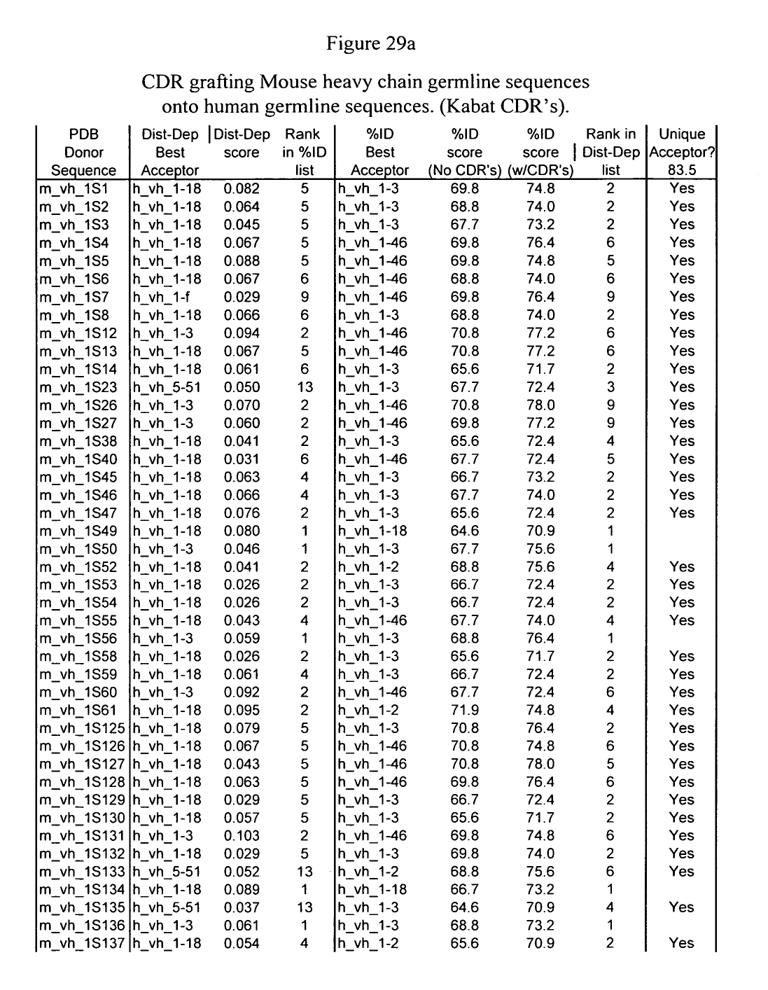

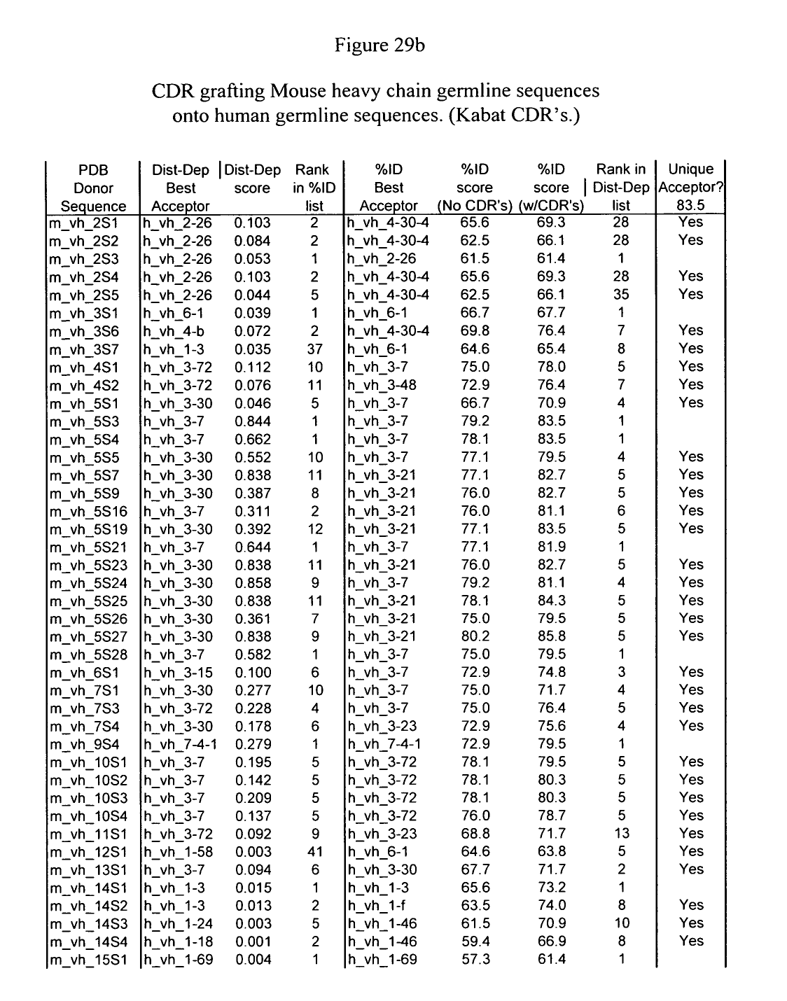

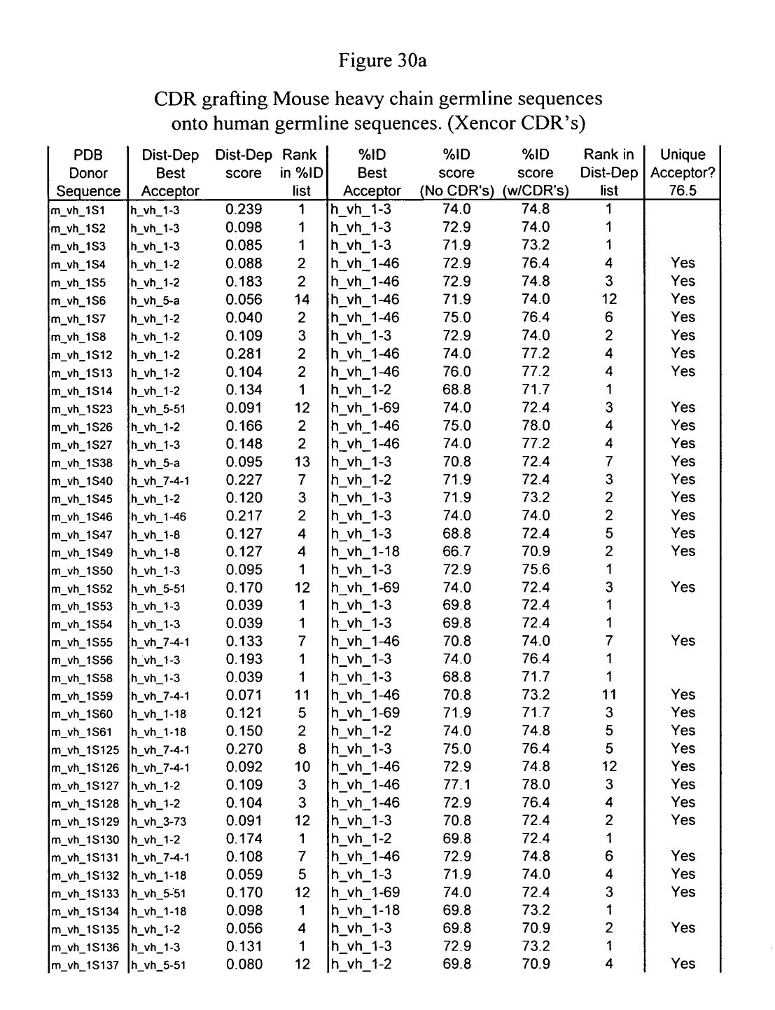

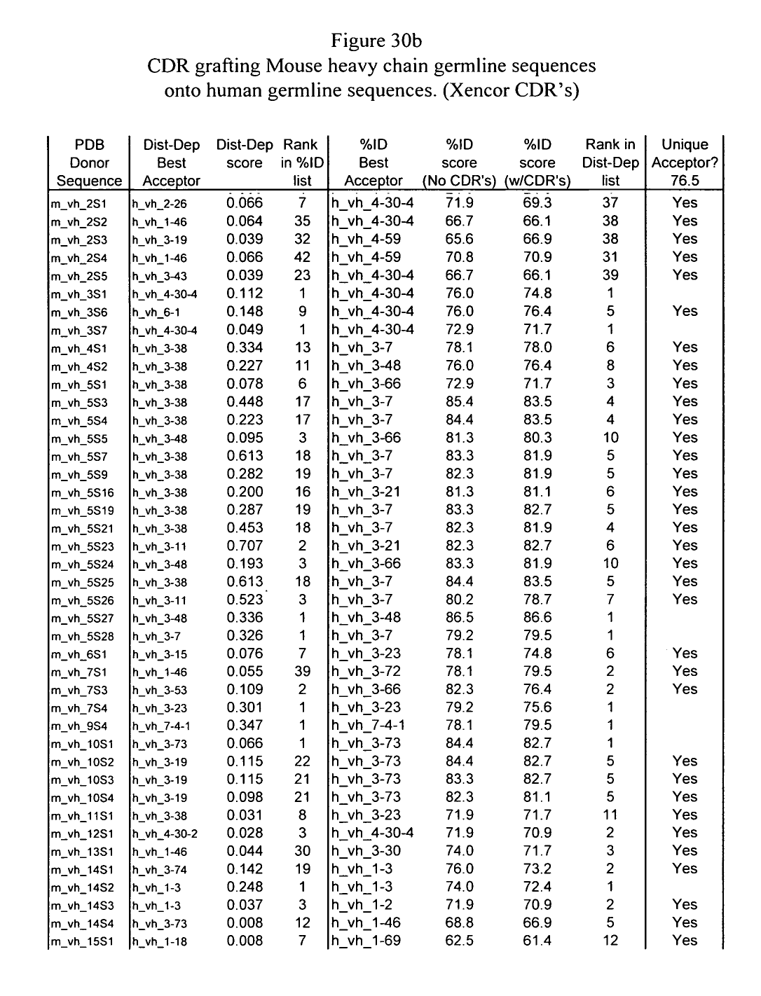

As another example, human germline heavy chain acceptor sequences for CDR grafts were selected from 85 mouse germline heavy chain sequences (FIGS. 29 and 30). Overall, the results are very similar to the PDB queries described above. In 83.5% of the mouse germline query sequences, distance-dependant methods of the present invention and the % ID method suggest different human acceptor sequences. That is, a skilled artisan using the distance-dependant methods of the present invention would use a different acceptor for 83.5% of the CDR grafts than a skilled practioner using the percent identity method. For the mouse germline sequence 1 μl, “m_vh_{—}1S1”, for example (FIG. 29), the distance-dependent methods of the present invention choose 1-18 as the best human germline acceptor, whereas the % ID method chooses 1-3 as the best human germline acceptor. These two human acceptors have different amino acids at 14 positions and 89.0% sequence identity. The best sequence (h_vh_{—}1-18) as judged by the distance-dependant methods for the m_vh_{—}1S1 query is the 5^{th} best sequence as judged by the % ID method. Likewise the best sequence (h_vh_{—}1-3) as judged by the % ID method for the m_vh_{—}1S1 query is the 2^{nd} best sequence as judged by distance-dependant methods. In other examples, say for the query sequence, m_vh_{—}1 S6, the best choice as judged by the distance-dependant methods is not in the top few choices as judged by the % ID method. Amongst the mouse germline query sequences, the distance-dependant top choice ranked on average 5.6^{th} in the % ID list.

Light Chains

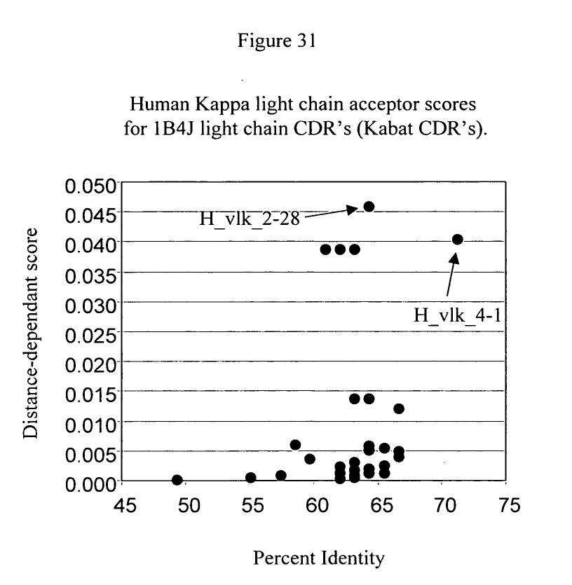

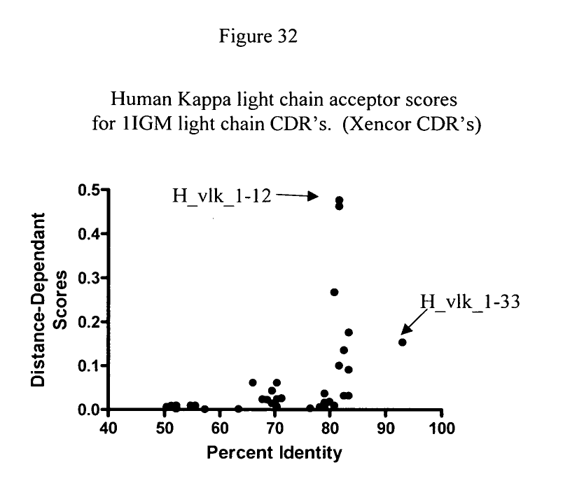

Similar results are obtained when one considers CDR grafts in light chain sequences. For example, FIG. 31 shows the distance-dependant scores of the present invention and % ID scores of 45 potential human kappa light chain acceptors for the donor light chain from PDB structure 1B4J. This analysis used Kabat CDR definitions. FIG. 32 shows the same score using the CDR donor 1IGM and Xencor CDR definitions. With either definition of CDRs, no correlation is found between the distance-dependant and % ID scores. The two methods select different acceptors as their top-ranked choices.

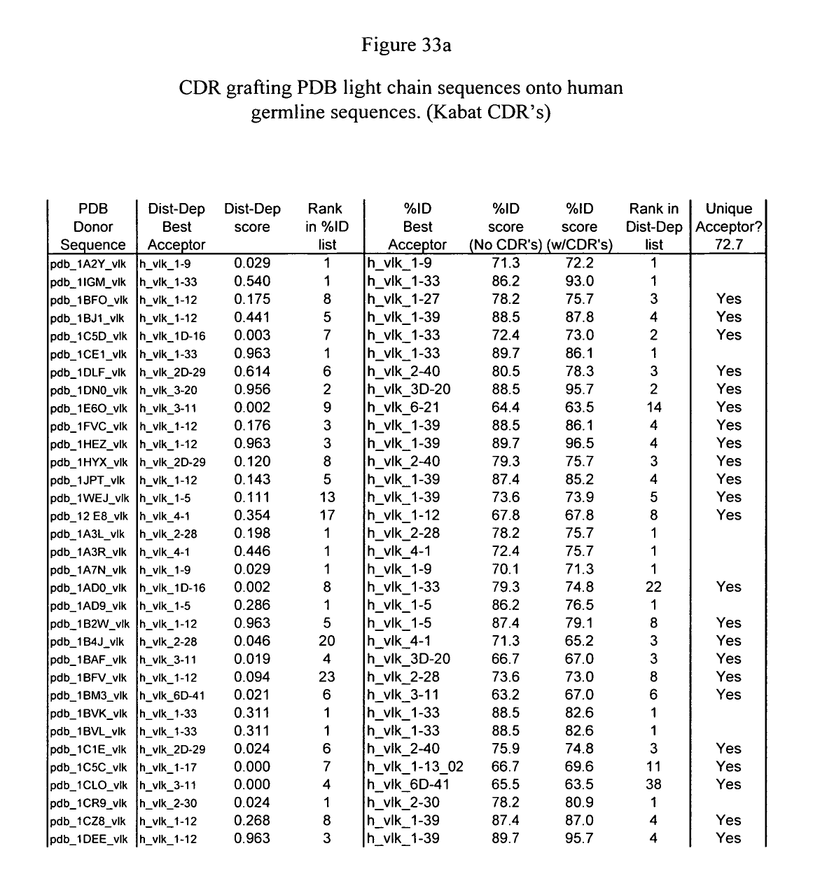

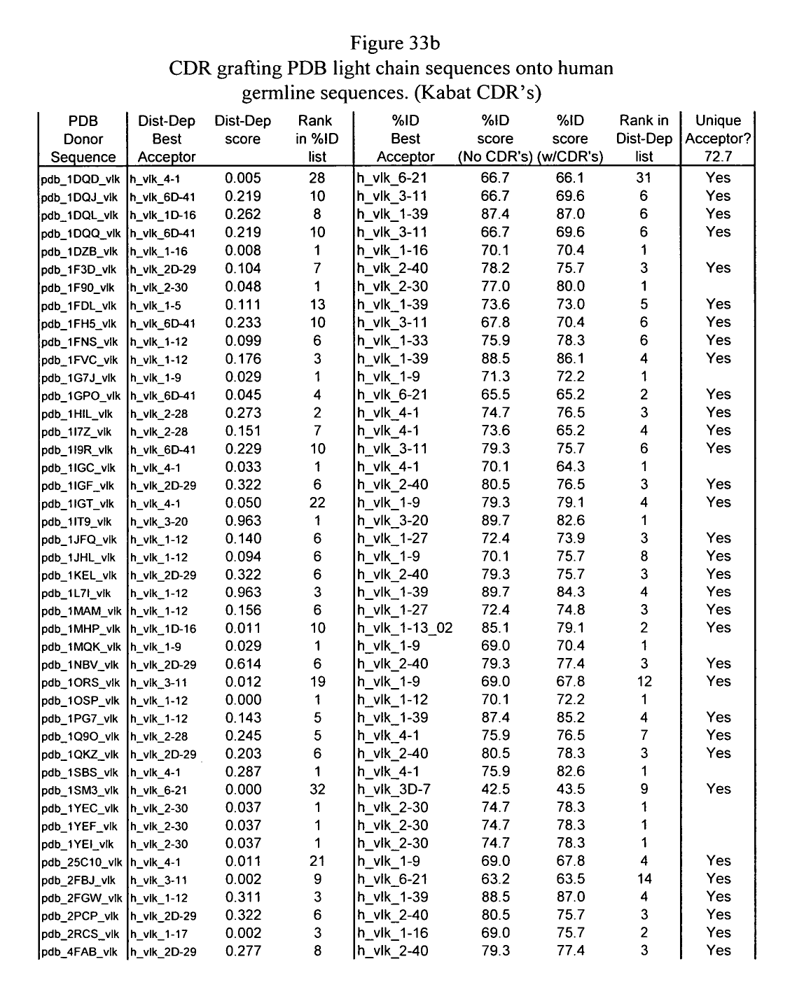

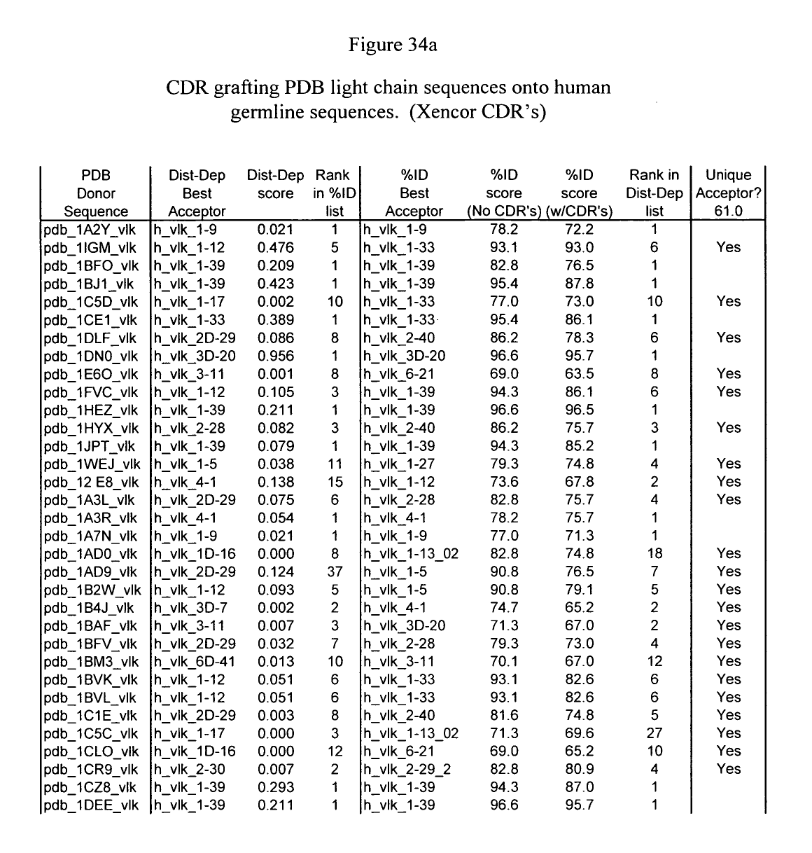

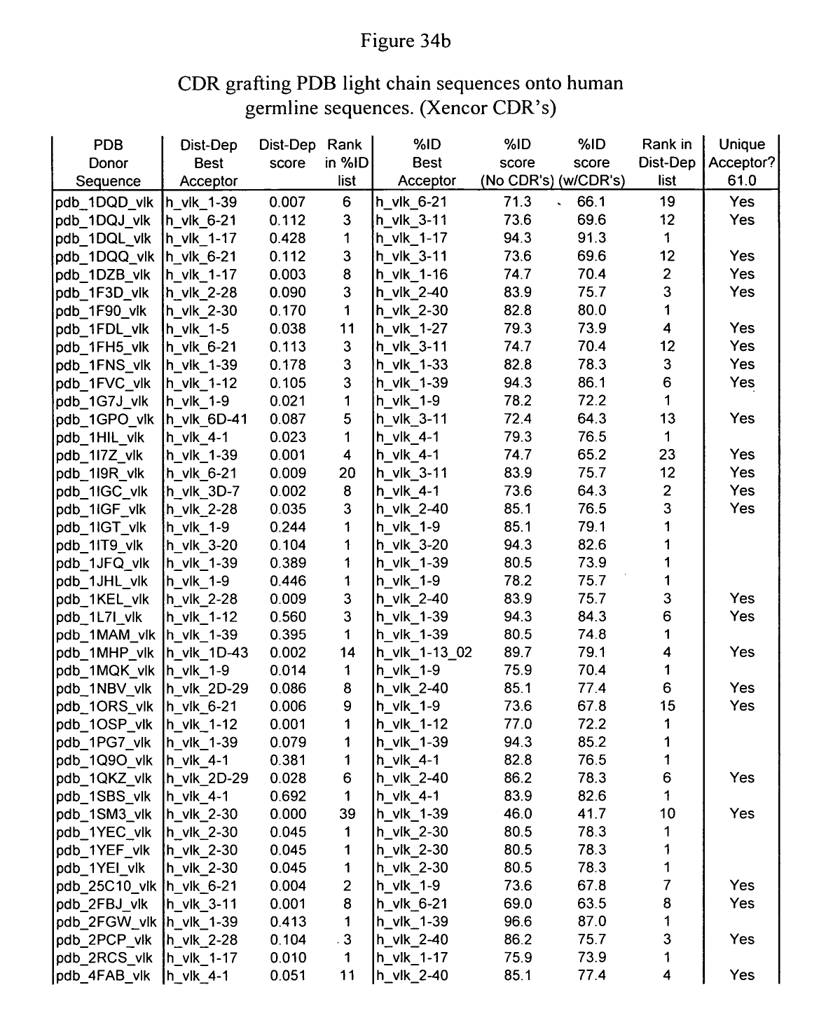

By looking at CDR grafts using the kappa light chain from 77 PDB structures as CDR donors, we found that the two methods choose different acceptors in 72.7% (Kabat CDR definitions) or 61.0% (Xencor CDR definitions) of the grafts. The best acceptors from the two methods for each PDB light chain donor are presented with their scores (FIGS. 33 and 34). On average, the best acceptor, as judged by distance-dependant methods of the present invention, was ranked 6.6th of the 45 human germline kappa light chains as judged by the % ID method (Kabat CDRs). Likewise, on average, the best acceptor, as judged by the % ID method, was ranked 4.8th, as judged by the distance-dependant methods (kabat CDRs). Using Xencor CDRs, these two average ranks are similar, 5.2^{th} and 5.0^{th}.

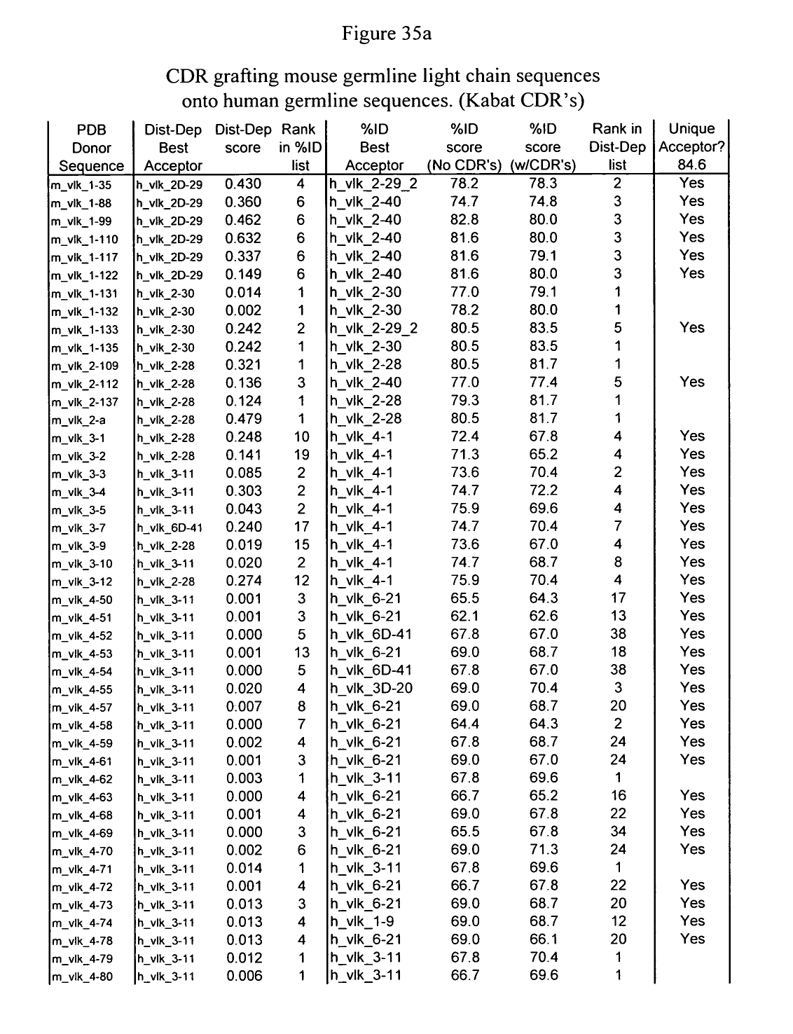

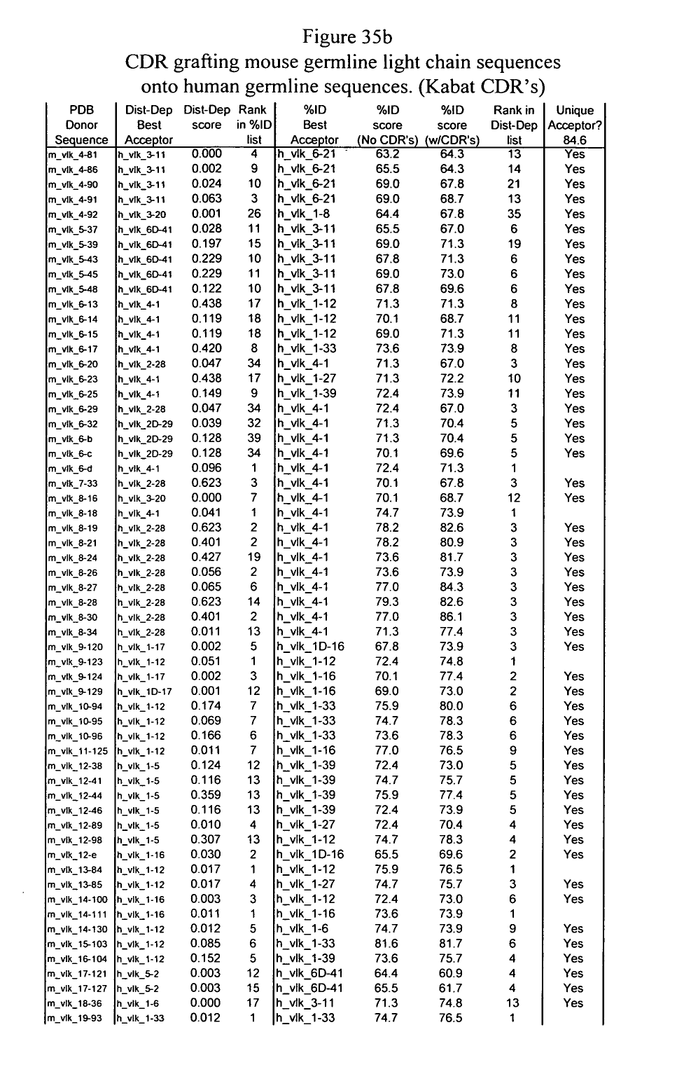

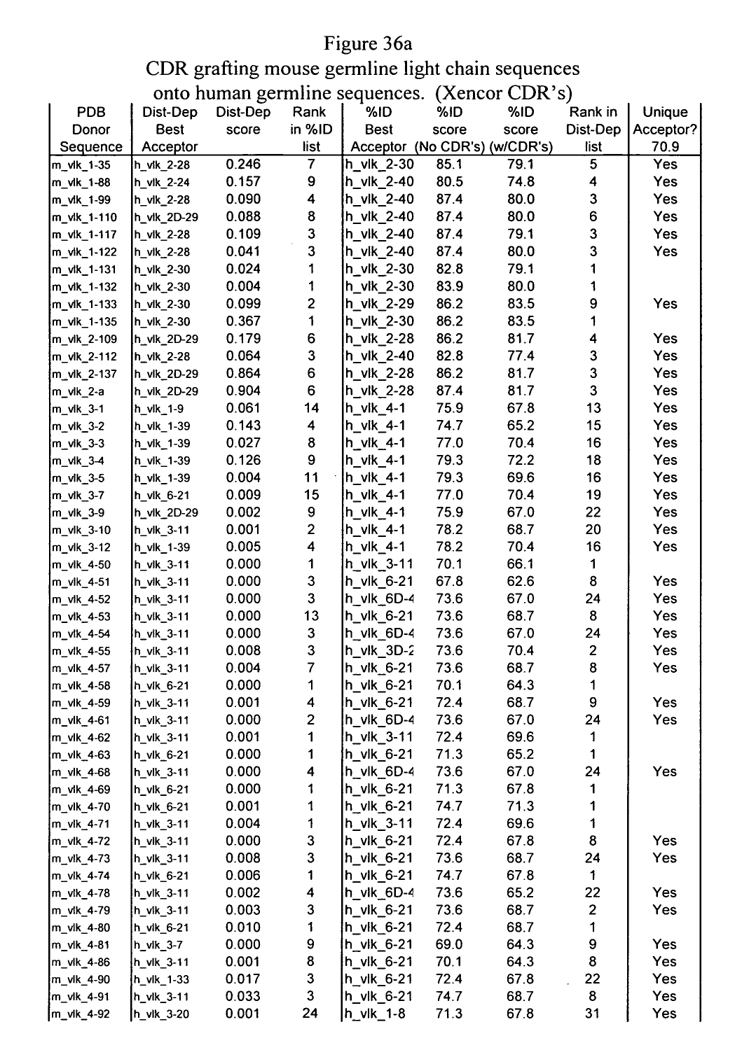

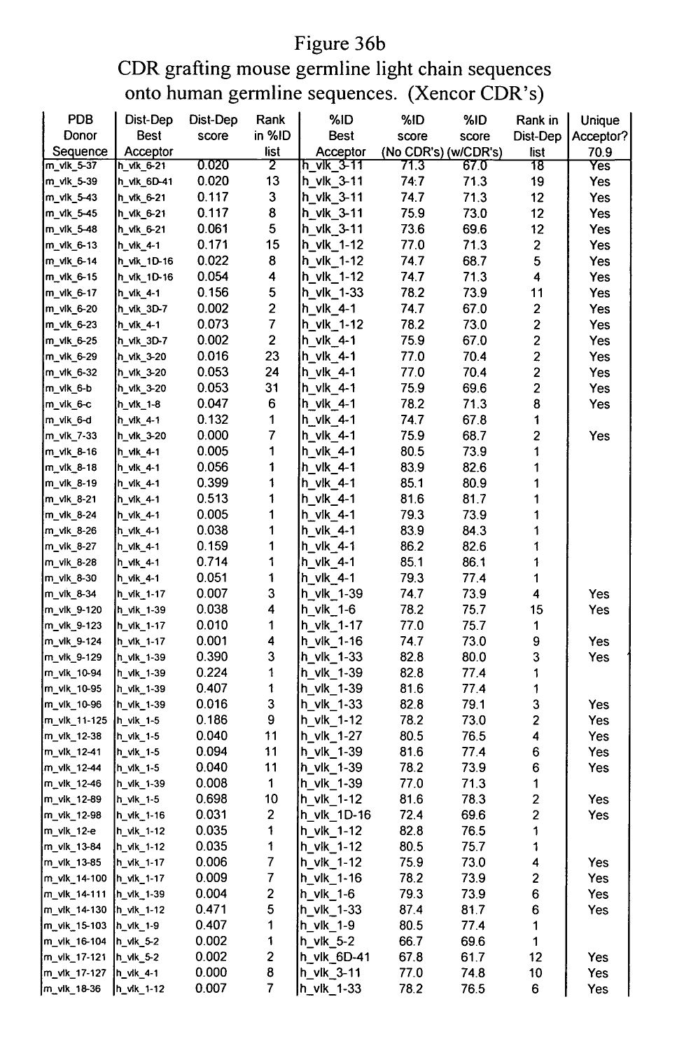

CDR grafts of light chain CDRs from mouse germline donors show similar results to the PDB light chain donors (FIGS. 35 and 36). Using Kabat CDRs, the distance-dependant methods of the present invention and % ID methods choose a different human light chain germline acceptor for 84.6% of the 103 mouse germ line donors. On average, the best acceptor, as judged by distance-dependant methods, was ranked 8.1^{st} of the 45 human germline kappa light chains, as judged by the % ID method. Likewise, on average, the best acceptor, as judged by the % ID method, was ranked 7.9th, as judged by the distance-dependant methods. Using Xencor CDRs, the two methods choose a different human light chain germline acceptor for 70.9% of the 103 mouse germline donors and the two average ranks are 5.2th and 6.9^{th}, further illustrating how the distance-dependant methods of the present invention select new sequences to be used as CDR acceptors.

Whereas particular embodiments of the invention have been described above for purposes of illustration, it will be appreciated by those skilled in the art that numerous variations of the details may be made without departing from the invention as described in the appended claims. All references cited herein, including patents, patent applications (provisional, utility and PCT), and publications are incorporated by reference in their entirety.

We claim:

1. A method of designing a humanized antibody variable domain for a target antigen, said method comprising:
a) providing structural data comprising a reference set of the measure of the distances between at least one amino acid residue and other amino acid residues in a reference antibody variable domain, said domain comprising complementary determining regions (CDRs) and framework regions (FRs);
b) providing the amino acid sequence of a donor, non-human antibody variable domain comprising donor CDRs and donor FRs;
c) providing a plurality of amino acid sequences of acceptor human antibody variable domains comprising acceptor CDRs and acceptor FRs;
d) calculating suitability scores from said plurality using distance-weighted similarity scores and identifying a best acceptor domain using said suitability scores;
e) replacing said acceptor human antibody CDRs of said best acceptor domain with said donor CDRs to form a humanized antibody variable domain amino acid sequence.

2. A method according to claim 1 further comprising inputting said structural data into a computer and computationally calculating a best acceptor domain.

3. A method according to claim 2 wherein said structural data comprises the three-dimensional coordinates of said reference variable domain.

4. A method according to claim 1 wherein said reference set comprises a measure of the distances of every residue with every other residue of the reference domain.

5. A method according to claim 1 wherein said structural data comprises a distance-matrix of said variable domain.

6. A method according to claim 2 wherein said reference domain and said donor domain are the same, and steps a) and b) are done simultaneously by inputting the three-dimensional coordinates of said donor domain.

7. A method according to claim 2 wherein said reference domain and one of said acceptor domains are the same, and steps a) and c) are done simultaneously by inputting the three-dimensional coordinates of said acceptor domain.

8. A method according to claim 1 further comprising synthesizing said humanized variable domain.

9. The method of claim 1, wherein said reference domain, said donor domain and said acceptor domains comprise heavy chain variable domains.

10. The method of claim 8 wherein said CDRs comprise residues 27-35, 52-56 and 95-102 using the number of Kabat et al.

11. The method of claim 1, wherein said reference domain, said donor domain and said acceptor domains comprise light chain variable domains.

12. The method of claim 11 wherein said CDRs comprise residues 27-32, 50-56 and 91-97 using the number of Kabat et al.

13. The method of claim 1, wherein said reference domain is a consensus variable domain.

14. The method of claim 1, wherein said donor domain is a mouse variable domain.

15. The method of claim 1 wherein said acceptor variable domains are human germline variable domains.

16. The method of claim 1, wherein said distance-weighted similarity scores are calculated such that residues within about 10 Angstroms of the residues of the CDRs are given full weight and residues not within about 10 angstroms are given zero weight.

17. The method of claim 1, wherein said distance-weighted similarity score utilizes weights calculated as a non-discrete function of distance.

18. The method of claim 1, wherein said distance-weighted similarity scores are calculated wherein the weights of residues are inversely proportional to the distance of said residues from the residues of the CDRs.

19. The method of claim 18, wherein said weights of residues decreases exponentially with the distances of residues to the CDRs.

20. The method of claim 18, wherein said weights of residues decreases linearly with the distances of residues to the CDRs.

21. The method of claim 18, wherein said distance-weighted similarity scores are resim scores.
